(19) United States
(12) Patent Application Publication
(10) Pub. No.: US 2008/0063600 A1
(43) Pub. Date: Mar. 13, 2008

(54) Diagnostics And Therapeutics For Transmissible Spongiform Encephalopathy And Methods For The Manufacture Of Non-Infective Blood Products And Tissued Derived Products

(75) Inventors: Adriano P. Aguzzi, Zurich (CH); Michael A. Klein, Rheinfelden (DE); Alex J. Raeber, Bottighofen (CH); Charles Weissmann, London (GB); Rolf M. Zinkernagel, Zumikon (CH)

Correspondence Address: DYKEMA GOSSETT PLLC, 10 S. WACKER DR., STE. 2300, CHICAGO, IL, 60606, US

(73) Assignee: ABBOTT LABORATORIES, Abbott Park, IL (US)

(21) Appl. No.: 11/855,885

(22) Filed: Sep. 14, 2007

Related U.S. Application Data
(63) Continuation of application No. 09/554,567, filed on Sep. 1, 2000, now abandoned.

(30) Foreign Application Priority Data
Dec. 16, 1997 (EP) 97122186.6

(51) Int. Cl. A61K 31/56; A61K 38/00; A61K 39/395; A61K 49/00; A61P 43/00; C07K 16/00; C12N 5/06; G01N 33/53

(52) U.S. Cl. 424/9.1; 424/130.1; 424/141.1; 435/372.2; 435/372.3; 435/7.1; 514/177; 514/9; 514/90; 530/387.1

(57) ABSTRACT

B-cells have been identified as being the crucial carriers of infectivity in the spread of transmissible spongiform encephalopathy within an infected organism. In a second step, B-cells may infect further components_of the immune system, e.g. T-cells. Accordingly, the present invention provides B-cell and T-cell specific ligands for the use in diagnostics and therapeutics for transmissible spongiform encephalopathy and provides methods for the manufacture of non-infective blood products and tissue derived products. Thus, the present invention provides medicaments comprising B-cell and/or T-cell depletants, for the treatment of pathologies where the depletion of B-cells and/or T-cells, and more particularly of tse-infected B-cells and/or T-cells is therapeutically effective.

### RELATED APPLICATIONS

This application is a continuation of U.S. Ser. No. 09/554,567, filed May 16, 2000, which claims priority to Patent Cooperation Treaty Application No. PCT/EP98/08271, filed Dec. 16, 1998, now expired, and European Patent Application No. EP97122186.6, filed Dec. 16, 1997, currently pending, of which all are incorporated herein by reference in their entireties.

### FIELD OF THE INVENTION

The present invention relates to diagnostics of and therapeutics for transmissible spongiform encephalopathy (tse). Further, the invention relates to non-infective body fluid products and to non-infective tissue derived products and to-suitable methods for the manufacture thereof.

### BACKGROUND ART

Transmissible spongiform encephalopathies (TSE's) comprise a group of slow degenerative diseases of the CNS such as Creutzfeldt-Jakob disease (CJD), new variant CJD (termed nvCJD)^{1,2}, Gerstmann-Sträussler-Scheinker disease (GSS) and kuru in man and scrapie in sheep or BSE (mad cow disease) in cattle.

The occurrence of these exotic illnesses is still fortunately very low, probably occurring at 1:1,000,000 but there are striking similarities when compared to Alzheimer's disease. However, BSE reached epidemic proportions in England and was spread by the use of rendered materials in cattle-feed. Dairy cattle in particular are at the highest measurable risk. A tragically similar incidence has occurred with humans.

During the production of human growth hormone from human glands collected from cadavers, the pathogenic agent of Creutzfeldt-Jakob disease was introduced. Several cases have now been reported in patients treated with this growth hormone. The patients were predominantly children, whereas the disease normally attacks adults over 50 years of age. From a general point of view, it appears that peripheral, in particular oral uptake of tse-infected material is epidemiologically most relevant, as at least in the case of BSE/sheep scrapie, kuru and likely nvCJD.

These examples point out the potential danger of these new diseases and the difficulties in diagnosing and treating them effectively.

The unusual properties of the pathogenic agent, designated as “prion” (Prusiner, S. B. Novel proteinaceous infectious particles cause scrapie. Science 216, 136-144. (1982)) include the extremely long incubation periods, exceeding one year, and resistance to high temperatures, formaldehyde treatment and UV irradiation (Gordon, W. S. Vet Rec 58,516 (1946); Pattison, I. H. Resistance of the scrapie agent to formalin. J Comp Pathol 75, 159-164 (1965); Alper et al., The exceptionally small size of the scrapie agent. Biochem. Biophys. Res. Commun. 22, 278-284 (1966); Latarjet et al., Inactivation of the scrapie agent by near monochromatic ultraviolet light. Nature 227, 1341-1343 (1970)). Speculations arose early on that the scrapie agent might be devoid of nucleic acid (Alper et al., Does the agent of scrapie replicate without nucleic acid? Nature 214, 764-766 (1967); Gibbons, R. A. and Hunter, G. D. Nature of the scrapie agent. Nature 215, 1041-1043 (1967); Pattison, I. H. and Jones, K. M. The possible nature of the transmissible agent of scrapie. Vet. Rec. 80, 2-9 (1967)). Considerable evidence now supports the “protein only” hypothesis (Prusiner, S. B. and Hsiao, K. K. Human prion diseases. Ann. Neurol. 35, 385-395 (1994); Weissmann, C. Molecular biology of prion diseases. Trends Cell Biol. 4, 10-14 (1994)) which proposes that the prion is devoid of nucleic acid and identical with PrP^{Sc}, a modified form of PrP^{c}. PrP^{c} is a normal host protein (Oesch et al., A cellular gene encodes scrapie PrP 27-30 Protein. Cell 40, 735-746 (1985); Chesebro et al., Identification of scrapie prion protein-specific mRNA in scrapie-infected and uninfected brain. Nature 315, 331-333 (1985)) found predominantly on the outer surface of neurons, but also in many other tissues (Manson et al., The prion protein gene: a role in mouse embryogenesis? Development 115, 117-122 (1992); Bendheim et al., Nearly ubiquitous tissue distribution of the scrapie agent precursor protein. Neurology 42, 149-156 (1992)). PrP^{Sc} is defined as a protease-resistant form of PrP^{c} which readily forms aggregates after detergent treatment (Mc Kinley et al., Scrapie prion rod formation in vitro requires both detergent extraction and limited proteolysis: J. Vitrol. 65, 1340-1351 (1991)). No chemical differences have so far been detected between PrP^{Sc} and PrP^{c} (Stahl et al., Structural studies ‘of the scrapie prion, protein using mass spectrometry and amino acid sequencing. Biochemistry 32, 1991-2002 (1993)). Prusiner proposed that PrP^{Sc}, when introduced into a normal cell, causes the conversion of PrP^{c} or its precursor into PrP^{Sc} (Oesch et al., Search for a scrapie-specific nucleic acid: a progress report. Ciba. Found. Symp. 135, 209-223 (1988); Prusiner et al., Transgenetic studies implicate interactions between homologous PrP isoforms in scrapie prion replication. Cell 63, 673-686 (1990); Bolton, D. C. and Bendheim, P. E. A modified host protein model of scrapie. Ciba. Found. Symp. 135, 164-181 (1988)). The conversion is believed to result from a conformational rearrangement of PrP^{c}. Some researchers still adhere to the virino hypothesis which holds that the infectious agent consists of a nucleic acid genome and the host-derived PrP, which is recruited as some sort of coat (Dickinson, A. G. and Outram, G. W. Genetic aspects of unconventional virus infections: the basis of the virino hypothesis. Ciba. Found. Symp. 135, 63-83 (1988); Hope, J. The nature of the scrapie agent: the evolution of the virino. Ann. N.Y. Acad. Sci. 724, 282-289 (1994)). Finally, the possibility that the infectious agent is a virus with unusual properties is still upheld by some (Diringer et al., The nature of the scrapie agent: the virus theory. Ann. N.Y. Acad. Sci. 724, 246-258 (1994); Pocchiari, M. Prions and related neurological diseases. Molec. Aspects. Med. 15, 195-291 (1994); Rohwer, R. G. The scrapie agent: “a virus by any other name”. Curr. Top. Microbiol. Immunol. 172, 195-232 (1991)). No credible evidence for the existence of a scrapie-specific nucleic acid, as demanded by the virus and the virino hypotheses, has yet been forthcoming (Oesch et al., vide supra; Kellings et al., Further analysis of nucleic acids in purified scrapie prion preparations by improved return refocusing gel electrophoresis. J. Gen. Virol. 73, 1025-1029 (1992)).

Prusiner and his colleagues were the first to purify PrP^{Sc} and demonstrate physical linkage to scrapie infectivity (Bolton et al., Identification of a protein that purifies with the scrapie prion. Science 218, 1309-1311 (1982)). A collaboration between the groups of Prusiner, Hood and Weissmann led to the isolation of PrP cDNA and to the realization that PrP^{c} was a normal host protein and that PrP^{Sc} was an isoform of PrP^{c} (Oesch et al., vide supra (1985)). Weissmann and his collaborators (Basler et al., Scrapie and cellular PrP isoforms are encoded by the same chromosomal gene. Cell 46, 417-428 (1986)) cloned the PrP gene (PrnP) and Prusiner's group showed the linkage between genetic susceptibility to prion disease and the PrnP gene in mouse (Prusiner et al., vide supra (1990)) and man (Hsiao et al., Linkage of a prion protein missense variant to Gerstmann-Straussler syndrome. Nature 338, 342-345 (1989)). Several groups reported physical data supporting conformational differences between PrP^{c} and PrP^{Sc} (Caughey et al., Secondary structure analysis of the scrapie-associated protein PrP 27-30 in water by infrared spectroscopy. Biochemistry 30, 7672-7680 (1991); Cohen et al., Structural clues to prion replication. Science 264, 530-531 (1994); Huang et al., Proposed three-dimensional structure for the cellular prion protein. Proc. Natl. Acad. Sci. U.S.A. 91, 7139-7143 (1994); Pan et al., Conversion of alpha-helices into beta-sheets features in the formation of the scrapie prion proteins. Proc. Natl. Acad. Sci. U.S.A. (1993); Safar et al., Conformational transitions, dissociation, and unfolding of scrapie amyloid (prion) protein. J. Biol. Chem. 268, 20276-20284 (1993)).

Since there is no reliable marker of transmissible spongiform encephalopathy infectivity, the kinetics of replication of the infectious agent cannot be studied specifically since the physical carriers of prions are not known. However, an increasing body of evidence from early experiments and from receipt studies points to the importance of two distinct phases of replication during the life cycle of the prion, the infectious agent causing’ spongiform encephalopathies. In the first phase, replication of infectivity is thought to take place primarily in lymphoid organs (Eklund et al., Pathogenesis of scrapie virus_infection in the mouse. J. Infect. Dis. 117, 15-22 (19673; Clarke, M. C. and Kimberlin, R. H. Pathogenesis of mouse scrapie: distribution of agent in the pulp and stroma of infected spleens. Vet. Microbiol. 9, 215-225 (1984); Fraser, H. and Dickinson, A. G. Studies of the lymphoreticular system in the pathogenesis of scrapie’: the role of spleen and thymus. J. Comp. Pathol. 88, 563-573 (1978)). For example, infectivity can be demonstrated in the spleen as early as 4 days after i.p. or i.e. infection, after which a plateau is quickly reached. This is true even if infection takes place via the intracerebral route (Kimberlin, R. H. and Walker, C. A. Pathogenesis of experimental scrapie. Ciba. Found. Symp. 135, 37-62 (1988)), and replication of the infectious agent in the spleen precedes intracerebral replication even if infectivity is administered intracerebrally (Rubenstein et al., Scrapie-infected spleen: analysis of infectivity, scrapie-associated fibrils, and protease-resistant proteins. J. Infect. Dis. 164, 29-35 (1991)). Infectivity can also accumulate in other components of the lymphoreticular system (LRS), e.g. in lymph nodes and in Peyer's plaques of the small intestine, where replication of infectivity can be demonstrated almost immediately following oral administration of prion preparations. The extremely rapid establishment of a plateau of the infectious titer in the spleen at a relatively early time point during the latency time suggests that the availability of prion replication sites is rate-limiting in the LRS. It is not known however, whether this plateau is due to a limited number of spleen cells supporting prion replication, or rather to limited availability of prion replication sites within each cell.

The nature of the cells supporting prion replication within the LRS is uncertain. Indirect evidence obtained by studies in which the spleen was removed at variable intervals after i.p. infection suggests that the critical tissue compartment is long-lived and does not consist primarily of lymphocytes. In addition, ablation of lymphocytes by total body irradiation does not seem to affect the incubation time of mouse scrapie thereby disproving involvement of this cell type. (Fraser et al., The scrapie disease process is unaffected by ionising radiation. Prog. Clin. Biol. Res. 317, 653-658 (1989)). Taken together, these and other findings suggest that follicular dendritic cells (FDC) may be the main population of cells involved in LRS replication of prions. Indeed, PrP accumulates in FDCs in the spleen of wild-type and nude mice, and i.p. infection does not lead to cerebral scrapie in SCID mice (whose FDCs are thought to be functionally impaired) while it efficiently provokes the disease in nude mice which bear a selective T-cell defect (Muramoto et al., Species barrier prevents an abnormal isoform of prion protein from accumulating in follicular dendritic cells of mice with Creutzfeldt-Jakob disease. J. Virol. 67, 6808-6810 (1993)).

Though above delineated steps are thought to be important in the natural history of transmissible spongiform encephalopathy within an infected organism, the limiting factor or physical entity involved in the development and spread of transmissible spongiform. Encephalopathy_after peripheral infection, that is to say the physical carrier of the prion, is still not known. Even though precise monitoring of the epidemic spread of transmissible spongiform encephalopathy is rendered extremely difficult by the long incubation times involved (up to 30 years), it appears to be likely that peripheral infection, e.g. by alimentary exposure, is the most relevant route of propagation. Any attempt to combat transmissible spongiform encephalopathy should thus focus on such limiting factors or physical entities involved in the development of the disease after peripheral infection. However, detailed knowledge about such limiting factor(s) or entities is an essential prerequisite to the design of improved therapeutic approaches aimed at interfering with prion replication and spread within an infected victim.

Though a first therapeutic approach based on the administration of prednisolone as immunosuppressant has been recently proposed by Aguzzi et al. in Lancet 350:1519-1520 (1997), the treatment proposed is relatively crude and should be regarded as provisional since it affects many cell types in addition to the unknown limiting factors and physical entities likely to be directly involved in prion spread and replication. Therefore, there is an urgent need to precisely target the rate limiting steps in prion spread, since only exact knowledge of the main bottle neck involved, would allow its more or less selective closing by therapeutical means.

Further, knowledge about the identity of the physical carriers of prions would allow the design of improved assay methods for determining the infectivity of potentially infective, materials like blood products or tissue derived products and for an improved monitoring of the epidemic progress of transmissible spongiform encephalopathy within infected populations. Also, knowledge about the interaction of the physical carriers of prions with further physical entities involved in pathogenesis would allow the monitoring of the disease progress within an infected victim and/or the verification of the effectiveness of therapeutic treatment.

Still further, once the identity of the physical carriers is known, suitable methods for the separation of said physical carriers of prions from body fluid or tissue derived products intended for medical use or industrial application may be tailored on demand.

Accordingly, there is an urgent need for the specific identification of the limiting factors and physical entities in the development of spongiform encephalopathy after peripheral infection. There is further a need for providing improved medicaments for combating spongiform encephalopathy in infected organisms, that is to say humans and animals. Still further, there is a need for providing improved assay methods for the diagnosis and/or monitoring of the progress or regress of transmissible spongiform encephalopathy in infected organisms or in organisms suspected of being infected. Such assay methods are also needed for the safety testing of body fluid or tissue derived products derived from such organisms. Still further, there is a need for providing body fluid or tissue derived products which are not tse-infective in order to prevent the further spread of transmissible spongiform encephalopathy within the infected human and animal populations. Still further, there is a need for providing a method for the manufacture of such uninfective body fluid or tissue derived products. Still further there is a need for proving suitable reagents (i.e. ligands, like e.g. antibodies) being capable of recognizing the crucial physical entity involved in the spread of spongiform encephalopathy.

Satisfaction of above needs as well as of further needs which will become apparent hereinafter is an object of the present invention.

### SUMMARY OF THE INVENTION

In order to meet above objects and to satisfy above needs, in one embodiment, the present invention provides a medicament comprising B-cell depletants for the treatment of pathologies where the depletion of B-cells, and more particularly of infective B-cells is therapeutically effective.

In a further embodiment, the present invention provides the use of B-cell depletants for the manufacture of a medicament for the treatment or prevention of transmissible spongiform encephalopathy in infected humans or animals. Preferred B-cell depletants are anti B-cell antibodies or B-cell depleting drugs, comprising e.g. chemical compounds.

In a further embodiment, the present invention provides a medicament comprising T-cell depletants for the treatment of pathologies where the depletion of T-cells, and more particularly of infective T-cells is therapeutically effective.

In a further embodiment, the present invention provides the use of T-cell depletants for the manufacture of a medicament for the treatment or prevention of transmissible spongiform encephalopathy in infected humans or animals. Preferred T-cell depletants are anti T-cell antibodies or T-cell depleting drugs, comprising e.g. chemical compounds.

In a further embodiment, the present invention provides a product comprising cyclophosphamide and dexamethasone as a combined preparation for the simultaneous, separate or sequential use in the treatment or prevention, of transmissible spongiform encephalopathy in infected humans or animals.

In a further embodiment, the present invention provides the use of a combination of cyclophosphamide and dexamethasone either in a combined dosage form or in separate dosage forms for the manufacture of a medicament for the treatment or prevention of transmissible spongiform encephalopathy in infected humans or animals.

In a further embodiment, the present invention provides an assay method for determination of the presence of tse-infected B-cells in humans or animals or in body fluid or tissue derived products isolated therefrom. Preferred assay methods comprise infectivity bioassays or Western blots carried out with presumably tse-infected B-cells.

In a further embodiment, the present invention provides an assay method for determination of the presence of tse-infected T-cells in humans or animals or in body fluid or tissue derived products isolated therefrom. Preferred assay methods comprise infectivity bioassays or Western blots carried out with presumably tse-infected T-cells.

In a further embodiment, the present invention provides an assay method for the monitoring of the progress of transmissible spongiform encephalopathy in humans or animals.

In a further embodiment, the present invention provides an assay method for the monitoring of tse therapy.

In a further embodiment, the present invention provides a body fluid or tissue derived product, characterized in that it has been depleted from B-cells in vitro. A preferred B-cell depleted product is B-cell depleted buffy coat.

In a further embodiment, the present invention provides the use of B-cell depleted body fluid or tissue derived products for the prevention of transmissible encephalopathy spread in human or animal populations. Preferably, such B-cell depleted products are products containing cells or cell debris.

In a further embodiment, the present invention provides a body fluid or tissue derived product, characterized in that it has been depleted from T-cells in vitro. A preferred T-cell depleted product is T-cell depleted buffy coat.

In a further embodiment, the present invention provides the use of T-cell depleted body fluid or tissue derived products for the prevention of transmissible encephalopathy spread in human or animal populations. Preferably, such T-cell depleted products are products containing cells or cell debris.

In a further embodiment, the present invention provides a method for the manufacture of a body fluid or tissue derived product, characterized in that said method comprises a step of separating B-cells from said body fluid or tissue derived product. Preferred methods concern the separation of B-cells from plasma and from buffy coat.

In a further embodiment, the present invention provides a method for the manufacture of a body fluid or tissue derived product, characterized in that said method comprises a step of separating T-cells from said body fluid or tissue derived product. Preferred methods concern the separation of T-cells from plasma and from buffy coat.

In a further embodiment, the present invention provides a method for the manufacture of a body fluid or tissue derived product, characterized in that said body fluid or tissue derived product is isolated from B-cell-deficient humans or animals. Preferred body fluid derived products are plasma or buffy coat.

In a further embodiment, the present invention provides an antibody directed against tse-infected B-cells.

In a further embodiment, the present invention provides the use of an antibody directed against tse-infected B-cells in a diagnostic assay.

In a further embodiment, the present invention provides a medicament comprising an antibody directed against tse-infected B-cells.

In a further embodiment, the present invention provides an antibody directed against tse-infected T-cells.

In a further embodiment, the present invention provides the use of an antibody directed against tse-infected T-cells in a diagnostic assay.

In a further embodiment, the present invention provides a medicament comprising an antibody directed against tse-infected T-cells.

In a further embodiment, the present invention provides a ligand capable of identification of tse-infected B-cells, characterized in that specific interaction between said ligand and said tse-infected B-cell is based on the infectivity of said B-cell.

In a further embodiment, the present invention provides the use of a ligand as above in a method of analysis of said tse-infected B-cell.

In a further embodiment, the present invention provides a ligand capable of identification of tse-infected T-cells, characterized in that specific interaction between said ligand and said tse-infected T-cell is based on the infectivity of said T-cell.

In a further embodiment, the present invention provides the use of a ligand as above in a method of analysis of said tse-infected T-cell.

Further embodiments of the present invention are set out in the dependent claims.

### DETAILED DESCRIPTION OF THE INVENTION

The present invention involves detailed investigations about the nature of the limiting factors and/or physical entities in the development of spongiform encephalopathy after peripheral infection. Thus, the present invention involves identification of the physical carriers of prions and of the mechanisms involved in the spread of infectivity.

### DEFINITIONS

As referred to in the present application, the term prion designates the agent of transmissible spongiform encephalopathy (tse).

As referred to in the present application, the term PrP^{c} designates the naturally occurring form of the mature PrnP gene product. Its presence in a given cell type is necessary, but not sufficient, for replication of the prion.

As referred to in the present application, the term PrP^{Sc} designates an “abnormal” form of the mature PrnP gene product found in tissues of tse sufferers, defined as being partly resistant to digestion by proteinase K under standardized conditions. It is believed to differ from PrP^{c} only (or mainly) conformationally, and is considered to be the transmissible agent or prion.

As referred to in the present application, B-cells (or B-lymphocytes) are to be understood as members of a subset of lymphocytic cells which are precursors of plasma cells which produce antibodies; they are able to recognize free antigens and antigens located on cells.

As referred to in the present application, T-cells (or T-lymphocytes) are to be understood as members of a subset of lymphocytic cells responsible for cellular immunity and the production of immunomodulating substances.

As referred to in the present application, the term ‘lymphocytes’ designates cells which participate in the humoral and cell-mediated immune defense, and which accordingly comprise B-cells and T-cells.

As referred to in the present application, the term ‘animals’ encompasses all eukaryotic organisms excluding plants.

### FIGURES

FIG. 1 shows the brain histopathology of immune deficient and control mice after i.p. inoculation of scrapie prions. The hippocampal formation was immunostained for glial fibrillary acidic protein, and identical segments of the pyramidal cell ribbon were microphotographed (200×). Intense, diffuse gliosis was visible in brains of T-cell-deficient, SCID, TNF-r1^{o/o}, t11 μMT, and infected control mice. Some rag-2^{0/0} and μMT mice showed spongiform encephalopathy, but others of the same genotype did not display any pathology after similar time periods following i.p. inoculation, and were indistinguishable from mock-infected C57BL/6 mice.

FIG. 2 relates to the Western blot analysis of brains of immune-deficient mice after i.p. inoculation with transmissible spongiform encephalopathy prions and lack of specific antibodies against PrP in t11 μMT mice. FIGS. a,b are-Western blots of brain material electrophoresed native (−) or after digestion with proteinase K (PK) (+). Large amounts of PK-resistant prion protein (PrP^{sc}) were detected in all mice that had developed spongiform encephalopathy, as well as in one agr^{o/o} (a) two rag-2^{o/o} and two μMT mice (b). One further B-cell-deficient mouse proved negative for PrP^{sc} (not shown), and no clinical symptoms of spongiform encephalopathy were detected in any B-cell-deficient mice irrespective of accumulation of PrP^{sc}. FIG. c shows a Western blot prepared: with recombinant murine PrP from E. coli (PrP^{R}), total brain protein extract from a wild-type mouse (WT), and total brain protein extract from a Prnp^{o/o} mouse (0/0)¹⁵. Blots were incubated with serum from a t11 μMT mouse inoculated with prions i.p. (left), stripped and reprobed with monoclonal antibody 6H4 to recombinant PrP (right). The presence of PrP-specific antibodies, as indicated by a 20 K band in lane PrP^{R} and by a cluster of bands present in lane WT but absent from lane 0/0, is evidence with 6H4 antibody but undetectable in t11 μMT serum. Relative molecular mass markers (top to bottom): 105 K, 82 K, 45 K, 37.3 K, 28.6 K, 19.4 K. FIG. d shows the FACS analysis of immunoreactivity of t11 μMT serum. Ordinate: cell counts; abscissa: logarithm of fluorescence intensity. Serum from a t11 μMT mouse 210 days after i.p. inoculation with prions was diluted 1:10 and 1:100, stained VSV-infected EL4 cells (top panel, unfilled area) almost as strongly as VSV-specific monoclonal antibody VI24 (filled area). In contrast, immunoreaction of t11 μMT serum (1:10) with CD3⁺ T-cells from C57BL/6, tga20, tg33 (ref.29) and Prnp^{o/o} mice (lower panels) did not exceed background, like normal C57BL/6 serum on EL4 cells (top panel, dotted line). The same profiles were obtained when probes were stained with serum of untreated t11 μMT mice (data not shown).

FIGS. 3a and 3b display a flow analysis printout showing enriched B-cell and T-cell populations.

FIG. 3b shows again flow cytometric analysis of splenocytes and of purified splenocyte fractions, however also the non B/T-cell fraction is shown as third constituent. Splenocytes from wild-type mice 34 days after i.p. inoculation with RML scrapie agent were fractionated as described in the experimental section and subjected to FACS analysis. More than 99% of the cells in the purified B-cell fraction were positive for the mouse B-cell marker B220 and negative for the mouse T-cell marker CD3. Similarly, more than 99% of the purified, cells in the T-cell fraction were positive for CD3 and negative for B220. The same results were obtained whether or not the cells were gated for lymphocytes by forward and side scattering. Ordinate: cell counts; abscissa: logarithm of fluorescence intensity.

FIG. 4a Shows the infectivity of splenocytes in Wild type mice and Spleen mice on a linear scale.

FIG. 4b shows a further comparison of the infectivity of splenocytes in Wild type mice and Spleen mice at a different time point and on a logarithmic scale. Serial 10-fold dilutions of splenocytes & splenocyte fractions were inoculated intracerebrally into groups of four indicator mice and incubation time to terminal scrapie disease was determined. Infectivity titers were calculated by the end point titration method (according to Reed, J. Muench, H. A. A simple method of estimating fifty percent endpoints. Am. J. Hygiene 27, 493-497 (1938)), assuming 3×10 lymphocytes/spleen, of which 65% were B-lymphocytes and 3 5% T-lymphocytes. The detection limit of the infectivity assay corresponds to 100 LD₅₀ units per spleen.

FIGS. 5a-5c show the infectivity in different cell types of Wild type mice, T-cell mice and Spleen mice.

FIG. 6 is a schematic representation of half-genomic PrP transgenes driven by heterologous promoters. The genomic mouse Prnp locus is shown on top (Westaway, D., Cooper, C, Turner, S., Da, C. M., Carlson, G. A. and Prusiner, S. B. (1994) Structure and polymorphism of the mouse prion protein gene. Proc. Natl. Acad. Sci. USA 91, 6418-22). Construction of the ‘half-genomic’ PrP vector (phgPrP) lacking the 12-kb intron 2 has been described (Fischer, M., Rülicke, T., Raeber, A., Saller, A., Moser, M., Oesch, B., Brandner, S., Aguzzi, A. and Weissmann, C. (1996) Prion protein (PrP) with amino-proximal deletions restoring susceptibility of PrP knockout mice to scrapie. EMBO J. 15, 1255-1264). Using PCR with the primers PE1 and Del, a BamHI site was introduced at the 5′ end of exon 1 in phgPrP. The resulting promoterless construct pPrP-5′HG EcoRI was cloned into Bluescript, the PrP sequence was extended up to the Sall site in the 3′ non-coding region by introducing the Narl-Sall fragment of phgPrP to yield pPrP-5′HG Sall. Promoter cassettes were inserted into the BamHI site of pPrP-5′HG Sall to yield plck-PrP-5′HG Sall, pEμ/IRF1-PrP-5′HG Sall and pAlbumin-PrP-5′HG Sall. B, BamHI; K, Kpnl; N, Narl; Nt, Notl; R, EcoRI; S, Sall; X, Xbal. Wavy lines, vector sequences.

FIG. 7 is a Northern blot analysis of PrP RNA in organs of various mouse lines. Total RNA (10 μg) was electrophoresed through an agarose gel and blotted onto filters. The filters were hybridized with a PrP ORF probe (PrP), stripped and re-hybridized with a glyceraldehyde-3-phosphate dehydrogenase (GAPDH) probe. Blots of Prnp^{+/+}, Prnp^{o/o}, Tg94/IRF and Tg33/Ick tissues hybridized with the ³²P-labeled PrP probe were exposed for 1 d. Longer exposure of the blot (not shown) revealed faint signals in Tg33/Ick brain, lung and intestine. Blots of Tg01/alb tissues hybridized with the PrP probe were exposed for 8 d. Blots rehybridized with the GAPDH probe were exposed for 20 h. Radioactivity was quantitated on a phosphorimager; the values represent the PrP signals (inasmuch as they were significant) relative to the GAPDH signal. Positions of the 28S and 18S ribosomal RNAs are shown on the right.

FIG. 8 is an analysis of PrP expression by FACS and immunohistochemistry. FACS analysis for cell surface PrP was carried out on splenocytes (A), thymocytes (B) and peripheral blood leukocytes (PBL) gated for lymphocytes (C) form Prnp^{+/+}, Prnp^{o/o}, Tg94/IRF and Tg33/Ick mice. Cells were stained with anti-PrP polyclonal antisera R340 and phycoerythrin-conjugated anti-rabbit IgG and analyzed by FACS gated for lymphocytes. For two-colour FACS analysis (A), PrP staining was followed by B cell staining with FITC-conjugated anti-B220 antibodies or T-cell staining with FITC-conjugated anti-CD3 antibodies. (D) Double immunofluorescence analysis of splenic germinal centers in noninoculated Tg94/IRF (a-d), wild-type mice (e-h), and Prnp^{o/o} mice (j-m). Sections were stained with haemalaun (a, e, j), with peanut agglutinin (PNA) (green; b, f, k), and with antiserum R340 to PrP (red; c, g, 1). The majority of PNA-labeled in the germinal center B-cells were PrP-positive in Tg94/IRF mice (d; yellow signal in superimposed images) and in wild-type mice (h), but PrP-negative in Prnp^{o/o} mice (m). Original magnification 250×. (E) Immunofluorescence labeling of follicular dendritic cells and PrP on consecutive sections of spleen from non-inoculated Tg94/IRF (a-d), Tg33(Ick (e-h), wild-type mice (j-m) and Prnp^{o/o} mice (n-q). Sections were stained with haemalaun (a, e, j, n), antibody FDC-M1 to follicular dendritic cell (green; b, f, k, o), antiserum R340 to PrP (red; c, g, 1, p) and rabbit pre-immune serum (PIS) (d, h, m, q). In wild-type spleens (k, l), PrP was stained exclusively in the germinal centers, most strongly in the areas also stained by FDC-M1. In Tg94/IRF mice (b, c), PrP was evenly distributed over the entire section, including the region also stained by FDC-M1. In Tg33/Ick spleens, PrP was visualized mainly in the T cell areas but some cells were stained in the region also stained by FDC-M1. No PrP staining above background (q) was found in germinal centers of Prnp^{o/o} mice (p). Original magnification 250×.

FIG. 9 is an immunoblot analysis for PrP in tissues of various mouse lines. A. Aliquots (120 μg protein) of tissue homogenates as indicated were loaded per lane. B. Aliquots (40 μg protein) of tissue homogenates were digested with 500 units of PNGaseF for 2 h at 37° C. C. Aliquots of tissue homogenates as indicated were immunoprecipitated with 6H4 antibody coupled to Sepharose. The eluted proteins were subjected to Western blotting and PrP was detected on blots with 1:10,000 diluted polyclonal anti-PrP antiserum 1B3. Molecular weight markers are indicated on the left in kD.

FIG. 10 shows a Western blot (antibody 6H4) carried out directly with spleen cells, B-cells, T-cells and non B-/T-cells of i.p. infected wild type without prior passage through indicator mice. Though the PrP^{Sc} level (lanes PK +) at an early time point is too low to detectably appear in the spleen cells, its presence in B and T-cells and its absence in the non B-/T-cell fraction is clearly apparent.

FIG. 11 is a FACS analysis of PBLs from animals before and during treatment with Dexamethasone and Cyclophosphamide. B-cells were detected with a FITC labeled α-CD19 antibody while T-cells were monitored with a PE-labeled α-CD3 antibody. Thirty ml of blood were assayed for every sample counting all events for a defined period of time. FACS data clearly demonstrate a drastic decrease in fluorescence signals for B and T-cells at both time points tested.

FIG. 12 is an ELISA analysis of serum from experimental animals at different time points after first depletion. Serum was diluted and bound to plates coated with α-IgM or -IgG antibodies. Binding of serum antibodies to plates was monitored with HRP conjugated α-Immunoglobulin antibodies. Data show a clear decrease of serum IgM and IgG levels starting with a delay of 14 (IgM) to 28 (IgG) days. 48d after depletion, levels are comparable to the μMT control. 84 days after start of depletion which is 16 days after stop of therapy.

FIG. 13 is a Western blot analysis of spleen homogenates from infected animals with & without depletion of B- and T-cells. Proteinase K digestion of samples reveals accumulation of resistant material only in control animals. No resistant material was detectable in spleen homogenates from animals treated with Cyclophosphamide and Dexamethasone.

FIG. 14 shows schematically the development of infectivity in spleen of PrnP^{-/-}, Prnp^{+/+} and drug treated Prnp^{+/+} (onset of treatment 10 after i.p. inoculation)

### INVESTIGATIONS CARRIED OUT BY THE INVENTORS

### The Role of the B-Cells

As apparent from the prior art, the development of neurological disease after peripheral infection with transmissible spongiform encephalopathy depends on abnormal prion expansion within the cells of the lymphoreticular system^{3,4}. The skilled man is however aware that the immune system comprises several components whose identity and precise function and specific interaction with the remaining components are still the object of extensive scientific investigation. Among the immunocompetent and other components of LRS, at least stem cells, plasma cells, NK cells, B-cells, T-cells, dendritic cells, eosinophiles, basophiles, monocytes, macrophages, reticular cells, capillary sheath cells, polymorphonuclear neutrophils, mast cells are referred to in the literature, but even this is a non-exhaustive listing. Moreover, mutual interaction of these and further components of the LRS is rendered extremely complicated because of the dependency on the maturational stage of each component involved. The inventors have investigated here for the first time the roles of different components of the immune system by using a panel of immune-deficient mice inoculated with prions intraperitoneally and found that defects affecting only T-cells had no apparent effect, but that all mutations that disrupted the differentiation and response of B-cells prevented the development of clinical spongiform encephalopathy. As an absence of B-cells and of antibodies correlates with severe defects in follicular dendritic cells, a lack of any of these three components may prevent the development of clinical spongiform encephalopathy. The key function of the follicular dendritic cells has been postulated inter alia by Muramoto, vide supra. However, the inventors found surprisingly that spongiform encephalopathy developed after peripheral inoculation in mice expressing immunoglobulins that were exclusively of the M subclass and without detectable specificity for the normal form of the prion PrP^{c}, and in mice which had B-cells but no functional follicular dendritic cells. Thus, the inventors have found out that differentiated B-cells are crucial for neuroinvasion by spongiform encephalopathy, regardless of the specificity of their receptors.

The effect of combined immune defects on the pathogenesis of spongiform encephalopathy was studied in mice deficient in rag-2 (ref. 5) and rag-1 (ref. 6), which lack B and T-cells, in scid (severe combined immune deficient) mice, and in agr^{o/o} mice, which lack rag-2 as well as the receptors for interferon-α/β⁷ and interferon-7⁸. Such mice were obtained according to methods well-known in the art of genetic engineering. For controls, inbred mice of strains C57BL/6 and 129/Sv which are the genetic backgrounds of all other mouse strains used were inoculated as well. To investigate the role of T-cells, the inventors used mice with targeted disruption of the genes encoding CD4 (ref. 9), CD8 (ref. 10), β₂-microglobulin¹¹ or perforin¹². Selective depletion of B-cells was studied in μMT mice¹³, which have a targeted disruption of the transmembrane exon of the immunoglobulin μ-chain gene, do not produce any immunoglobulins and suffer from a B-cell differentiation block at the large-to-small pre-B-cell transition, yet bear complete and functional T-cell subsets.

After intracerebral (i.c.) challenge with prions, all immune-deficient mice developed clinical symptoms of spongiform encephalopathy. This was confirmed by histopathological analysis (not shown) and by transmission of disease to indicator tga20 mice, which over-express the normal prion protein (PrP^{c}) and are hypersensitive to spongiform encephalopathy¹⁴ (Table 1). Transmission to Prnp^{o/o} mice¹⁵, which do not express PrP^{c} and are resistant to spongiform encephalopathy¹⁶ (n=4), did not induce disease after >210 days, as expected for bona fide spongiform encephalopathy. In all groups, latency times from inoculation’ to first appearance of clinical symptoms and to terminal disease. (Table 2), as well as brain prion infectivity titres (Table 1), were similar to those of control mice.

Thus, if prions where delivered to the central nervous system, spongiform encephalopathy pathogenesis and prion expansion in the brain proceeded without any detectable influence of the immune status of the host.

When mice were exposed to prions through the intraperitoneal (i.p.) route, mice homozygous-null for CD4, CD8, β₂-microglobulin or perforin developed the initial symptoms of disease and terminal spongiform encephalopathy with latency periods similar to those of C57BL/6 and 129/Sv mice (Table 2), and reached analogous prion titres in both spleen and brain (Table 1). Thus the inventors concluded that CD8⁺ cytotoxic and CD4⁺ helper T-cells are not rate-limiting for spongiform encephalopathy after peripheral inoculation of prions, in agreement with the observation that nude mice develop spongiform encephalopathy normally after i.p. inoculation³.

In contrast, no disease appeared after i.p. inoculation in μMT and in rag-deficient (rag-7^{o/o}, rag-2^{o/o} and agr^{o/o}) mice, and no prion infectivity was detectable in their spleens (Table 1). In SCID C57BL/6 mice, disease was marginally prolonged, which disagrees with earlier results^{4,17} and may be due to incomplete immune deficiency of SCID mice in specific genetic backgrounds^{18,19}, because SCID C.B-17 mice (whose immune defect is less leaky] did not develop disease (Table 2). Also, it should be borne in mind that B-cell differentiation to immune competence exhibits redundancy at many points; that renders such cells only partially sensitive to genetic manipulation.

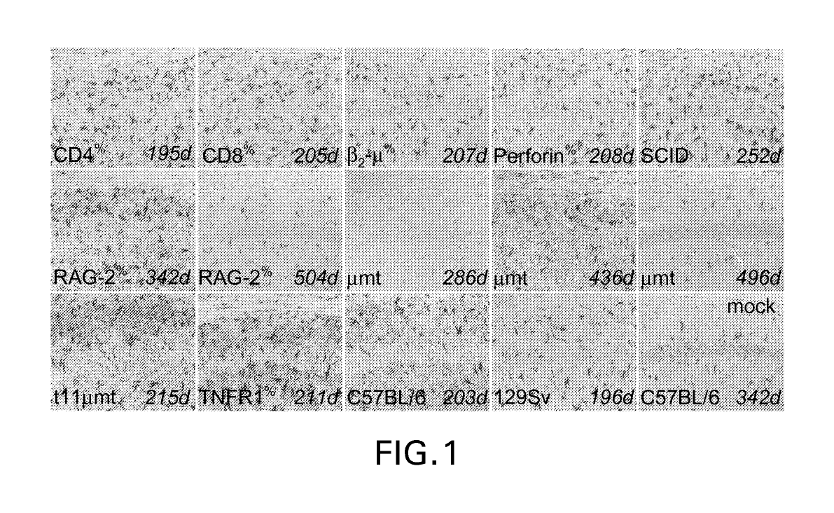

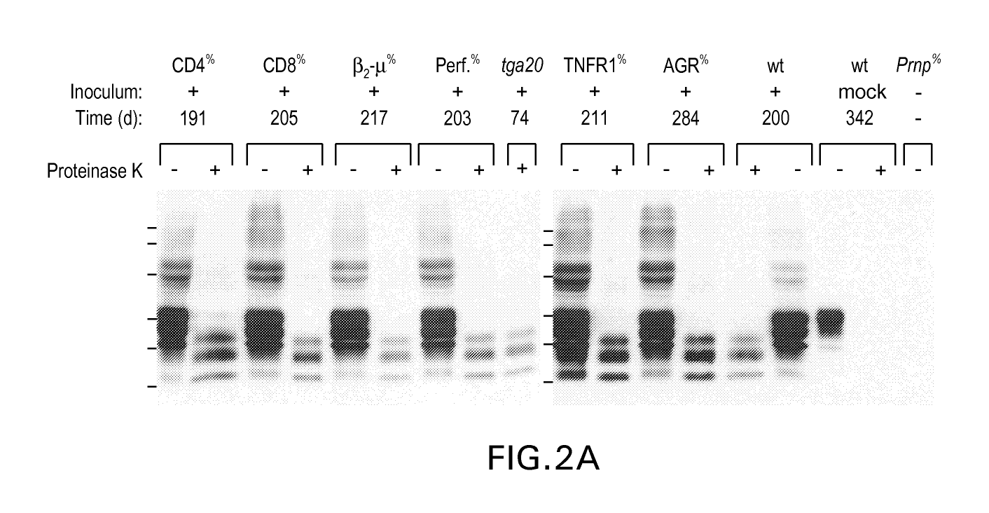

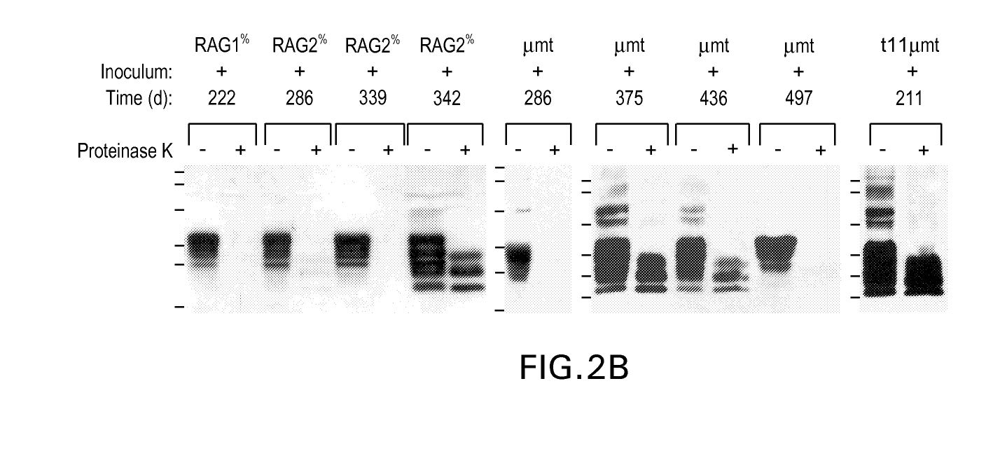

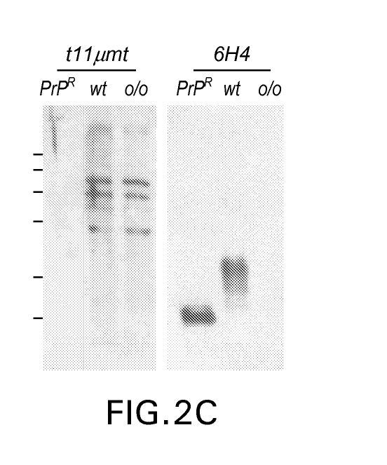

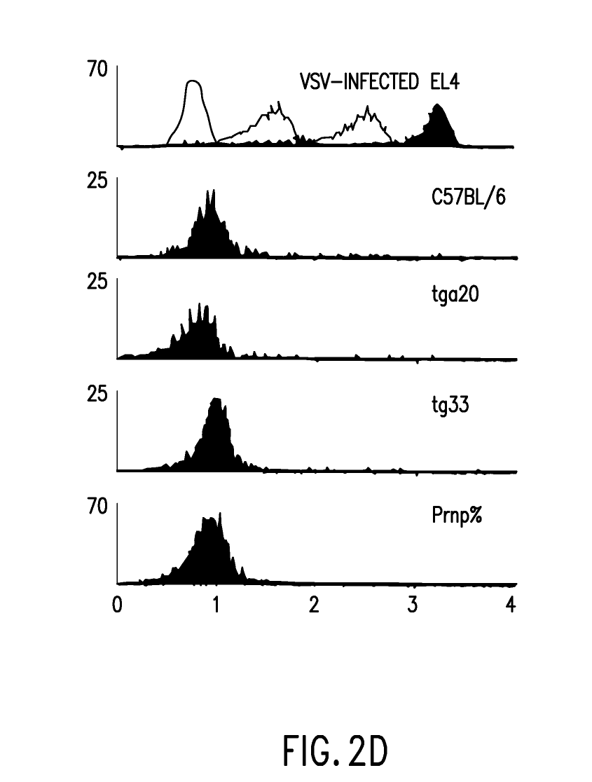

Histopathological examination of brain sections revealed generalized spongiform encephalopathy in all wild-type and immune-deficient mice clinically diagnosed as spongiform encephalopathy-sick (FIG. 1). In addition, and despite lack of clinical symptoms, spongiform encephalopathy was seen in 1/7 rag-deficient and ⅙ μMT mice (at random sampling) 342 and 436 days after i.p. inoculation (FIG. 1), and significant prion titres were found in brains of 3/7 rag-deficient mice and ⅓ μMT mice (Table 1). Western blot analysis revealed accumulation of the disease ‘form of prion, PrP^{sc}, in the brains of 2/6 rag-deficient and 2/6 μMT mice inoculated i.p. (FIG. 2). The remaining rag-deficient and μMT mice did not accumulate PrP^{sc} as late as 504 days after inoculation.

For the sake of absolute scrutiny, it may thus be concluded that the latter findings are compatible with incipient spongiform encephalopathy in a minor fraction of B-cell-deficient mice. Therefore, although it prevents ‘neuroinvasion’ of the spongiform encephalopathy agent in most cases, absence of B-cells uncovers a slower, <50% efficient mechanism of pathogenesis which may cause spongiform encephalopathy in situations of immune deficiency. It should be emphasized that even then, B-cell deficiency prolongs the delay between PrP^{sc} accumulation, onset of spongiform encephalopathy histopathology and clinical symptoms beyond the typical life expectancy of mice.

These results suggest that B-cells may ‘transport’ prions from lymphoid organs to nervous tissue. Alternatively, the apparent protection of B-cell-deficient mice from prions administered i.p. may result from the absence of immunoglobulins. Complexing of PrP^{sc} with antibodies may favour nucleation (a process proposed to underlie the formation of prion infectivity 2) or may opsonize PrP^{sc} and enhance access to lymphoid sites of abnormal prion expansion. It also may suggest that animals become more able to propagate infection if the genetic change is later in B-cell development. To clarify this question, the inventors inoculated t11 μMT mice (μMT mice expressing a rearranged IgM transgene directed against the glycoprotein of vesicular stomatitis virus) and found that they could support normal B-cell differentiation but exclusively expressed the transgenic IgM heavy chain, had a heavily skewed and very limited antibody repertoire, and lacked immunoglobulins of the D, G, E and A subclasses. Such mice were obtained according to methods well-known in the art.

After i.p. inoculation with prions, t11 μMT mice developed disease with a latency comparable to that of wild-type mice (Table 2) and accumulated PrP^{sc} in their brains (FIG. 2b). Serum from both uninfected and terminally spongiform encephalopathy-sick t11 μMT mice inoculated i.p. was shown by western blotting and by flow-assisted cell sorting (FACS) analysis not to cross-react with PrP^{c} (FIG. 2c, d), suggesting that IgGs are not the effectors of prion ‘neuroinvasion’, and that a specific humoral immune response (at least as assessed by FACS and western-blot analysis) cannot be correlated with peripheral pathogenesis of spongiform encephalopathy. However, for the sake of absolute scrutiny, one cannot exclude the possibility that IgMs below the threshold of detectability, or indirect effects of antibodies, may be involved in spongiform encephalopathy pathogenesis. This corresponds to the difficulty in obtaining reliable disease transmission from soluble serum components from diseased animals.

B-cells are required for maturation of follicular dendritic cells (FDCs) and formation of germinal centres. Protection of B-cell-deficient mice may therefore result from the absence of FDCs, especially as FDCs accumulate PrP^{Sc} extensively in i.p.-inoculated mice³ and in the tonsils of patients suffering from new variant CJD²¹. Thus, the inventors inoculated mice lacking tumour-necrosis factor receptor-1 (TNF-R1^{o/o})²², which have virtually no germinal centres in lymphatic organs and very few, if any, FDCs²³, despite differentiation of functional B- and T-cells. These mice developed spongiform encephalopathy after both i.c. and i.p. inoculation, as did control mice (Table 2), thus disproving a prime role for FDCs in peripheral pathogenesis and supporting the inventors' previous results that adoptive transfer of fetal liver cells (which does not efficiently replace FDCs²⁴) can restore high spleen prion titres after i.p. inoculation²⁵.

TABLE 1
Scrapie symptoms, scrapie histopathology
and infectivity titres in immunodeficient mice

| | Primary infection (route of inoculation: i.c.) | | | Infectivity bioassay | | | |
|---|---|---|---|---|---|---|---|
| Genotype | Incubation (d) | Symptoms | Pathology | Brain | | Spleen | |
| CD-4^{0/0} | 176 | + | + | 7.2* | (65 d ± 2†) | 6.6 | (71 d ± 2) |
| CD-4^{0/0} | 154 | + | + | 7.4 | (63 d ± 1) | 7 | (67 d ± 2) |
| scid | 167 | + | + | 7.3 | (64 d ± 1) | 5.5 | (81 d ± 2) |
| scid | 167 | + | + | 7.6 | (62 d ± 2) | 5.2 | (83 d ± 1) |
| rag-2^{0/0} | 171 | + | + | 7.3 | (64.5 d ± 2) | <0 | (>200 d) |
| agr^{0/0} | 182 | + | + | 7.3 | (64.5 d ± 1) | <0 | (>145 d) |
| μMT | 175 | + | + | 7.9 | (59 d ± 5) | <0 | (>200 d) |
| | Primary infection (route of inoculation: i.p.) | | | Infectivity bioassay | | | |
| CD-4^{0/0} | 191 | + | + | 7.3 | (64 d ± 1†) | ND | |
| CD-4^{0/0} | 195 | + | + | 7.5 | (62.5 d ± 2) | ND | |
| scid | 214 | + | + | 7.3 | (64 d ± 1) | 5.2 | (83 d ± 1) |
| scid | 249 | + | + | 7.7 | (61 d ± 2) | 5 | (85 d ± 1) |
| rag-2^{0/0} | 286 | − | + | 6.5 | (72 d ± 2) | <0 | (>200 d) |
| rag-2^{0/0} | 286 | − | − | <0 | (>200 d) | <0 | (>200 d) |
| rag-2^{0/0} | 339 | − | − | <1 | (>122 d) | <2 | (>115 d) |
| rag-2^{0/0} | 342 | − | + | 7.5 | (65 d ± 1) | <2 | (>115 d) |
| rag-1^{0/0} | 222 | − | − | <1 | (>122 d) | <2 | (>115 d) |
| agr⁰ | 284 | − | + | 7.2 | (65 d ± 0) | <0 | (>139 d) |
| agr^{0/0} | 349 | − | − | <0 | (>139 d) | <0 | (>139 d) |
| μMT | 286 | − | − | <0 | (>200 d) | <0 | (>200 d) |
| μMT | 286 | − | − | <0 | (>200 d) | <0 | (>200 d) |
| μMT | 375 | − | − | 7.8 | (60 d ± 1) | <0 | (>200 d) |

| μMT | 436 | − | + | ND | | ND |
|---|---|---|---|---|---|---|
| TNF-R1^{0/0} | 211 | + | + | 7.7 | (61 d ± 1) | ND |
| TNF-R1^{0/0} | 212 | + | + | 7.7 | (60 d ± 1) | ND |

For infectivity bioassays. brain or spleen homogenates were injected intracerebrally into groups of four tga20 mice. ND, not determined.
*prion titres expressed as log LD₅₀ per g of spleen or brain tissue.
†incubation time, in days, of indicator tga20 mice (average ± standard deviation).

TABLE 2
Latency of scrapie In different Immunodeficient mice

| | | Intracerebral route | | Intraperitoneal route | |
|---|---|---|---|---|---|
| Defect | Genotype | Scrapie | Time to terminal disease (d) | Scrapie | Time to terminal disease (d) |
| T | CD-4^{0/0}* | 7/7 | 159 ± 11 | 8/8 | 191 ± 1 |
| T | CD-8^{0/0}* | 6/6 | 157 ± 15 | 6/6 | 202 ± 5 |
| T | β₂-μ^{0/0}* | 8/8 | 162 ± 11 | 7/7 | 211 ± 6 |
| T | Perforin^{0/0}* | 3/4† | 171 ± 2 | 4/4 | 204 ± 3 |
| T and B | SCID* | 7/8† | 160 ± 11 | 6/8‡ | 226 ± 15 |
| T and B | SCID§ | 4/4 | 152 ± 1 | 1/4∥ | 289 |
| T and B | rag-2^{0/0}* | 7/7 | 167 ± 2 | 0/7 | Healthy (>504) |
| T and B | rag-1^{0/0}* | 3/3 | 175 ± 2 | 0/5 | Healthy (>258) |
| T and B | agr^{0/0} [symbol] | 6/6 | 184 ± 10 | 0/7 | Healthy (>450) |
| B | μMT* | 8/8 | 181 ± 6 | 0/8 | Healthy (>534) |
| IgG | t11 μMT* | 5/5 | 170 ± 3 | 4/4 | 223 ± 2 |
| FDC | TNF-R1^{0/0}# | 7/7 | 165 ± 3 | 9/9 | 216 ± 4 |
| Controls | 129Sv | 4/4 | 167 ± 9 | 4/4 | 193 ± 3 |
| | C57BL/6 | 4/4 | 166 ± 2 | 4/4 | 206 ± 2 |

All mice developed spongilorm encephalopathy after i.c. inoculation. In contrast, B-cell-deficient mice stayed healthy after i.p. inoculation of RML scrapie prions.
*Genetic background was inbred C57BL/6.
†One Perforin^{0/0} and one SCID mouse suffered from intercurrent death 135 and 141 days after inoculation, respectively.
‡Two SCID C57BL/6 mice remained healthy and were killed 303 and 323 days after inoculation.
§Genetic background was inbred C.B-17.
∥¾ SCID C.B-17 mice remained healthy (>340 d). Four further SCID C.B-17 mice were challenged with 100 μl of a 10⁻¹ dilution of RML prions, and all remained healthy (>340 d). [symbol]
Genetic background was C57BL/6 × 129Sv.
#Genetic background was inbred 129Sv.

Thus, the inventors have identified B-cells and B-cell-dependent processes as a limiting factor in the development of transmissible spongiform encephalopathy after peripheral infection. It appears therefore that tse-infected (i.e. PrP^{Sc} carrying) B-cells are the bottle neck of disease promulgation. Accordingly, the present invention provides a novel, specific and therefore more preferable procedure to advantageously selectively suppress that component of the immune system which is responsible for the prion spread, namely the B-cells.

### The Role of the T-Cells

Still further, the inventors have studied the role of B-cell-dependent processes during pathogenesis. Accordingly, the inventors have carried out further experiments aiming at establishing the amount and nature of possible interaction of tse-infected B-cells with the remaining components of the immune system, e.g. with T-cells. Results of the inquiry about such interaction and design of suitable therapeutic measures influencing such interaction are a further aspect influencing the present invention.

As pointed out above, it is known that mice devoid of functional PrP genes (Prn-p^{o/o}) are resistant to transmissible spongiform encephalopathy and do not propagate prions (Büeler et al. Cell, 73, 1339-1347, 1993). Thus, reintroduction of PrP transgenes into Prn-p^{o/o} should restore transmissible spongiform encephalopathy. Departing from this concept, the inventors conducted studies in Prn-p^{o/o} mice transgenic for PrP genes controlled by tissue specific promoters. Such mice may be obtained by the man skilled in genetic engineering according to methods well-known in the art. Specifically, the inventors used ‘T-cell mice’ (Ick promoter; Chaffin et al. (1990), EMBO J. 9, 3821-3829) which express PrP exclusively in T-cells and ‘spleen mice’ designated tg94/IRF (IRF-1 promoter/Eu enhancer; Yamada et al., Proc. Natl. Acad. Sci. USA. 88, 532-536, 1991) which express PrP in splenocytes and at low level in brain. Challenge of spleen-mice with prions led to the development of spongiform encephalopathy in that spleen mice succumbed at a late stage due to brain disease and showed propagation of prions in spleen and thymus as well as in brain. On the other hand, T-cell mice showed no propagation of prions. Accordingly, these results are fully consistent with the prior experiments as described hereinabove and confirm the crucial role of B-cells (Table 3).

TABLE 3
Table 1: Transmission of mouse prions to transgenic
mice with ectopic PrP expression

| | Incubation time | |
|---|---|---|
| Mice | Mean Days ± sd | n/no |
| Prn-p^{+/+} | 196 ± 4 | 10/10 |
| Prn-p^{0/0} | >500 | 0/3 |
| “Spleen mice” | 263 ± 5 | 7/13 |
| “T cell mice” | >500 | 0/5 |

To further investigate the role of B-cell dependent processes, in a second step, the infectivity of the splenocytes from spleen mice was selectively determined in a bioassay. It was found that, though most of the infectivity was indeed carried by the B-cells, the T-cells were also contaminated to some extent (Table 4).

TABLE 4
Table 4: Infectivity of total and fractionated splenocytes from “Spleen
mice” 120 days after i.p. inoculation with prions. Cells were fractionated
by magnetic activated cell sorting (MACS) using anti-B220 antibodies
for B cells and anti-Thy 1.2 antibodies for T-cells.

| Cell fraction | Titer (LD50 units/10⁶ cells) |
|---|---|
| splenocytes | −200 |
| B cells | −500 |
| T cells | −100 |
| non-B, non-T cells | <1 |

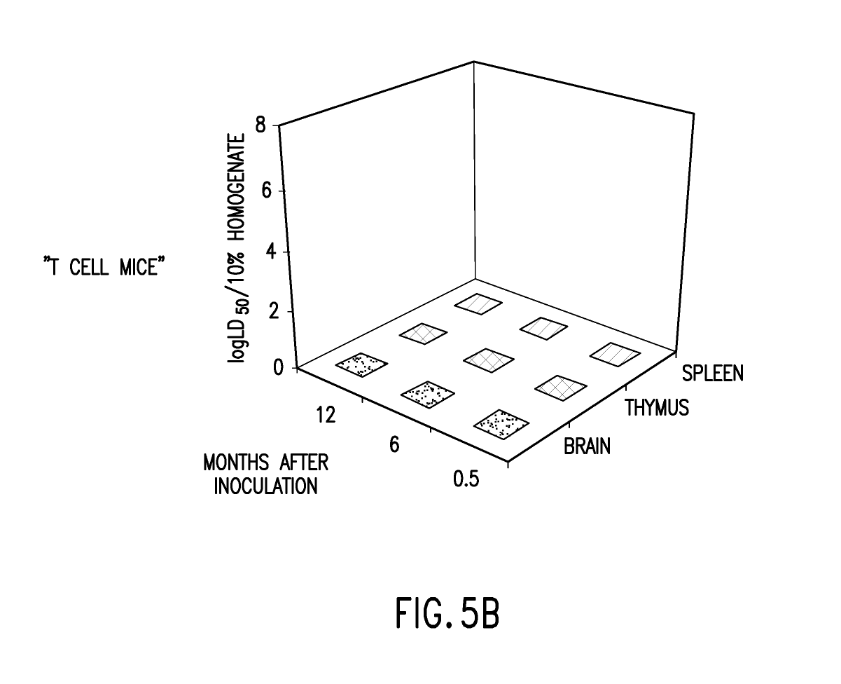

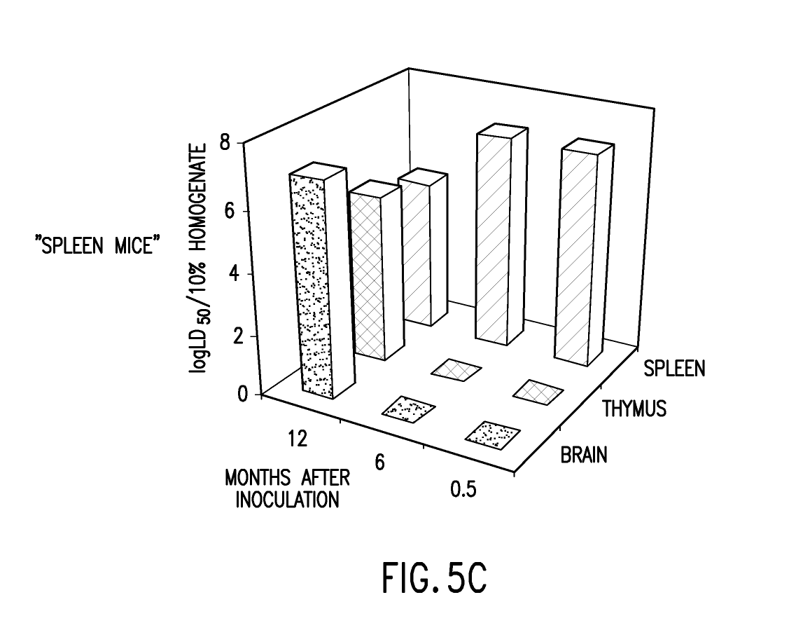

This newly found contamination shown by the T-cells of the spleen mice seems to be in contradiction with the fact that the T-cells of T-cell mice do not show any contamination (see FIG. 5a-c). However, what initially, seems to be a contradiction (infectivity of T-cells in some cases, non-infectivity of T-cells in other cases), in reality implies and supports the existence of an interaction between the B-cells (the carriers of infectivity) and the T-cells (which, as such, are not able to propagate infectivity). Spleen mice contain both T-cells' and B-cells, and upon infection of the B-cells, a B-cell mediated secondary infection of the spleen mice's T-cells takes place (see e.g. Table 6). On the contrary, T-cell mice do not contain PrP expressing B-cells, and as a consequence of this lack of infectivity carriers, the T-cell mice's T-cells are not subject to infection. Thus, depending on the extent of disease progress within an infected host undergoing therapy, provision of T-cell depletants for the treatment of transmissible spongiform encephalopathy is a further aspect of the invention.

### CONCLUSIONS

As pointed out above, not only the crucial carrier of infectivity, namely the B-cells, has been identified, but also a powerful tool for the monitoring of the spread of transmissible spongiform encephalopathy within the immune system of an infected human or animal has been provided for the first time by the inventors. Indeed, the present invention allows the distinction between the occurrence of tse-infected B-cells alone and the further occurrence of secondarily tse-infected T-cells. Accordingly, a further aspect of the invention is also the testing of the effectivity of medicaments by assay methods capable of monitoring the spread of transmissible spongiform encephalopathy within the immune system after administration of such medicaments. Such an assay contemplates the monitoring of biological or biochemical•parameters of B-cells and T-cells to determine the occurrence of secondary infection as an indicator of the disease progress.

In particular, the above finding that removal of B-lymphocytes by surface antigen B-cell autolysis limits or prevents the transmission of prion disease infection demonstrates that the absence of such cellular components prevents transfer of infectivity to other cells, such as T-i cells, or development of disease. It follows logically that tse-infected B-cells are predictive of the pathological outcome and, progression of prion disease. These disease specific components of the cellular immune system can then effectively stage developing disease or predict the status of disease in an individual organism undergoing treatment.

For conducting further studies, B and/or T-lymphocytes are isolated from blood by standard techniques known to preserve phenotypic cellular features. Cells isolated in this manner may be evaluated without manipulation or fixed by suitable methods and then introduced into liquids solutions composed of well know constituents containing binding partners or antibodies characteristic for cells that may express “prion disease” phenotypic determinants or classical lymphocyte determinants distributed among progenitor and/or daughter cells of a given developmental lineage in way characteristic of the disease. These components may be selected from but not limited to cellular differentiation, CD, antigens such as CD 19, CD 20, etc and/or binding partners specific for certain intracellular or extra cellular disease specific cellular phenotypes such as antibodies to normal or abnormal prion proteins. These components may be disease strain or species specific. These phenotypes or distribution of phenotypes correlate with the infectivity or the transmission of infectivity. It is to be realized that such CD or prion disease specific antigens, or determinants may be differentially distributed in qualitative or quantitative manner among lymphocytes of different stages of development and functional lineages. The relationship of such phenotypic determinants in cell populations is diagnostic of the presence of disease, the presence of disease progress in advancing or the degree of regression of disease undergoing treatment, depending on the status of the organism in question.

Since the unusual and novel observation that B-cells provide the necessary germinal site for the disease promulgation, the analysis of specific determinants in B and T lymphocytes will provide insight into the disease progression. It is to be understood that the means of detecting these proportional relationships of cells among different phenotypic populations could be achieved by means of histopathological methods or automated flow cytometric methods utilizing sophisticated data analysis algorithms to display results in a readily interpretable way.

The skilled reader will also appreciate that the finding that the route of infection is based on the interaction between the prions and the B-cells and the T-cells is indeed a revolutionary achievement which could not have been reached if one would have pursued the path indicated by the background art in the field. Keeping in mind the accredited notions of all previous research, it is clear that the findings of the present inventors are original and that they derive from a very sophisticated scientific approach.

Indeed, back in 1967, it was still found and believed (see Eklund et al. Pathogenesis of scrapie virus infection in the mouse. J. Infectious Diseases 117, 15-22 (1967)) that the infective agent was of the viral kind. No specific indication of the role (if any) of B and T-cells could be derived from the teachings of Eklund et al.

Later, while Cashman et al. (Cellular isoform of the scrapie agent protein participates in lymphocyte activation. Cell, 61, 185-192 (1990)) found that PrP^{c} is expressed with similar surface abundance on all lymphocytes, they did not indicate in any way the possibility of identifying any of these lymphocytes as the sites of replication of the prion.

Bendheim et al. (Nearly ubiquitous tissue distribution of the scrapie agent precursor protein. Neurology, 42, 149-156 (1992)) went as far as even to question the key role of the LRS in the infection route. This article discloses that PrP^{c} is widespread in non-neuronal tissues, but it, too, fails to identify the sites of extraneuronal replication of PrP entirely.

Lasmézas et al. (Immune system-dependent and -independent replication of the scrapie agent. J. of Virology, 70, 1292-1295 (1996)) carried out investigations on the infection route in a SCID mouse model, and reached the conclusions that the primary route of infection involves the LRS and, in particular, the follicular dendritic cells, while the secondary route of infection appears to be a direct neural spread from the peritoneum. Thus, the conclusions of Lasmézas et al. taught away from the findings of the present inventors as to the actual infection route. The same holds for O'Rourke et al. (SCID mouse spleen does not support scrapie agent replication. J. of General Virology, 75, 1511-1514 (1994)): they, too, investigated the role of the LRS for the spread of PrP with the aid of a SCID mouse model and came to the conclusion that FDCs are the site of PrP^{Sc} replication.

Further, a study conducted by Büeler et al. (Normal development and behaviour of mice lacking the neuronal cell-surface PrP protein. Nature, 356, 577-582 (1992)) on mice devoid of functional Prn-P genes showed that the ablation of PrP^{C} did not appear to provide any detrimental effect. Thus, the only conclusion that could be drawn from the teachings of Büeler et al. is that ablation and/or repression of the Prn-P gene would be the only possible therapeutic approach. No specific mention or indication of other therapeutic approaches, possibly concerning B and/or T-cells, can found or derived from the teachings of Büeler et al. The same holds for Blättler et al. (PrP-expressing tissue required for transfer of scrapie infectivity from spleen to brain. Nature, 389, 69-73 (1997)), who also identifies genetic ablation or repression as the only possible therapeutic approach. In fact, the disclosure of Blättler et al. does not allow for any specific, identification of a subset of the lymphohaemopoietic stem cells as being responsible of supporting the replication of the infective agent.

Denis et al. (T cells in hypersensitivity pneumonitis: effects of in vivo depletion of T cells in a mouse model. American Journal of Respiratory Cell and Molecular Biology, 6, 2, 183-189 (1992)) investigated the role of T-cells in the context of lung fibrosis, i.e. in the context of a disease which elicits a “classical” immune response. The depletion of T-cells in this context was taken into consideration, but was not accompanied by any successful attempt to employ such depletion for therapeutic purposes. The findings of Denis et al. could not provide any helpful or encouraging data or notions for the research on prion diseases.

WO 89/12458 discloses techniques for stimulating the cellular immunity and assaying the activated T-cells in order to strengthen the immune defense. Given the specific nature of the priori diseases, wherein the defensive function of the immune system is completely ruled out due to the domestic expression of PrP^{c} of the cells involved, it is clear that any therapeutic approach as suggested in WO 89/12458 would be useless for prion diseases. Thus, this document discloses notions which cannot be applied to the diseases investigated by the present inventors.

The finding according to the present invention that the key roles in the prion infectivity are played primarily by B-cells and secondarily by T-cells leads to designing strategies for avoiding the spread of prion diseases where such strategies are based on the removal or absence of the carriers of infectivity. In this context, a study by Buttke et al. (Positive selection of mouse B and T lymphocytes and analysis of isolated populations by flow cytometry. J. of Immunological Methods, 58, 1-2, 193-207 (1983)) teaches the in vitro distinction between mouse B-cells and T-cells based on an antibody. However, in no way do Buttke et al. refer to or envisage the therapeutic application of such distinction techniques for the purpose of avoiding the spread of prion diseases by using products and tissues wherefrom the B-cells and/or T-cells had been eliminated. The same lack of intention or reference with respect to prion diseases is to be pointed out with regard to the findings of Bertolini et al. (A new “two step” procedure for 4.5 log depletion of T and B cells in allogenic transplantation and of neoplastic cells in autologous transplantation, Bone Marrow Transplantation, 19, 6, 615-619 (1996)): these researches also limited their findings and interest to the aspect of B-cell and T-cell depletion per se, based on immunoaffinity.

Kitamura et al. (A B-cell-deficient mouse by targeted disruption of the membrane exon of the immunoglobulin m chain gene. Nature, 350, 423-426 (1991)) presents the μmMt mouse carrying a selective immunodeficiency affecting B-cell development. This μmMt mouse has been developed merely for research purposes the researchers neither suggest nor envisage the possibility of applying such teachings concerning a B-cell impaired animal for the purposes of avoiding the spread of prion diseases by using the biological products and tissues of this type of animal. The same holds for the findings of Mombaerts et al. (Rag-1 deficient mice have no mature B and T lymphocytes. Cell, 68, 869-877 (1992)) and Shinkai et al. (Rag-2 deficient mice lack mature lymphocytes owing to the inability to initiate V (D) J rearrangement. Cell, 68, 855-867 (1992)), whose only concern was also that of genetically orchestrating specific immune defects in order to stimulate various maturation stages of immunocompetent cells. At no time did Mombaerts et al. or Shinkai et al. acknowledge that model animals or organisms carrying such immune defects could provide the material basis for a therapeutic strategy against prion disease spread.

The realization that the prion disease infectivity is based on the role played by the B-cells and, secondarily, by the T-cells, also leads to the design of highly specific assays for determining the presence of such infectivity carriers as well as any other related assays. On the contrary, the notions available in the pertinent field deriving from previous research never taught or suggested such specific assays.

Kimberlin et al. (Pathogenesis of mouse scrapie: dynamics of agent replication in spleen, spinal cord and brain after infection by different routes. J. of Comparative Pathology, 89, 4, 551-562 (1979)) only identified spleen-as a major extraneural replication site, and never indicated or suggested the relevance and specificity of B-cells and T-cells for assay purposes.

Similarly, Millson et al. (Early distribution of radioactive liposomes and scrapie infectivity in mouse tissues following administration by different routes. Veterinary Microbiology, 4, 2, 89-99 (1979)) disclosed the notion, now entirely overruled, of accumulation (without replication) of the infectivity in liver, and were thus far from the realization of an assay based solely on the actual carriers of infectivity. Further, Millson et al. themselves questioned the accuracy of their own data on scrapie infectivity in various tissues since it was not known how much of the scrapie agent taken up by different tissues would actually be infectious rather than remaining in a non-infectious form. This is a clearly rougher approach to the provision of infectivity assays than the one disclosed by the present inventors.

Diomede et al. (Activation effects of a prion protein fragment [PrP-(106-206)] on human leukocytes. Biochemical Journal, 320, 563-570 (1996)) investigated the role of PBLs in prion disease spread and presented no mention or allegation of the specific role played by B-cells and T-cells. Thus, in no way could Diomede et al. have envisaged specific assays based on the now identified role played by the B-cells and T-cells as carriers of infectivity.

Caughey et al. (Detection of prion protein mRNA in normal and scrapie-infected tissues and cell lines. J. of General Virology, 69, 711-716 (1988)) dealt with PrP expression in spleen. However, there is no conclusion as to a possible correlation between such PrP expression and the ability of the spleen to harbour the “scrapie agent”. Again, it is clear that Caughey et al. could not have derived from their findings an assay based on the realization of the precise role played by the B-cells and T-cells in prion infectivity.

This review of the teachings to be found in or derived from the background knowledge of the pertinent field shows limits and prejudices which render the findings of the present invention highly original and non-obvious, as these findings depart and differ on many occasions from the directions given by the prior researches and studies. Also, the many studies conducted in the field and the often rough and generic results achieved are clear indicators of the fact that while it was felt that a more sophisticated understanding of prion diseases was needed, it was also very difficult to achieve such understanding.

### FURTHER ASPECTS AND PREFERRED EMBODIMENTS OF THE INVENTION

Still further, according to the present invention, selective suppression of tse-infected (which are of course in turn infective) B-cells can be accomplished by treatment with an adequate amount of antibody to a tse-infected B-cell marker, like e.g. a surface marker. One should anticipate that examining unusual dispositions of B-cells or T-cells or of their progenitors and products may be important. Preferably, this antibody recognizes the infective B-cell and not the stem cell, thus allowing for a later repopulation of B-cells by the stem cell. Preferably, procedures well known in the art may help in the preparation of such antibodies. Accordingly, the use of such antibodies in a diagnostic assay and a medicament comprising such an antibody are a further aspect contemplated by the present invention.

Therefore, a further aspect of the invention relates to an antibody directed against tse-infected B-cells, characterized in that said antibody shows specificity to a tse-infected B-cell marker. Such an antibody may be obtained e.g. by immunization of suitable host animals with tse-infected B-cells.

A further aspect of the invention relates to the use of such an antibody directed to tse-infected B-cells in a diagnostic assay.

A further aspect of the invention relates to a medicament, comprising said antibody directed to tse-infected B-cells.

A further aspect of the present invention relates to a ligand capable of identification of tse-infected B-cells, characterized in that specific interaction between said ligand and said tse-infected B-cell is based on the infectivity of said B-cell.

A further aspect of the invention relates to the use of a ligand capable of identification of tse-infected B-cells in a method of analysis of said B-cell.

A preferred use of a ligand capable of identification of tse-infected B-cells is characterized in that said B-cell is intact.

A further aspect of the present invention relates to the use of a ligand capable of identification of tse-infected B-cells in histochemical analysis of whole B-cells mounted on microscope slides.

Still further, according to the present invention, selective suppression of tse-infected (which may be in turn infective, at least when administered i.e. to indicator hosts) T-cells can be accomplished by treatment with an adequate amount of antibody to a tse-infected T-cell marker, like e.g. a surface marker. Preferably, this antibody recognizes the tse-infected T-cell and not the stem cell, thus allowing for a later repopulation of T-cells by the stem cell. Preferably, procedures well known in the art may help in the preparation of such antibodies. Accordingly, the use of such antibodies in a diagnostic assay and a medicament comprising such an antibody are a further aspect contemplated by the present invention.

Therefore, a further aspect of the invention relates to an antibody directed against tse-infected T-cells, characterized in that said antibody shows specificity to a tse-infected T-cell marker. Such an antibody may be obtained e.g. by immunization of suitable host animals with tse-infected T-cells.

A further aspect of the invention relates to the use of such an antibody directed to tse-infected T-cells in a diagnostic assay.

A further aspect of the invention relates to a medicament, comprising said antibody directed to tse-infected T-cells.

A further aspect of the present invention relates to a ligand capable of identification of tse-infected T-cells, characterized in that specific interaction between said ligand and said tse-infected T-cell is based on the infectivity of said T-cell.

A further aspect of the invention relates to the use of a ligand capable of identification of tse-infected T-cells in a method of analysis of said T-cell.

A preferred use of a ligand capable of identification of tse-infected T-cells is characterized in that said T-cell is intact.

A further aspect of the present invention relates to the use of a ligand capable of identification of tse-infected T-cells in histochemical analysis of whole T-cells mounted on microscope slides.

A further aspect of the present invention is the provision of a medicament comprising B-cell depletants for the treatment of pathologies where the depletion of B-cells and more particularly of infected B-cells is therapeutically effective.

A further object of the present invention is the use of B-cell depletants for the manufacture of a medicament for the treatment of transmissible spongiform encephalopathy in infected humans or animals. A “B-cell depletant” as referred to in the present application is a reagent or a kit of reagents which upon administration either alone, together or sequentially leads to depletion of B-cells in the organism being treated. Any B-cell, depletant known in the art may be used to achieve the above stated object of the present invention. Suitable B-cell depletants comprise either immunologically active biomolecules like e.g. anti B-cell antibodies as well as immunosuppressively-active chemical compounds.

Anti B-cell antibodies are antibodies which recognize determinants (membrane molecules) which are highly specific for B-cells or for B-cell subsets (e.g. for lineages or maturational stages of B-cells). The skilled man is however aware that number and identity of such B-cell specific determinants may vary among different species. Thus, a determinant which is B-cell specific in one species may be a non-specific determinant in another species.

For example, according to a widely accepted approach, all of the antibodies that react with a particular membrane molecule are grouped together as a “cluster of differentiation” (CD). Each new antibody that recognizes a membrane molecule is analyzed to determine if it falls within a recognized CD designation; if it does not, it is given a new CD designation reflecting a new membrane molecule. Although the CD nomenclature was originally developed for human leukocyte membrane molecules, the homologous membrane molecules found in other species, such as mice, are commonly referred to by the same CD designations.

Importantly, the present invention takes advantage of the fact that for any conceivable host organism (e.g. of mouse, hamster, sheep, cattle or human origin) the B-cell specific determinants are either known or may be easily determined by methods known in the art, such that appropriate “matching” antibodies are available or may be tailored on demand by any known method.

Accordingly, it has to be emphasized that anti B-cell antibodies as encompassed by the present invention are to be understood as specifically recognizing the B-cells of the specific host undergoing therapy or assay or body fluid or, tissue purification. (Obviously, analogous general considerations apply to anti T-cell antibodies as referred to hereinafter.)

As a non-limiting example, anti-μm antibodies as described by R. S. Fujinami et al. in Journal of Virology, 69, 1995, pp. 5152-5155, the disclosure of which is hereby incorporated by reference, are preferred B-cell depletants according to the present invention. A further example for a B-cell depletant according to the present invention is the LR1 antibody as further described hereinafter. A further example for a B-cell depletant according to the present invention is B220 antibody as further described hereinafter. Also antibodies to malignant B-lymphocytes, useful for the treatment of B-lymphocyte lymphoma, are often cross-reactive with normal B-cells and also can be used for the purposes of the present invention. Examples of such antibodies exist in the literature. E.g. Epstein et al. describe the preparation of two such antibodies, termed Lym-1 and Lym-2, in Two new Monoclonal Antibodies Lym-1 and Lym-2, Reactive with Human B-Lymphocytes and Derived Tumors, with Immunodl agnostic and Immnuotherapeutlc Potential, Cancer Research, 47, 830-840 (1987). Since it is possible that in some, if not in many cases, the B-cell population may not all share identical surface markers, it may be necessary to utilize more than one antibody to effectively achieve the desired depletion of B-cells. The present invention envisions the utilization of as many antibodies as necessary to accomplish this goal.

Further preferred anti B-cell antibodies contemplated for use in the manufacture of a medicament for the treatment or prevention of transmissible spongiform encephalopathy in infected humans or animals are chimaeric anti B-cell antibodies. The manufacture of chimaeric antibodies is described e.g. in U.S. Pat. No. 5,681,722, which is hereby incorporated by reference. Chimaeric antibodies entail the advantage that they can be designed so as not to be immunogenic to the host organism undergoing treatment. Thus, such specifically designed chimaeric antibodies do not induce the treated host organism's anti antibody response. As is true for any other antibodies contemplated by the invention, such chimaeric antibodies can be used either in their native form or as part of an antibody/chelate, antibody/drug or antibody/toxin complex.

Thus, a specifically preferred anti B-cell antibody contemplated for use in the manufacture of a medicament for the treatment or prevention of transmissible spongiform encephalopathy in infected humans is rituximab (also known as C2B8 or rituxin), a chimaeric mouse-human antibody which binds to- and rapidly depletes the human immune system's B-cells but leaves stem cells, pre-B-cells, dendritic cells, T-cells, NK cells and plasma cells unaffected.

Further, as a general aspect, the present invention envisions the use of unmodified (i.e. ‘naked’) antibodies as well as of antibodies conjugated with a suitable cytotoxic agent, toxin or radionuclide. Appropriate radioisotopes include ¹³¹I, ⁹⁰Y, ⁶⁷Cu. Procedures for the preparation of iodinated antibodies are well-known in the art and such preparations can be carried out easily in hospital radiopharmacies.

The antibody also can be conjugated, by procedures described in the art with known cytotoxic drugs such as methotrexate, aminopterin, mitoxantrone, vincristine, vinblastine, doxorubicin and others, or with plant toxins such as abrin, or ricin or the like or their ribosome-inactivating sub-units, or any other agents known to have cytotoxic properties.

In addition, the present invention contemplates the use of genetically, enzymatically, or chemically altered antibodies which recognize B-cells, whereby the constant regions have been altered or replaced with domains which fix complement proteins or elicit target cell destruction by virtue of antibody-dependent cellular cytotoxicity (ADCC), thus activating the patient's own immune system.

Further non-limiting examples of B-cell depletants contemplated by the present invention are chemical compounds like ciamexone, i.e. 2-cyano-1-[(2-methoxy-6-methylpyridin-3-yl)-methyl]-aziridine (U.S. Pat. No. 5,055,290) and imexon, i.e. 4-imino-1,3-diazabicyclo-(3.1.0)-hexan-2-one (U.S. Pat. No. 5,369,119) the disclosures of which are hereby incorporated by reference. Imexon is known to act specifically on B-cells in that it suppresses B-cell proliferation or B-cell activation. On the other hand, ciamexone seems to suppress B-cell proliferation caused by B-cell growth factor; hence, it may be said that ciamexone suppresses BCGF-induced B-cell proliferation.

As a general aspect, the therapeutic compositions (i.e. the medicaments) of the present invention can be administered parenterally by injection, rapid infusion, nasopharyngeal absorption (intranasopharangally), dermoabsorption, orally, intraocularly, or intracerebroventricularly (i.c.v.). The compositions may alternatively be administered intramuscularly, or intravenously. Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers or occlusive dressings can be used to increase bioavailability. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring or perfuming agents.

According to the present invention, an “effective amount” of the medicament is one which is sufficient to achieve the desired biological effect. Generally, the dosage needed to provide an effective amount of the medicament will vary depending upon such factors as the human's or animal's age, condition, sex, and extent of disease, if any, and other variables which can be adjusted by one of ordinary skill in the art.

A further object of the present invention is the provision of a product comprising cyclophosphamide and dexamethasone as a combined preparation for the simultaneous, separate or sequential use in the treatment or prevention of transmissible spongiform encephalopathy in infected humans or animals. Still a further object of the invention is the use of a combination of cyclophosphamide and dexamethasone either in a combined dosage form or in separate dosage forms for the manufacture of a medicament for the treatment or prevention of transmissible spongiform encephalopathy in infected humans or animals.

Above aspects of the present invention are based on the fact that the present inventors have surprisingly found out that a combined treatment with cyclophosphamide and dexamethasone, by virtue of the simultaneous B-cell and T-cell depletion achieved thereby, leads to heretofore unachieved therapeutic results.

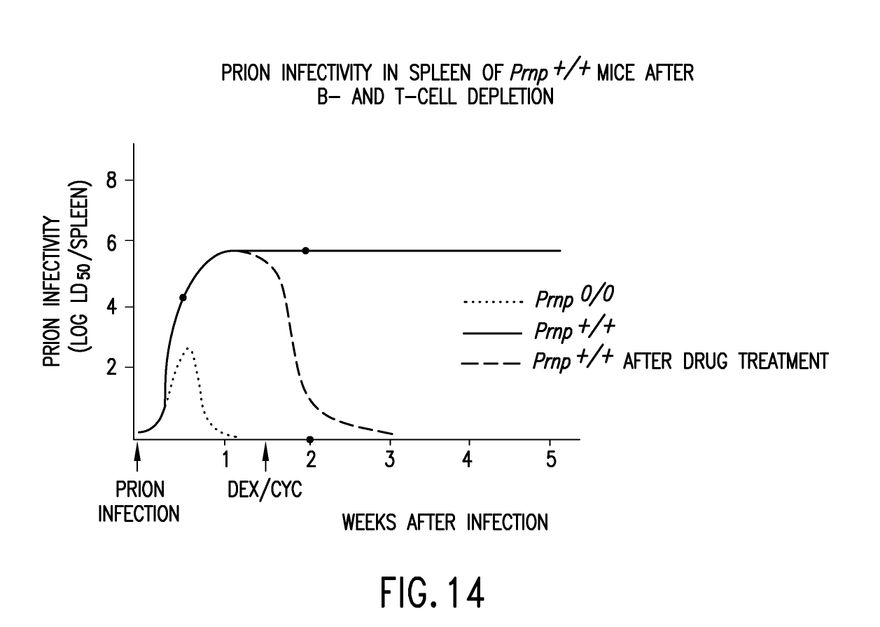

Importantly, it has been shown by the inventors that above combination treatment, even if triggered as late as 10 days after i.p. inoculation, leads to total clearance of infectivity from the spleens of i.p. infected test animals. This finding is particularly important in that it is known from the literature (see e.g. Büeler, H. R. et al. Mice devoid of PrP are resistant to scrapie. Cell 73, 1339-1347 (1993)) that establishment of a plateau of the infectious titer in the spleen normally takes place at a relatively early time point, i.e. after about 1 week or even less (see FIG. 14). Thus, such combination treatment proposed by the present inventors is fully effective even after—or very close to achievement of the maximum of infectivity in spleen, i.e. presumably during the whole incubation period characterized by constant spleenic infectivity and before such infectivity becomes detectable in brain. The skilled reader will readily appreciate that such a therapeutic approach giving successful results (i.e. not only prolongation of the incubation period but importantly complete absence of measurable infectivity) after inoculation and more importantly after achievement of the plateau level provides for a unique breakthrough. Indeed, it is only the post-inoculation potency which renders any therapeutic approach feasible for the treatment of e.g. presumably infected human individuals. In contrast thereto, pre or co-inoculation potencies as reported in older literature would only be of academic value since they would require the patient's exact knowledge about the time point of inoculation which is obviously impossible as e.g. in case of oral intake.

A further object of the present invention is the provision of a diagnostic method allowing the determination of the presence or absence of tse-infected B-cells in humans or animals or in body fluid or tissue derived products isolated therefrom. Such assay method comprises the steps of extracting B-cells from body fluids or from tissue or from products derived therefrom and inoculating said B-cells into the cerebrum of a test animal, development of transmissible spongiform encephalopathy in said test animal indicating presence of said tse-infected B-cells.

As to the extraction step, the invention contemplates any method known in the art suitable for selective extraction, of B-cells or of their progenitors or products from a body fluid or tissue sample drawn from the human or animal undergoing diagnosis. As will be apparent to the man skilled in the art, such extraction involves use of a reactive physical entity specifically recognizing B-cells, preferably B-cell specific antibodies, as the ones described herein. Thus, the extraction will preferably be analogous to the separation methods adopted for the manufacture of non-infective body fluid or tissue derived products which are detailed later. In a preferred but not limiting embodiment of the present invention, the inventors have used anti-mouse-B220 antibodies conjugated with super-paramagnetic microbeads (Milteny Biotec GmbH, Germany) for the purification of B-cells.

Suitable test animals for carrying out the method of the present invention are e.g. tga20 indicator mice and as they were used by Brandner et al. in ‘Normal host prion protein necessary for scrapie-induced neurotoxicity’, Nature, 379, (1996), the disclosure of which is hereby incorporated by reference. As reported by Brandner, infectivity of a given inoculum determines the incubation time elapsed before the appearance of clinical symptoms displayed by the test animals. (see Table 5) Accordingly, the use of purified fractions containing high titers of B-cells constitutes an advantage provided by the assay methods of the present invention.

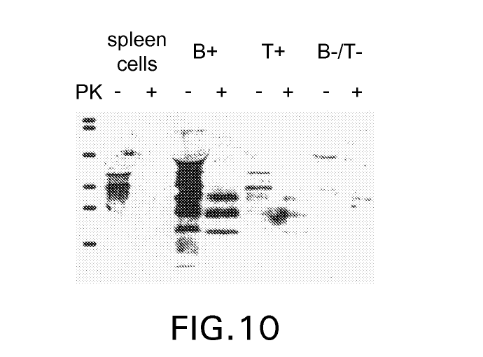

Further to the improved bioassay discussed above, the present invention also provides an assay method for determination of the presence of tse-infected B-cells in humans or animals or in body fluid or tissue derived products isolated therefrom, characterized in that the B-cells are subjected-to a Western blot analysis with an anti-PrP antibody either directly and after having been digested with proteinase K. Also this aspect of the present invention is based on the finding that identification of the crucial carrier of PrP^{Sc} allows for the design of more sensitive assays. An example is apparent from FIG. 10, showing that purification of the B-cells prior to carrying out the Western blot with mab 6H4 leads to enrichment of PrP^{Sc}. Of course, as an obvious equivalent of mab 6H4, any other anti-PrP antibody could be used.

A further object of the invention is the provision of a non tse-infective body fluid product. Thus, according to the invention, a non tse-infective body fluid product is a body fluid product which is substantially free of B-cells. A preferred body fluid product according to the invention is a blood product, like e.g. plasma (or fractions thereof, like Cohn fractions) or buffy coat which is totally purified from B-cell and/or from B-cell debris. Further aspects of the present invention relate to the use of B-cell depleted body fluid or tissue derived products for the prevention of transmissible spongiform encephalopathy spread in human or animal populations. In particular, the use of body fluid or tissue derived, but still cells or cellular debris containing products is encompassed by the invention. As shown hereinabove, the B-cells play a crucial role in the spread of infectivity. Thus, the B-cells, which have been identified here as the primary carriers of tse-infectivity and preferably also T-cells (which in i.p. tse-infected organisms, are likely to undergo rapid secondary infection) should be completely removed in order to establish the safety of biological material derived for e.g. transplantation or transfusion purposes from human or animal sources. Therefore, known purification protocols for the manufacture of such body fluid or tissue derived products, especially if they contain still whole cells (like e.g. buffy coat) or cellular debris (like crude plasma), should be redesigned so as to comprise a specific B-cell depletion (and preferably also a T-cell depletion) step. In the case of cellular debris containing products, it is particularly preferred that B-cell (and preferably also T-cell) depletion is carried out before such cellular debris is formed. That is to say, adequate precursors of cellular debris containing products should be B- (and preferably T-) cell depleted. Thus, a body fluid or tissue derived product so obtained would be a non tse-infective body fluid or tissue derived product.

As a non limiting example for a non tse-infective body fluid derived product, the present invention provides buffy coat, characterized in that it has been depleted of B-cells in vitro.

As outlined above, a further aspect of the invention is the provision of a non-infective tissue derived product. Thus, according to the invention, a non-infective tissue derived product is a tissue derived product which is substantially free of B-cells. A preferred tissue derived product according to the invention is a product derived from the lymphoreticular system. A still preferred tissue derived product according to the invention is a spleen derived product.

A further aspect of the invention contemplated above, is a method of manufacture of a non-infective body fluid product. Thus, according to the invention, non-infective body fluid products are obtained by specifically separating B-cells from body fluids or from known body fluid products. Though any suitable method known to the man skilled in the art could be used for the specific separation of B-cells from body fluids, specific separation by means of B-cell specific immunoreactants like e.g. B-cell specific antibodies is preferred. Suitable but not limiting examples of such B-cell specific antibodies are commercially available B220 or LR1 antibodies or anti-μm antibodies, vide supra. The term “specific separation by means of B-cell specific antibodies” encompasses any separation method which comprises the use of separation reagents comprising B-cell specific antibodies for the recognition of B-cells in body fluid products. Separation reagents comprising B-cell specific antibodies are B-cell specific antibodies which are conjugated to a solid phase or which are capable of interacting with a solid phase via chemical or physical means either by themselves or by virtue of suitable derivatization in such a manner that they get either directly or indirectly immobilized on said solid phase so as to enable separation from the reaction mixture.

In particular, the present invention provides a method for the provision of buffy coat, characterized in that such buffy coat is contacted with anti B-cell antibodies linked to a solid support.

Further, the present invention provides a method for the purification of plasma, characterized in that such plasma or a precursor used in the preparation thereof is contacted with anti B-cell antibodies linked to a solid support.

A further aspect of the invention contemplated above is a method of manufacture of such a non-infective tissue derived product. Thus, according to the invention, non-infective tissue derived products are obtained by specifically separating B-cells from tissue derived products. Though any suitable method known to the man skilled in the art could be used for the specific separation of B-cells from tissue derived products, specific separation by means of B-cell specific immunoreactants like e.g. B-cell specific antibodies is preferred. Suitable but not limiting examples of such B-cell specific antibodies are commercially available B220 or LR1 antibodies or anti-μm antibodies. The term “specific separation by means of B-cell specific antibodies” encompasses any separation method which comprises the use of separation reagents comprising B-cell specific antibodies for the recognition of B-cells in tissue derived products. Separation reagents comprising B-cell specific antibodies are B-cell specific antibodies which are conjugated to a solid phase or which are capable of interacting with a solid phase via chemical or physical means either by themselves or by virtue of suitable derivatization in such a manner that they get either directly or indirectly immobilized on said solid phase so as to enable separation from the reaction mixture.

Still further, according to the invention, non-infective body fluid products and/or tissue derived products are obtained from B-cell depleted organisms. Any method known to the man skilled in the art can be used for the depletion of B-cells in organisms. For example, organisms can “be treated with anti-μm antibodies as described by R. S. Fujinami et al. vide supra, so as to become sources of B-cell depleted peripheral blood. A further method for the depletion of B-cells in organisms may be selective knock out of B-cell related genes. A suitable but non-limiting example of an organism obtained by knocking out B-cell related genes is the μMT mouse described by Kitamura et al. ‘A B-cell deficient mouse by targeted disruption of the membrane exon of the immunoglobulin mu-chain gene’ Nature 350, 423-426 (1991), the disclosure of which is hereby incorporated by reference. Thus, according to the invention, μMT mice are a suitable source for B-cell depleted blood products and/or tissue derived products.

Thus, a further aspect of the invention is a method for the manufacture of plasma or buffy coat, characterized in that plasma or buffy coat are isolated from B-cell deficient animals. In this context, a preferred method would encompass the generation of B-cell deficient animals by removing or inhibiting expression of B-cell related genes contained therein.

A further aspect of the present invention relates to the B-cell mediated secondary tse-infection of T-cells. As described above, such secondary tse-infection of the T-cells is not an alternative route of invasion of an infected human's or animal's LRS, but it is instead strictly depending on a previous tse-infection taken up by the B-cells. Therefore, depending on the progress of disease, measures directed to the coping with the presence of such tse-infected T-cells are a further aspect of the present invention.

In view of the above, the present invention provides a medicament comprising T-cell depletants, for the treatment of pathologies where the depletion of T-cells, and more particularly of tse infected T-cells is therapeutically effective.

According to a further aspect of the invention, the use of T-cell depletants for the manufacture of a medicament for the treatment or prevention of transmissible spongiform encephalopathy in infected humans or animals is provided. A “T-cell depletant” as referred to in the present application is a reagent or a kit of reagents which upon administration either alone, together or sequentially leads to depletion of T-cells in the organism being treated. Any T-cell depletant known in the, art may be used to achieve the above stated object of the present invention. Suitable T-cell depletants comprise either immunologically active biomolecules like e.g. anti T-cell antibodies as well as immunosuppressively-active chemical compounds.

Anti T-cell antibodies are antibodies which recognize determinants (membrane molecules) which are highly specific for T-cells or for T-cell subsets (e.g. for lineages or maturational stages of T-cells). The skilled man is however aware that number and identity of such T-cell specific determinants may vary among different species. Thus, a determinant which is T-cell specific in one species may be a non-specific determinant in another species.

For example, according to a widely accepted approach, all of the antibodies that react with a particular membrane molecule are grouped together as a “cluster of differentiation” (CD). Each new antibody that recognizes a membrane molecule is analyzed to determine if it falls within a recognized CD designation; if it does not, it is given a new CD designation reflecting a new membrane molecule. Although the CD nomenclature was originally developed for human leukocyte membrane molecules, the homologous membrane molecules found in other species, such as mice, are commonly referred to by the same CD designations.

Importantly, the present invention takes advantage of the fact that for any conceivable host organism (e.g. of mouse, hamster, sheep, cattle or human origin) the T-cell specific determinants are either known or may be easily determined by methods known in the art, such that appropriate “matching” antibodies are available or may be tailored on demand by any known method.

Accordingly, it has to be emphasized that anti T-cell antibodies as encompassed by the present invention are to be understood as specifically recognizing the T-cells of the specific host undergoing therapy or assay or body fluid or tissue purification.

A non-limiting example for a suitable anti T-cell antibody acting as T-cell depletant is the Thy1.2 antibody as described hereinafter. A further non-limiting example for a T-cell depletant cyclic peptide is Cyclosporin A, as it is described e.g. in Römpp Lexikon Biotechnologie, 1992, Thieme Verlag, Stuttgart, Germany.

A further object of the present invention is the provision of a diagnostic method allowing the determination of the presence or absence of infective T-cells in humans or animals or in body fluid or tissue derived products isolated therefrom. Such assay method comprises the steps of extracting T-cells from body fluids or from tissue or from products derived therefrom and inoculating said T-cells into the cerebrum of a test animal, development of transmissible spongiform encephalopathy in said test animal indicating presence of said infective T-cells.

As to the extraction step, the invention contemplates any method known in the art suitable for selective extraction of T-cells or of their progenitors or products from a body fluid or tissue sample drawn from the human or animal undergoing diagnosis. As will be apparent to the man skilled in the art, such extraction involves use of a reactive physical entity specifically recognizing T-cells, preferably anti T-cell specific antibodies, as the ones described hereinbelow. Thus, the extraction will preferably be analogous to the separation methods adopted for the manufacture of non-infective body fluid or tissue derived products which, are detailed later. In a preferred but not limiting embodiment of the present invention, the inventors have used anti-mouse-Thy1.2 antibodies conjugated with super-paramagnetic microbeads (Milteny Biotec GmbH, Germany) for the purification of T-cells.

Suitable test animals for a bioassay as above are tga 20 indicator mice or others known in the art.

Further to the improved bioassay discussed above, the present Invention also provides an assay method for determination of the presence of tse-infected T-cells in humans or animals or in body fluid or tissue derived products isolated therefrom, characterized in that the T-cells are subjected to a Western blot analysis with an anti-PrP antibody either directly fend, after having been digested with proteinase K. Also this aspect of the present invention is based on the finding that identification of specific cell types infected with PrP^{Sc} allows for the design of more sensitive assays. An example is apparent from FIG. 10 showing that purification of the T-cells prior to carrying out the Western blot analysis improves the results.

A further object of the invention is the provision of a non-infective body fluid product. Thus, according to the invention, a non-infective body fluid product is a body fluid product which is substantially free of T-cells. A preferred body fluid product according to the invention is a blood product, like e.g. plasma (or fractions thereof, like Cohn fractions) or buffy coat which is totally purified from T-cell and/or from T-cell debris. Further aspects of the present invention relate to the use of T-cell depleted body fluid or tissue derived products for the prevention of transmissible spongiform encephalopathy spread in human or animal populations. In particular the use of non tse-infective, body fluid or tissue derived, but still cells or cellular debris containing products is encompassed by the invention. Therefore, the invention provides buffy coat, characterized in that it has been depleted from T-cells in vitro.

A further aspect of the invention is the provision of a non-infective tissue derived product. Thus, according to the invention, a non-infective tissue derived product is a tissue derived product which is substantially free of T-cells. A preferred tissue derived product according to the invention is a product derived from the lymphoreticular system. A still preferred tissue derived product according to the invention is a spleen derived product.

A further aspect of the invention is a method of manufacture of a non-infective body fluid product. Thus, according to the invention, non-infective body fluid products are obtained by specifically separating T-cells from body fluids or from known body fluid products. Though any suitable method known to the man skilled in the art could be used for the specific separation of T-cells from body fluids, specific separation by means of T-cell specific immunoreactants like e.g. T-cell specific antibodies is preferred. A suitable but not limiting example of such a T-cell specific antibody is Thy1.2. The term “specific separation by means of T-cell specific antibodies” encompasses any separation method which comprises the use of separation reagents comprising T-cell specific antibodies for the recognition of T-cells in body fluid products. Separation reagents comprising T-cell specific antibodies are T-cell specific antibodies which are conjugated to a solid phase or which are capable of interacting with a solid phase via chemical or physical means either by themselves or by virtue of suitable derivatization in such a manner that they get either directly or indirectly immobilized on said solid phase so as to enable separation from the reaction mixture. In particular, the present invention provides a method for the provision of buffy coat, characterized in that such buffy coat is contacted with anti T-cell antibodies linked to a solid support. Still further, the present invention provides a method for the purification of plasma characterized in that such plasma or a precursor used in the preparation thereof is contacted with anti T-cell antibodies linked to a solid support.

A further aspect of the invention contemplated above is a method of manufacture of such a non-infective tissue derived product. Thus, according to the invention, non-infective tissue derived products are obtained by specifically separating T-cells from tissue derived products. Though any suitable method known to the man skilled in the art could be used for the specific separation of T-cells from tissue derived products, specific separation by means of T-cell specific immunoreactants like e.g. T-cell specific antibodies is preferred. A suitable but not limiting example of such a T-cell specific antibody is Thy1.2. The term “specific separation by means of T-cell specific antibodies” encompasses any separation method which comprises the use of separation reagents comprising T-cell specific antibodies for the recognition of T-cells in tissue derived products. Separation reagents comprising T-cell specific antibodies are T-cell specific antibodies which are conjugated to a solid phase or which are capable of interacting with a solid phase via chemical or physical means either by themselves or by virtue of suitable derivatization in such a manner that they get either directly or indirectly immobilized on said solid phase so as to enable separation from the reaction mixture.

As pointed out above, the present invention provides further an assay method for monitoring the progress of transmissible spongiform encephalopathy. Said assay method comprises the extraction of B-cells and T-cells from body fluid or tissue samples drawn from the human or animal undergoing diagnosis. Extraction of both physical entities can be carried out either simultaneously or sequentially. The purified B and T-cell fractions thus obtained may be further purified by complement lysis of B-cells in the T-cell fraction and vice versa. Suitable but non-limiting examples for antibodies suitable complement lysis in vitro are rat anti mouse LR1 antibody (clone LR6.2B6D6.C9, Serotec) and mouse anti mouse antibody Thy1.2 (clone F7D5, Serotec). Of course, such an assay method may be also easily modified for the monitoring of transmissible encephalopathy therapy.

Obviously as in the case of the assay aimed at monitoring the disease progress, all the above (and further) aspects of the invention are not to be considered as being mutually exclusive, as far as B and T-cells are concerned. Therefore, according to the disease progress, the T-cell related measures according to the invention may be carried out simultaneously, consecutively or in a concerted manner with the B-cell related measures contemplated by the present invention.

Further details are set out in the description of the methods contemplated by the present invention and in the examples.

### Methods Contemplated by the Present Invention

### Immunoassays and Generation of Ligands Capable of Identification of Tse-Infected B-Cells or T-Cells

B-cells, and more particularly tse-infected B-cells shown above to be capable of transmitting spongiform encephalopathy are important for the generation of specific immunological reagents, antigens and antibodies which can be utilized in a variety of assays, many of which are described herein, for the detection of transmissible spongiform encephalopathy (TSE). They can be used as immunogens to produce antibodies. These antibodies can be, for example, polyclonal or monoclonal antibodies, chimeric, single chain and humanized antibodies, as well as Fab fragments, or the product of an Fab expression library. Various procedures known in the art may be used for the production of such antibodies and fragments.

For example, antibodies generated against a preparation of tse-infected B-cells can be obtained, by direct injection of the tse-infected B-cells into an animal. A mouse, rabbit or goat is preferred. The antibody so obtained then will bind the tse-infected B-cells, that is to say such antibody is specific to a tse-infected B-cell marker, like e.g. a surface marker thereof. Such antibodies then can be used to isolate the tse-infected B-cells from test samples as tissue suspected of containing infectious material. For preparation of monoclonal antibodies, any technique which provides antibodies produced by continuous cell line cultures can be used. Examples include the hybridoma technique as described by Kohler and Milstein, Nature 256:495-497 (1975), the trioma technique, the human B-cell hybridoma technique as described by Kozbor et al, Immun. Today 4:72 (1983) and the EBV-hybridoma technique to produce human monoclonal antibodies as described by Cole et al., in Monoclonal Antibodies and Cancer Therapy, Alan R. Liss, Inc, New York, N.Y., pp. 77-96 (1985). Techniques described for the production of single chair antibodies can be adapted to produce single chain antibodies to immunogenic polypeptide products of this invention. See, for example, U.S. Pat. No. 4,946,778, which is incorporated herein by reference.

Various assay formats may utilize the antibodies of the present invention, including “sandwich” immunoassays and probe assays. For example, the antibodies of the present invention, or fragments thereof, can be employed in various assays to determine the presence, if any, of tse-infected B-cells in a test sample. For example, in a first assay format, a polyclonal or monoclonal antibody or fragment thereof, or a combination of these antibodies, which has been coated’ on a solid phase, is contacted with a test sample, to form a first mixture. This first mixture is incubated for a time and under conditions sufficient to form antigen/antibody complexes. Then, an indicator reagent comprising a monoclonal or a polyclonal antibody or a fragment thereof, or a combination of these antibodies, to which a signal generating compound has been attached, is contacted with the antigen/antibody complexes to form a second mixture. This second mixture then is incubated for a time and under conditions sufficient to form antibody/antigen/antibody complexes. The presence of tse-infected B-cells in the test sample and captured on the solid phase, if any, is determined by detecting the measurable signal generated by the signal generating compound. The amount of tse-infected B-cell antigen present in the test sample is proportional to the signal generated.

In an alternative assay format, a mixture is formed by contacting: (1) a polyclonal antibody, monoclonal antibody, or fragment thereof, which specifically binds to tse-infected B-cells, or a combination of such antibodies bound to a solid support; (2) the test sample; and (3) an indicator reagent comprising a monoclonal antibody, polyclonal antibody, or fragment thereof, which specifically binds to a different tse-infected B-cell antigen (or a combination of these antibodies) to which a signal generating compound is attached. This mixture is incubated for a time and under conditions sufficient to form antibody/antigen/antibody complexes. The presence, if any, of tse-infected B-cell antigen present in the test sample and captured on the solid phase is determined by detecting the measurable signal generated by the signal generating compound. The amount of tse-infected B-cell antigen present in the test sample is proportional to the signal generated.

In another assay format, one or a combination of at least two monoclonal antibodies of the invention can be employed as a competitive probe for the detection of antibodies to tse-infected B-cell antigen. For example, infective B-cells can be gently lysed and coated on a solid phase. A test sample suspected of containing antibody to tse-infected B-cell antigen then is incubated with an indicator reagent comprising a signal generating compound and at least one monoclonal antibody of the invention for a time and under conditions sufficient to form antigen/antibody complexes of either the test sample and indicator reagent bound to the solid phase or the indicator reagent bound to the solid phase. The reduction in binding of, the monoclonal antibody to the solid phase can be quantitatively measured.

In yet another detection method, each of the monoclonal or polyclonal antibody of the present invention can be employed in the detection of tse-infected B-cell antigens in tissue sections, as well as in cells, by immunohistochemical analysis. Cytochemical analysis wherein these antibodies are labeled directly (with, for example, fluorescein, colloidal gold, horseradish peroxidase, alkaline phosphatase, etc.) or are labeled by using secondary labeled anti-species antibodies (with various labels as exemplified herein) to track the histopathology of disease also are within the scope of the present invention.

In addition, these monoclonal antibodies can be bound to matrices similar to CNBr-activated Sepharose and used for the affinity purification of specific tse-infected B-cells or tse-infected B-cell antigens from cell cultures or biological tissues such as to purify recombinant and native tse-infected B-cell proteins or to prepare biological tissue or fluid devoid of tse-infected B-cells.

The monoclonal antibodies of the invention also can be used for the generation of chimeric antibodies for therapeutic use, or other similar applications.

The monoclonal antibodies or fragments thereof can be provided individually to detect tse-infected B-cells. Combinations of the monoclonal antibodies (and fragments thereof) provided herein also may be used together as components in a mixture or ‘cocktail’ of at least one tse-infected B-cell antibody of the invention, along with antibodies which specifically bind to other tse-infected B-cell regions, each antibody having different binding specificities. Thus, this cocktail can include the monoclonal antibodies of the invention which are directed to tse-infected B-cell polypeptides and other monoclonal antibodies specific to other antigenic determinants of tse-infected B-cells.

The polyclonal antibody or fragment thereof which can be used in the assay formats should specifically bind to a tse-infected B-cell polypeptide or other tse-infected B-cell polypeptides additionally used in the assay. The polyclonal antibody used preferably is of mammalian origin such as, human, goat, rabbit or sheep polyclonal antibody which binds tse-infected B-cells. Most preferably, the polyclonal antibody is of rabbit origin. The polyclonal antibodies used in the assays can be used either alone or as a cocktail of polyclonal antibodies. Since the cocktails used in the assay formats are comprised of either monoclonal antibodies or polyclonal antibodies having different binding specificity to tse-infected B-cells, they are useful for the detecting, diagnosing, staging, monitoring, prognosticating, preventing or treating, or determining the predisposition to transmissible spongiform encephalopathy.

It is contemplated and within the scope of the present invention that tse-infected B-cells or specific antigens thereof may be detectable in assays by use of a recombinant antigen as well as by use of a synthetic peptide or purified peptide, which peptide comprises an amino acid sequence of tse-infected B-cells. It also is within the scope of the present invention that different synthetic, recombinant or purified peptides, identifying different epitopes of tse-infected B-cells, can be used in combination in an assay for the detecting, diagnosing, staging, monitoring, prognosticating, preventing or treating, or determining the predisposition to transmissible spongiform encephalopathy. In this case, all of these peptides can be coated onto one solid phase; or each separate peptide may be coated onto separate solid phases, such as’ microparticles, and then combined to form a mixture of peptides which can be later used in assays. Furthermore, it is contemplated that multiple peptides which define epitopes from different antigens may be used for the detection, diagnosis, staging, monitoring, prognosis, prevention or treatment of, or determining the predisposition to transmissible spongiform encephalopathy. Peptides coated on solid phases or labeled with detectable labels are then allowed to compete with those present in a patient sample (if any) for a limited amount of antibody. A reduction in binding of the synthetic, recombinant, or purified peptides to the antibody (or antibodies) is an indication of the presence of tse-infected B-cells antigen in the patient sample. The presence of tse-infected B-cells antigen indicates the presence of transmissible spongiform encephalopathy in the patient. Variations of assay formats are known to those of ordinary skill in the art and many are discussed herein below.

In another assay format, the presence of anti tse-infected B-cell antibody and/or tse-infected B-cell antigen can be detected in a simultaneous assay, as follows. A test sample is simultaneously contacted with a capture reagent of a first analyte, wherein said capture reagent comprises a first binding member specific for a first analyte attached to a solid phase and a capture reagent for a second analyte, wherein said capture reagent comprises a first binding member for a second analyte attached to a second solid phase, to thereby form a mixture. This mixture is incubated for a time and under conditions sufficient to form capture reagent/first analyte and capture reagent/second analyte complexes. These so-formed complexes then are contacted with an indicator reagent comprising a member of a binding pair specific for the first analyte labeled with a signal generating compound and an indicator reagent comprising a member of a binding pair specific for the second analyte labeled with a signal generating compound to form a second mixture. This second mixture is incubated for a time and under conditions sufficient to form capture reagent/first analyte/indicator reagent complexes and capture reagent/second analyte/indicator reagent complexes. The presence of one or more analytes is determined by detecting a signal generated in connection with the complexes formed on either or both solid phases as an indication of the presence of one or more analytes in the test sample. In this assay format, recombinant antigens derived from the expression systems disclosed herein may be utilized, as well as monoclonal antibodies produced from the proteins derived from the expression systems as disclosed herein. For example, in this assay system, infective B-cell antigen can be the first analyte. Such assay systems are described in greater, detail in EP Publication No. 0473065.

In yet other assay formats, the polypeptides disclosed herein may be utilized to detect the presence of antibody against tse-infected B-cell antigen in test samples. For example, a test sample is incubated with a solid phase to which at least one polypeptide such as a recombinant protein or synthetic peptide has been attached. These are reacted for a time and under conditions sufficient to form antigen/antibody complexes. Following incubation, the antigen/antibody complex is detected. Indicator reagents may be used to facilitate detection, depending upon the assay, system chosen. In another assay format, a test sample is contacted with a solid phase to which a recombinant protein produced as described herein is attached, and also is contacted with a monoclonal or polyclonal antibody specific for the protein, which preferably has been labeled with an indicator reagent. After incubation for a time and under conditions sufficient for antibody/antigen complexes to form, the solid phase is separated from the free phase, and the label is detected in either the solid or free phase as an indication of the presence of antibody against tse-infected B-cell antigen. Other assay formats utilizing the recombinant antigens disclosed herein are contemplated. These include contacting a test sample with a solid phase to which at least one antigen from a first source has been attached, incubating the solid phase and test sample for a time and under conditions sufficient to form antigen/antibody complexes, and then contacting the solid phase with a labeled antigen, which antigen is derived from a second source different from the first source. For example, a recombinant protein derived from a first source such as E. coli is used as a capture antigen on a solid phase, a test sample is added to the so-prepared solid phase, and following standard incubation and washing steps as deemed or required, a recombinant protein derived from a different source (i.e., non-E. coli) is utilized as a part of an indicator reagent which subsequently is detected. Likewise, combinations of a recombinant antigen on a solid phase and synthetic peptide in the indicator phase also are possible. Any assay format which utilizes an antigen specific for tse-infected B-cells produced or derived from a first source as the capture antigen and an antigen specific for tse-infected B-cells from a different second source is contemplated. Thus, various combinations of recombinant antigens, as well as the use of synthetic peptides, purified proteins and the like, are within the scope of this invention. Assays such as this and others are described in U.S. Pat. No. 5,254,458, which enjoys common ownership and is incorporated herein by reference.

Other embodiments which utilize various other solid phases also are contemplated and are within the scope of this invention. For example, ion capture procedures for immobilizing an immobilizable reaction complex with a. negatively charged polymer (described in EP publication 0326100 and EP publication No. 0406473), can be employed according to the present invention to effect a fast solution-phase immunochemical reaction. An immobilizable immune complex is separated from the rest of the reaction mixture by ionic interactions between the negatively charged poly-anion/immune complex and the previously treated, positively charged porous matrix and detected by using various signal generating systems previously described, including those described in chemiluminescent signal measurements as described in EPO Publication No. 0 273,115.

Also, the methods of the present invention can be adapted for use in systems which utilize microparticle technology including automated and semi-automated systems wherein the solid phase comprises a microparticle (magnetic or non-magnetic). Such systems include those described in, for example, published EPO applications Nos. EP 0 425 633 and EP 0 424 634, respectively.

The use of scanning probe microscopy (SPM) for immunoassays also is a technology to which the monoclonal antibodies of the present invention are easily adaptable. In scanning probe microscopy, particularly in atomic force microscopy, the capture phase, for example, at least one of the monoclonal antibodies of the invention, is adhered to a solid phase and a scanning probe microscope is utilized to detect antigen/antibody complexes which may be present on the surface of the solid phase. The use of scanning tunneling microscopy eliminates the need for labels which normally must be utilized in many immunoassay systems to detect antigen/antibody complexes. The use of SPM to monitor specific binding reactions can occur in many ways. In one embodiment, one member of a specific binding partner (analyte specific substance which is the monoclonal antibody of the invention) is attached to a surface suitable for scanning. The attachment of the analyte specific substance may be by adsorption to a test piece which comprises a solid phase of a plastic or metal surface, following methods known to those of ordinary skill in the art. Or, covalent attachment of a specific binding partner (analyte specific substance) to a test piece which test piece comprises a solid phase of derivatized plastic, metal, silicon, or glass may be utilized. Covalent attachment methods are known to those skilled in the art and include a variety of means to irreversibly link specific binding partners to the test piece. If the test piece is silicon or glass, the surface must be activated prior to attaching the specific binding partner. Also, polyelectrolyte interactions may be used to immobilize a specific binding partner on a surface of a test piece by using techniques and chemistries. The preferred method of attachment is by covalent means. Following attachment of a specific binding member, the surface may be further treated with materials such as serum, proteins, or other blocking agents to minimize non-specific binding. The surface also may be scanned either at the site of manufacture or point of use to verify its suitability for assay purposes. The scanning process is not anticipated to alter the specific binding properties of the test piece.

While the present invention discloses the preference for the use of solid phases, it is contemplated that the reagents such as antibodies, proteins and peptides of the present invention can be utilized in non-solid phase assay systems. These assay systems are known to those skilled in the art, and are considered to be within the scope of the present invention.

It is contemplated that the reagent employed for the assay can be provided in the form of a test kit with one or more containers such as vials or bottles, with each container containing a separate reagent such as a probe, primer, monoclonal antibody or a cocktail of monoclonal antibodies, or a polypeptide (e.g. recombinantly, synthetically produced or purified) employed in the assay. Other components such as buffers, controls and the like, known to those of ordinary skill in art, may be included in such test kits. It also is contemplated to provide test kits which have means for collecting test samples comprising accessible body fluids, e.g., blood, cerebral spinal fluid, urine, saliva and stool. Such tools useful for collection (‘collection materials’) include lancets and absorbent paper or cloth for collecting and stabilizing blood; swabs for collecting and stabilizing saliva; cups for collecting and stabilizing urine or stool samples. Collection materials, papers, cloths, swabs, cups and the like, may optionally be treated to avoid denaturation or irreversible adsorption of the sample. The collection materials also may be treated with or contain preservatives, stabilizers or antimicrobial agents to help maintain the integrity of the specimens. Test kits designed for the collection, stabilization and preservation of test specimens obtained by surgery or needle biopsy are also useful. It is contemplated that all kits may be configured in two components which can be provided separately; one component for collection and transport of the specimen and the other component for the analysis of the specimen. The collection component, for example, can be provided to the open market user while the components for analysis can be provided to others such as laboratory personnel for determination of the presence, absence or amount of analyte. Further, kits for the collection, stabilization and preservation of test specimens maybe configured for use by untrained personnel and may be available in the open market for use at home with subsequent transportation to a laboratory for analysis of the test sample.

As the man skilled in the art will readily appreciate, above considerations directed to Immunoassays are readily applicable mutatis mutandis also to tse-infected T-cells. This is an important aspect of the invention since T-cells have been shown to be the carriers of secondary infectivity.

The present invention will now be described by way of examples, which are meant to illustrate, but not to limit, the scope of the present invention.

### EXAMPLES

Examples 1-2 deal with the experimental protocol used for obtaining the results shown in tables 1 and 2. Example 3 refers to the FACS analysis shown in FIG. 2D. Examples 4-9 deal with the production of specific antibodies directed to tse-infected B-cells or directed to tse-infected T-cells. Example 10 relates to the identification of tse-infection sustaining cell types within the LRS. Example 11 was designed to investigate the interaction between tse-infected B-cells and T-cells. Example 12 relates to a new assay method contemplated by the invention. Example 13 shows the manufacture of safe, non tse-infective blood derived products as contemplated by the invention. Examples 14-18 show the therapeutical advantages achievable by the invention.

### Example 1

### Generation of t11 μMT Mice

The V-gene segment of the immunoglobulin heavy chain of the B-cell hybridoma VI41 (ref. 27) secreting a VSV-neutralizing antibody was cloned into an expression vector encoding the mouse β-chain of allotype a. Transgenic mice were generated and backcrossed to μMT mice. t11 μMT mice exclusively expressed the transgenic β-chain of the allotype a; endogenous IgM of the allotype b and immunoglobulins of other subclasses were not detected in their serum (not shown).

### Example 2

### 2.1. Scrapie Inoculation

Mice were inoculated with a 1% homogenate of heat and sarcosyl-treated brain prepared from mice infected with the Rocky Mountain laboratory (RML) scrapie strain. Thirty microliters were used for intra-cranial (i.c.) injection, whereas 100 μl were administered by intra-peritoneal (i.p.) route. Mice were monitored every second day, and scrapie was diagnosed according to standard clinical criteria.

### 2.2. Western-Blot Analysis

Ten percent brain homogenates were prepared as described 1 and, where indicated, digested with 20 μg/ml of proteinase K for 30 minutes at 37° C. Eighty μg of total protein were then electrophoresed through 12% SDS-polyacrylamide gel, transferred to nitrocellulose membranes, probed with monoclonal antibody 6H4. (Prionics AG, Zurich) or polyclonal antiserum IB3 (reference 26) against mouse PrP, and developed by enhanced chemiluminescence.

### 2.3. Detection of PrP Antibodies

Brain Lysates from wild-type and Prnp^{o/o} mice, as well as recombinant E. coli PrP, were electrophoresed through a 12.5% SDS-polyacrylamide gel and transferred to nitrocellulose membranes. Membranes were then incubated with serum from infected, terminally scrapie-sick mice (1:100 diluted). Visualization was achieved by enhanced chemiluminescence as previously described for the Western-blot.

### 2.4. Immunohistochemical Studies

Brain tissue from each mouse was fixed, inactivated for 1 hour with 98% formic acid, embedded in paraffin and subjected to conventional staining and to immuno-staining for glial fibrillary acidic protein according to standard procedure. Gliosis (a nonspecific but early indicator of brain damage) was detected by the presence of large immunostained reactive astrocytes. In terminally scrapie-sick mice, wide spread vacuolation was consistently seen throughout the central nervous system.

### 2.5. Infectivity Bioassays

Brain and spleen homogenates (w/v, 10% in 0.32 M sucrose) were prepared from infected animals as described, and 30 μl (diluted 1:10 in phosphate buffered saline containing 1% BSA) were administered i.c. to groups of at least 4 tga²⁰ mice for each sample. The incubation time until development of terminal scrapie sickness was determined and infectivity titers were calculated using the relationship y=14.37 0.11x where y is the ID₅₀ and x is the incubation time (in days) to terminal disease.

### 2.6. Preparation of Splenocytes

Spleens were recovered from mice at 34 days following i.p. inoculation with the RML strain of prions. Splenocyte suspensions were prepared by forcing spleens through a fine mesh screen into 25 ml of magnetic activated cell separation (MACS) buffer. The MACS buffer is composed of phosphate buffered saline containing 1% BSA, 5 mM EDTA and 0.1% sodium azide. Following a 15 minute incubation on ice to allow the cell clumps to settle the cell suspension was removed for further evaluation.

### 2.7. Antibodies

Antibodies conjugated to super-paramagnetic microbeads which specifically recognized B and T-cells (anti-mouse-B220, anti-Thy 1.2, anti-IgM, and anti-CD3) were obtained from Milteny Biotech GmbH. All magnetic separation columns (A2 & CS Column) were also obtained from Milteny Biotech GmbH. Rabbit complement was obtained from Cedarlane, Ontario (Low-tox-M rabbit complement). Additional antibodies (LR1, mouse anti-mouse thy 1.2) were obtained from Serotec.

### 2.8. B and T-Cell Purification by Magnetic Bead Separation

Five ml of a splenocyte suspension was centrifuged at 100 rpm for 10 minutes and the cell pellet was recovered in ≈0.6 ml of MACS buffer. The cells were then incubated with 75 μl of B-220 or thy 1.2 conjugated super-paramagnetic microbeads as per manufacturer instruction (Milteny Biotech GmbH) for 15 minutes at 4° C. Following the incubation, the total volume was adjusted to 2 ml with MACS buffer and loaded onto a prefilled and washed A2 column (magnetic separation column). Cells not associated with the magnetic microbeads were eluted with 5 ml of MACS buffer. The column was then removed from the magnetic field and back flushed to remove the extracted cells. The separation process is then repeated and the final B or T enriched cell population is eluted with 11 ml of MACS buffer after the separation column was removed from the magnetic field.

### 2.9. Complement Lysis

To further improve the purity of the B and T cell population obtained by magnetic separation, complement lysis of the T or B cell enriched population was performed. Cells were pelleted and resuspended in cytotoxicity medium (CM, RPMI-1640 media containing 25 mM HEPES and 0.3% BSA) to a concentration of 1−3×10⁷ cells/ml. For B cell depletion, a B cell specific antibody, e.g., LR1 was used. Whereas for T cell depletion, a T cell specific antibody, e.g., Thy 1.2 was used. Optimal effective antibody concentration would need to be individually determined for the specific antibody sources. Incubation with the antibodies is performed at 4° C. for 60 minutes after which the cells were resuspended in LCM containing 20% Low-tox-M rabbit complement and incubated at 37° C. for 60 minutes to allow for cell lysis. Viable cells were then separated from the dead cells and debris by centrifugation over lympholyte-M (Cedarlane, Ontario) or other cell separation medium according to the manufacturer's instruction.

### 2.10. Cell Preparation for Flow Cytometry Analysis

Single cell suspension for flow cytometry analysis were prepared in FACS buffer consisting of phosphate buffered saline containing 2% FCS, 20 mM EDTA and 1% sodium azide. When peripheral blood samples were used, the lymphocyte population was enriched by lysis removal of the red blood cells from heparinized blood. The cell staining process consists of incubating cell population with saturating concentration of fluorescein (FITC)-conjugated antibodies for 30 minutes at 4° C. The cells were then washed with FACS buffer to remove the unbounded material and subject to flow analysis. When the indirect staining method was used, the cell populations were first incubated with the primary antibody for 30 minutes at 4° C., washed with FACS buffer and followed with an additional 30 minutes of incubation at 4° C. with a secondary FITC-conjugated antibody. After removal of the unbounded FITC-conjugated secondary antibodies, the cell populations were then ready for flow analysis.

Discussion of the Results of Example 2

1. Determination of Scrapie Infectivity

Infectivity of brain material from scrapie infected mice was demonstrated by i.e. infection of tga20 indicator mice. Infectivity was determined by injecting 30 μl samples i.c. into tga20 mice and determining time to disease manifestation by standard histochemical procedure. Table 5 illustrates a typical outcome of such analysis. This analysis gives the success rate of disease transmission and the duration/incubation time for the expression of the disease symptoms. Hence the assays reveal the susceptibility of the host strain to the disease and, thus allow for the determination of the critical cell types necessary for disease transmission.

TABLE 5
TABLE 5 Determination of scraple infectivity

| Source of | | | Incubation |
|---|---|---|---|
| Inactivity | | | time of |
| (a) Standard | Days after | Trans- | recipient |
| prion inoculum† | Inoculation | mission* | (days) |
| RML, 10⁻¹ | — | 4/4 | 59, 60, 63, 68 |
| RML, 10⁻³ | — | 2/2 | 66, 67 |
| RML, 10⁻⁵ | — | 4/4 | 84, 85, 85, 96 |
| RML, 10⁻⁷ | — | 1/3 | 109, >217, >217 |
| RML, 10⁻⁹ | — | 0/4 | >217, >217, >217, >217 |
| RML, 10⁻¹¹ | — | 0/3 | >217, >217, >217 |

2. Evaluation of the Potential Target Cells for Scrapie Transmission by Genetic Methodology

The effect of immune defects on the pathogenesis of scrapie was studied in mice deficient in T cells, B cells or with combined T/B cell defects. A number of different mouse genotypes that are suitable have been generated and the selection of the type to be used will be apparent to a person skilled in the art. The success of infection is determined by examination of the disease symptoms, pathology and by infectivity bioassay. Table 1 illustrates a typical outcome of such analysis. This analysis gives the incubation time from infection to symptom presentation, the presence or absence of symptoms and pathological features. Further the infectivity bioassay provides information regarding the latency of the infective agents in the brain and splenic tissues of the primary infected host. By correlating the disease expression and genotype of infected animals, table 1 illustrates that if the infective agent is introduced by the i.c. route all genotypes express the disease regardless of their B cell or T cell defects. Alternatively, by examining the (secondary) infective capability of brain and splenic tissues from the primary infected hosts, the potential target cell lineage of scrapie transmission can be examined. Thus table 2 further illustrates that following i.c. inoculation, only those genotypes with intact B cell functions are capable of demonstrating secondary infectivity in the spleen tissues.

By taking a more peripheral route of primary infection, i.c., i.p. inoculation, the propagation of the disease can be further delineated. This is further illustrated in tables 1 and 2. The analysis demonstrates that by selecting’ animals with specific lymphocyte defects, the critical lymphoid cell types for scrapie disease transmission can be specifically identified. These results suggest that B cells may “transport” prions from lymphoid organs to nervous tissues. (The mode of transport is not limited to direct cell associated transport but may also be complexes with various cellular products. The components are not limited to but may include antibodies, PrP_{c}, PrP^{sc}. and other similar cellular products).

3. Evaluation of the Role of Lymphoid Cells in Prion Disease Transmission

Cellular components of the peripheral lymphoid tissues, e.g., spleen, lymph nodes can be readily obtained from animals. Such conditions are described by public literature.

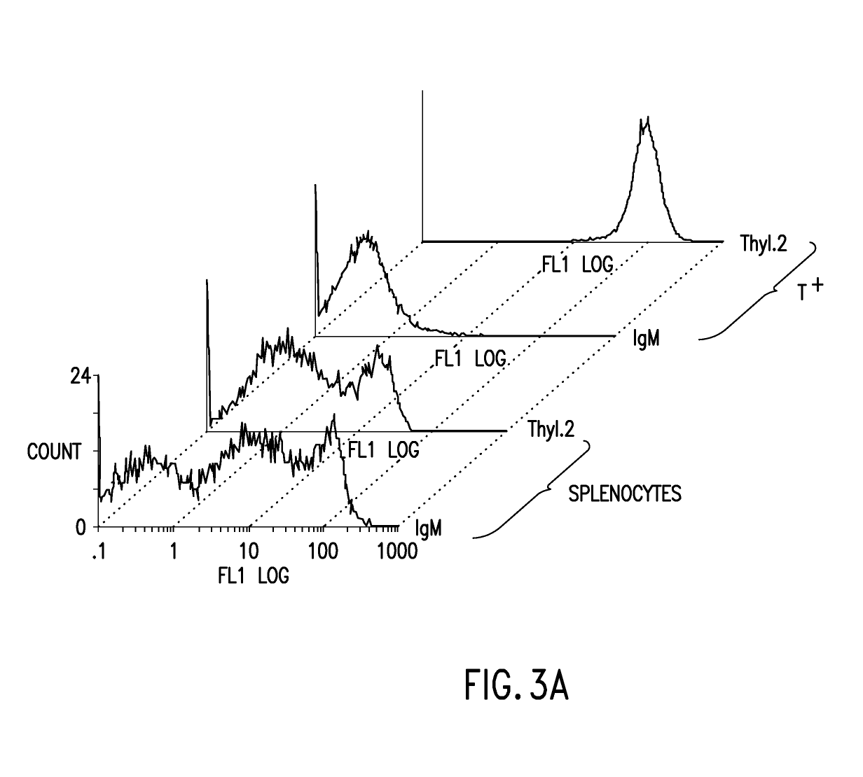

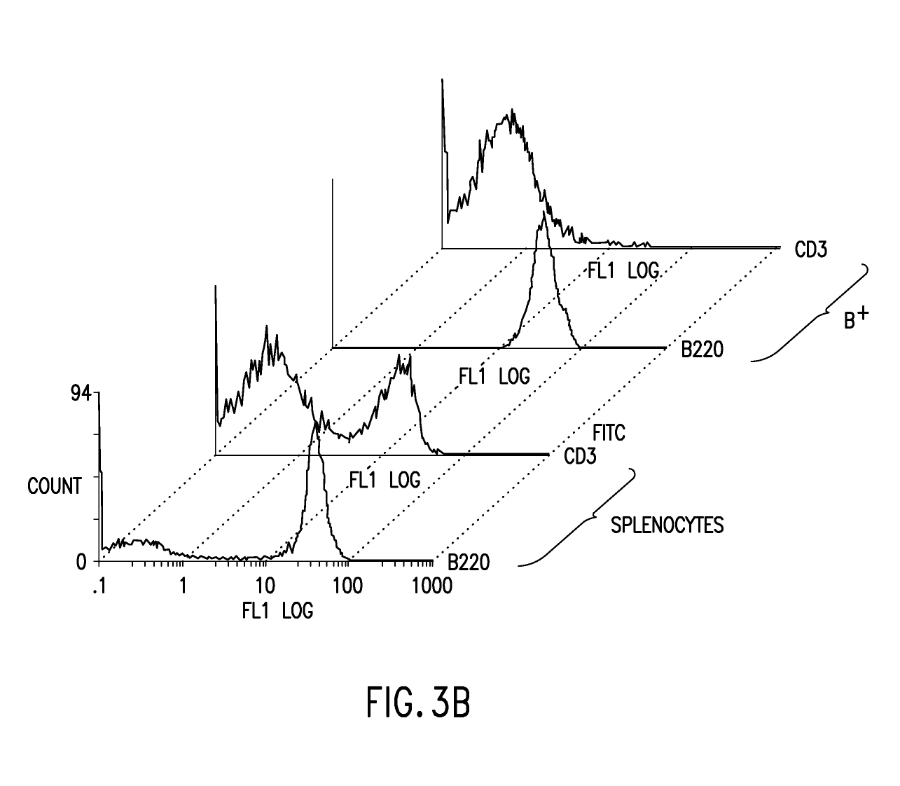

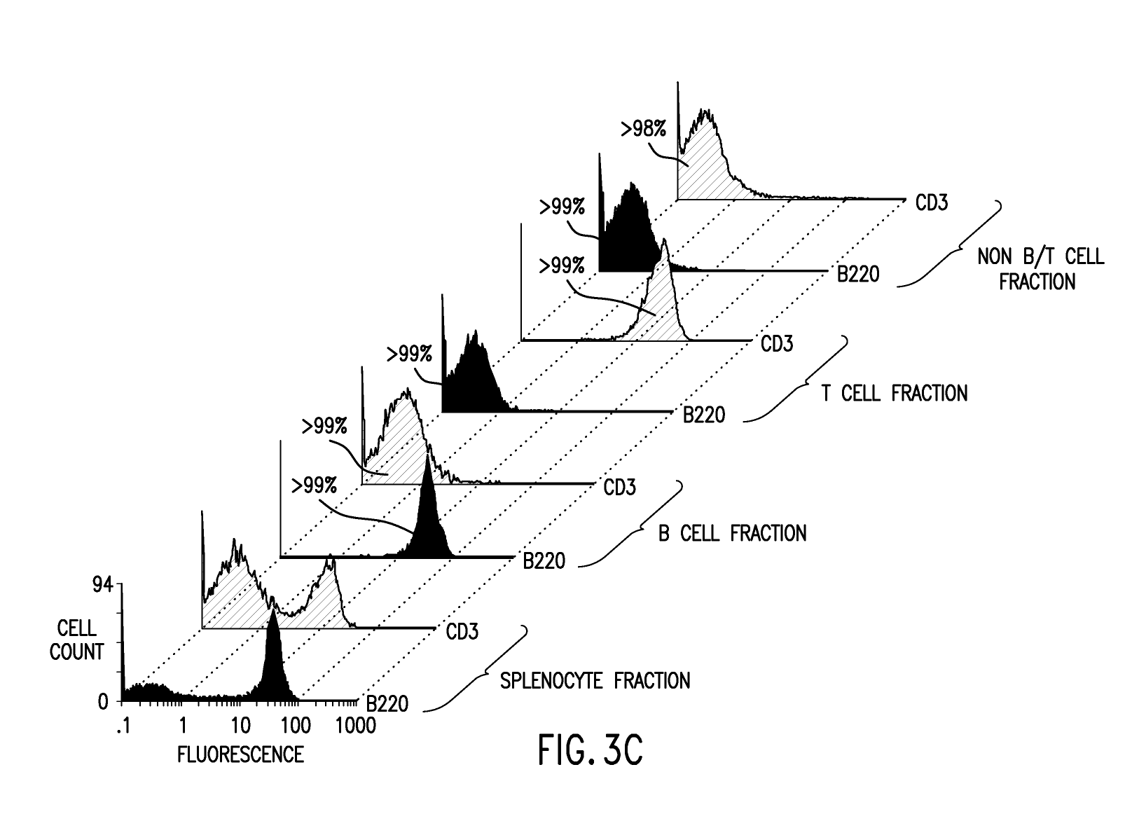

The cellular components obtained can be further separated by specific antibody to differential surface markers for the various lymphoid cell types which has been conjugated to magnetic microbeads. By additional deletion of undesirable cell types by cytotoxic depletion using complement, highly purified cell isolates can be obtained. The procedure is constructed to isolate highly enriched T-cell and B-cell populations. The isolated cell populations are suspended in culture medium, e.g., RPMI-1640 and can be supplemented with serum and with additives like glutamic acid, growth factors, cytokines or other modulators of cell physiology prior to evaluation of infectivity capacity. Such highly enriched lymphocytes can be further characterized by Flow cytometry evaluation of the membrane surface components, e.g., CD-4, CD-8, and/or Ig expression and is obvious to a person skilled in the art. FIGS. 3a and 3b illustrate a typical Flow analysis of such enriched population. The cellular purity is demonstrated by the expression of T cell or B cell specific surface markers. Other non cell lineage associated components can also be documented by similar means, e.g., cell surface expression of PrP^{c} and PrP^{sc}. Further, molecular biology techniques as described by public literature can also be employed to document non-membrane associated specific intracellular components, e.g., DNA, RNA, mRNA whose presence is indicative of its cellular presence.

Such cellular lymphoid components can be obtained from infective and non-infective hosts and characterized for its lineage and intracellular capacities. Subsequently, their infective capacity can be examined by inoculation via the i.c. or i.p. route. By this assay it is possible to determine the cell lineage most responsible for prion disease transmission. Further, by measurement of various intracellular components and correlation with the cellular lineage, the assay is indicative of the interactions between the prions and the tentative target cells.

TABLE 6
Infectivity in cell fractions of Tg94 and wt spleen

| | | Tg94 (1. Experiment) | | Tg94 (2. Experiment) | | wt | |
|---|---|---|---|---|---|---|---|
| Fraction | Cells | Inc. time | n/no | Inc. time | n/no | Inc. time | n/no |
| splenocytes | 10⁶ | 76 ± 4 | (4/4) | nd | | nd | |
| | 10⁵ | 84.5 ± 1 | (2/2) | 89 ± 1 | (2/2) | 83.5 ± 3 | (2/2) |
| | 10⁴ | 109 ± 14 | (4/4) | 106.5 ± 18 | (4/4) | 89 ± 5 | (4/4) |
| | 10³ | 142 | (1/4) | 116.5 ± 12 | (2/4) | 106 ± 7 | (4/4) |
| | 10² | 141 ± 35 | (2/2) | >200 | (0/4) | >200 | (0/4) |
| B cells | 2 × 10⁵ | 76 ± 1 | (4/4) | nd | | nd | |
| | 2 × 10⁴ | 87 ± 1 | (3/4) | 106 ± 10 | (4/4) | 88 ± 2 | (4/4) |
| | 2 × 10³ | 116 ± 37 | (3/4) | 106 ± 11 | (2/4) | 102 ± 14 | (4/4) |
| | 2 × 10² | 110 | (1/4) | >200 | (0/4) | 91 | (1/4) |
| T cells | 10⁵ | 82 ± 6 | (4/4) | nd | | nd | |
| | 10⁴ | 110 ± 10 | (3/4) | 109.5 ± 1 | (2/4) | 94 ± 8 | (4/4) |
| | 10³ | 106 | (1/3) | >200 | (0/4) | 108 ± 18 | (2/2) |
| | 10² | 108 | (1/4) | >200 | (0/4) | >200 | (0/4) |
| Non B/T cells | 5 × 10⁵ | 124.5 ± 1 | (2/4) | 108 | (1/4) | 112 ± 14 | (3/4) |
| | 5 × 10⁴ | | (0/4) | nd | | >200 | (0/4) |

### Example 3

### FACS Analysis Shown in FIG. 2D

Peripheral blood cells were incubated with serum from t11 μMT mice, washed, incubated with anti-mouse IgM-FITC conjugate followed by anti-CD3-PE (Pharmingen), and analyzed with a Becton-Dickinson FAScan instrument after erythrocyte lysis and fixation. For analysis, cells were gated on CD3-positive T-cells. EL4 cells infected with vesicular stomatitis virus (VSV) were stained with 5 μg VSV-specific monoclonal antibody VI24 (ref. 27) and with FTC-labelled antibody to mouse IgG2a (Southern Biotechnology), or with serum of t11 μMT mice, and with FITC-labelled F(ab′)2 antibody to mouse IgM (anti-IgM-FITC, Tago), or with serum of C57BL/6 mice and anti-IgM-FITC. All data acquisition and analysis were performed with CellQuest software (Becton Dickinson).

### Example 4

### Production of Antibodies Against TSE-Infected Lymphocytes

A. Production of Polyclonal Antisera.

Antiserum against tse-infected lymphocytes (i.e. B-cells or T-cells) is prepared by injecting appropriate animals with tse-infected lymphocytes identified and isolated as described in example 2.

1. Starting Materials

Specifically, purified B-cell preparations and/or T-cell preparations are used. The whole cell preparations of tse-infected lymphocytes can be used directly as. immunogen or alternatively tse-infected lymphocytes can be gently lysed with mild detergent treatment for example with 0.05-0.5% Triton X 100 followed by fixation in 0.5-2% paraformaldehyde in 1% PBS for 5-100 minutes at 4-10° C.

2. Animal Immunization.

Female white New Zealand rabbits weighing 2 kg or more are used for raising polyclonal antiserum. Generally, one animal is immunized per infective lymphocyte preparation. One week prior to the first immunization, 5 to 10 ml of blood is obtained from the animal to serve as a non-immune prebleed sample.

Tse-infected lymphocytes are used to prepare the primary immunogen by emulsifying 0.5 ml of the tse-infected lymphocyte preparation at a concentration of between 1×10⁵ to 1×10³ cells/ml in PBS (pH 7.2) with 0.5 ml of complete Freund's adjuvant (CFA) (Difco, Detroit, Mich.). The immunogen is injected into several sites of the animal via subcutaneous, intraperitoneal, and/or intramuscular routes of administration. Four weeks following the primary immunization, a booster immunization is administered. The immunogen used for the booster immunization dose is prepared by emulsifying 0.5 ml of the same tse-infected lymphocyte preparation used for the primary immunogen, except that 0.5 ml of incomplete Freund's adjuvant (IFA) (Difco, Detroit, Mich.) is now used. Again, the booster dose is administered into several sites and can utilize subcutaneous, intraperitoneal and intramuscular types of injections. The animal is bled (5 ml) two weeks after the booster immunization and the serum is tested for immunoreactivity to the tse-infected lymphocyte preparation as described below. The booster and bleed schedule is repeated at 4 week intervals until an adequate titer is obtained. The titer or concentration of antiserum is determined by microtiter EIA as described in Example 17, below. An antibody titer of 1:500 or greater is considered an adequate titer for further use and study.

B. Production of Monoclonal Antibody.

1. Immunization Protocol.

Mice are immunized using immunogens (i.e. tse-infected B-cells or T-cells) prepared as described hereinabove, except that the amount of the immunogen for monoclonal antibody production in mice is one-tenth the amount used to produce polyclonal antisera in rabbits. The primary immunogen consists of 0.1 ml of the tse-infected lymphocyte preparation at a concentration of between 1×10⁵ to 1×10⁸ cells/ml in PBS (pH 7.2) in 0.1 ml of CFA emulsion; while the immunogen used for booster immunizations consists of 0.1 ml of the tse-infected lymphocyte preparation as above emulsified with 0.1 ml of IFA. Hybridomas for the generation of monoclonal antibodies are prepared and screened using standard techniques. The methods used for monoclonal antibody development follow procedures known in the art such as those detailed in Kohler and Milstein, Nature 256:494 (1975) and reviewed in J. G. R. Hurrel, ed., Monoclonal Hybridoma Antibodies: Techniques and Applications, CRC Press, Inc., Boca Raton, Fla. (1982). Another method of monoclonal antibody development which is based on the Kohler and Milstein method is that of L. T. Mimms et al., Virology 176:604-619 (1990), which is incorporated herein by reference.

The immunization regimen (per mouse) consists of a primary immunization with additional booster immunizations. Booster immunizations are performed at approximately two weeks and four weeks post primary immunization. A total of 100 ul of immunogen is inoculated intraperitoneally and subcutaneously into each mouse. Individual mice are screened for immune response by microtiter plate enzyme immunoassay (EIA) as described in Example 17 approximately four weeks after the third immunization. Mice are inoculated” either intravenously, intrasplenically or intraperitoneally with 0.1 ml of the tse-infected lymphocyte preparation at a concentration of between 1×10⁵ to 1×10⁸ cells/ml in PBS (pH 7.2) in 0.1 ml of IFA approximately fifteen weeks after the third immunization.

Three days after this intravenous boost, splenocytes are fused with, for example, Sp2/0-Ag14 myeloma cells (Milstein Laboratories, England) using the polyethylene glycol (PEG) method. The fusions are cultured in Iscove's Modified Dulbecco's Medium (IMDM) containing 10% fetal calf serum (FCS), plus 1% hypoxanthine, aminopterin and thymidine (HAT). Bulk cultures are screened by microtiter plate EIA following the protocol in Example 17. Clones reactive with the tse-infected lymphocyte preparation used as immunogen and non-reactive with non-tse-infected lymphocyte preparation (i.e., lymphocytes prepared from non-infected animals not used as the immunogen) are selected for final expansion. Clones thus selected are expanded, aliquoted and frozen in IMDM containing 10% FCS and 10% dimethyl-sulfoxide.

2. Production of Ascites Fluid Containing

Monoclonal Antibodies.

Frozen hybridoma cells prepared as described hereinabove are thawed and placed into expansion culture. Viable hybridoma cells are inoculated intraperitoneally into Pristane treated mice. Ascites fluid is removed from the mice, pooled, filtered through a 0.2u filter and subjected to an immunoglobulin class G (IgG) analysis to determine the volume of the Protein A column required for the purification.

3. Purification of Monoclonal Antibodies From Ascites Fluid.

Briefly, filtered and thawed ascites fluid is mixed with an equal volume of Protein A sepharose binding buffer (1.5 M glycine, 3.0 M NaCl, pH 8.9) and refiltered through a 0.2μ filter. The volume of the Protein A column is determined by the quantity of IgG present in the ascites fluid. The eluate then is dialyzed against PBS (pH 7.2) overnight at 2-8° C. The dialyzed monoclonal antibody is sterile filtered and dispensed in aliquots. The immunoreactivity of the purified monoclonal antibody is confirmed by determining its ability to specifically bind to the tse-infected lymphocyte preparation used as the immunogen by use of the EIA microtiter plate assay procedure of Example 17. The specificity of the purified monoclonal antibody is confirmed by determining its lack of binding to irrelevant non tse-infected lymphocytes not used as the immunogen. The purified anti tse-infected lymphocyte monoclonal thus prepared and characterized is placed at either 2-8° C. for short term.’ storage or at −80° C. for long term storage.

4. Further Characterization of Monoclonal Antibody.

The isotype and subtype of the monoclonal antibody produced as described hereinabove can be determined using commercially available kits (available from Amersham, Inc., Arlington Heights, Ill.). Stability testing also can be performed on the monoclonal antibody by placing an aliquot of the monoclonal antibody in continuous storage at 2-8° C. and assaying optical density (OD) readings throughout the course of a given period of time.

C. Use of Recombinant Proteins as Immunogens.

It is within the scope of the present invention that recombinant proteins made as described herein can be utilized as immunogens in the production of polyclonal and monoclonal antibodies, with corresponding changes in reagents and techniques known to those skilled in the art.

### Example 5

### Purification of Serum Antibodies which Specifically Bind to Tse-Infected Lymphocytes

Immune sera, obtained as described hereinabove in Example 4, is affinity purified using immobilized proteins from the tse-infected lymphocyte preparation used as the immunogen as described above. An IgG fraction of the antiserum is obtained by passing the diluted, crude antiserum over a Protein A column (AFFI-GEL® protein A, Bio-Rad, Hercules, Calif.). Elution with a buffer (Binding Buffer, supplied by the manufacturer) removes substantially all proteins that are not immunoglobulins. Elution with 0.1 M buffered glycine (pH 3) gives an immunoglobulin preparation that is substantially free of albumin and other serum proteins.

Immunoaffinity chromatography is performed to obtain a preparation with a higher fraction of specific antigen-binding antibody. The tse-infected lymphocyte preparation used to raise the antiserum is immobilized on a chromatography resin, and the specific antibodies directed against its epitopes are adsorbed to the resin. After washing away non-binding components, the specific antibodies are eluted with 0.1 M glycine buffer, pH 2.3. Antibody fractions are immediately neutralized with 1.0M Tris buffer (pH 8.0) to preserve immunoreactivity. A resin such as AFFI-GEL® 10 or AFFI-GEL® 15 is used (Bio-Rad, Hercules, Calif.). If coupling through a carboxy is desired, AFFI-GEL® 102 can be used (Bio-Rad, Hercules, Calif.). An organomercurial resin such as AFFI-GEL® 501 can be used (Bio-Rad, Hercules, Calif.).

Alternatively, spleens can be harvested and used in the production of hybridomas to produce monoclonal antibodies following routine methods known in the art as described hereinabove.

### Example 6

### Western Blotting of Tissue Samples

Protein extracts are prepared by homogenizing tissue samples in 0.1 M Tris-HCl (pH 7.5), 15% (w/v) glycerol, 0.2 mM EDTA, 1.0 mM 1,4-dithiothreitol, 10 μg/ml leupeptin and 1.0 mM phenylmethylsulfonylfluoride (Kain et al., Biotechniques, 17:982 (1994)). Following homogenization, the homogenates are centrifuged at 4° C. for 5 minutes to separate supernate from debris. For protein quantitation, 3-10 μl of supernate are added to 1.5 ml of bicinchoninic acid reagent (Sigma, St. Louis, Mo.), and the resulting absorbance at 562 nm is measured.

For SDS-PAGE, samples are adjusted to desired protein concentration with Tricine Buffer (Novex, San Diego, Calif.), mixed with an equal volume of 2× Tricine sample buffer (Novex, San Diego, Calif.), and heated for 5 minutes at 100° C. in a thermal cycler. Samples are then applied to a Novex 10-20% Precast Tricine Gel for electrophoresis. Following electrophoresis, samples are transferred from the gels to nitrocellulose membranes in Novex Tris-Glycine Transfer buffer. Membranes are then probed with specific anti tse-infected lymphocyte antibodies using the reagents and procedures provided in the Western Lights or Western Lights Plus (Tropix, Bedford, Mass.) chemiluminescence detection kits. Chemiluminescent_bands are visualized by exposing the developed membranes to HYPERFILM™ ECL™ (Amersham, Arlington Heights, Ill.).

Competition experiments are carried out in an analogous manner as above, with the following exception; the primary antibodies (anti tse-infected lymphocyte polyclonal antisera) are pre-incubated for 30 minutes at room temperature with varying concentrations of non tse-infected lymphocyte immunogen prior to exposure to the nitrocellulose filter. Development of the Western is performed as above.

After visualization of the bands on film, the bands can also be visualized directly on the membranes by the addition and development of a chromogenic substrate such as 5-bromo-4-chloro-3-indolyl phosphate (BCIP). This chromogenic solution contains 0.016% BCIP in a solution containing 100 mM NaCl, 5 mM MgCl₂ and 100 mM Tris-HCl (pH 9.5). The filter is incubated in the solution at room temperature until the bands develop to the desired intensity. Molecular mass determination is made based upon the mobility of pre-stained molecular weight standards (Novex, San Diego, Calif.) or biotinylated molecular weight standards (Tropix, Bedford, Mass.).

### Example 7

### EIA Microtiter Plate Assay

The immunoreactivity of antiserum preferably obtained from rabbits or mice as described in Example 4 is determined by means of a microtiter plate EIA, as follows. Protein from tse-infected or non-tse-infected lymphocyte preparations as described above is prepared by homogenization of lymphocytes in an appropriate buffer for example PBS (7.2) or with a mild detergent such as 0.01% Triton X 100. Next, 100 ul of the above protein solution is placed in each well of an IMMULON 2® microtiter plate (Dynex Technologies, Chantilly, Va.). The plate is incubated overnight at room temperature and then washed four times with deionized water. The wells are blocked by adding 125 μl of a suitable protein blocking agent, such as SUPERBLOCK™ (Pierce Chemical Company, Rockford, Ill.), in phosphate buffered saline (PBS, pH 7.4) to each well and then immediately discarding the solution. This blocking procedure is performed three times. Antiserum obtained from immunized rabbits or mice prepared as previously described is diluted in a protein blocking agent (e.g., a 3% SUPERBLOCK™ solution) in PBS containing 0.05% Tween-20 (monolaurate polyoxyethylene ether) (Sigma Chemical Company, St. Louis, Mo.) and 0.05% sodium azide at dilutions of 1:500, 1:2500, 1:12,500, 1:62,500 and 1:312,500 and placed in each well of the coated microtiter plate. The wells then are incubated for three hours at room temperature. Each well is washed four times with deionized water. One hundred ul of alkaline phosphatase-conjugated goat anti-rabbit IgG or goat anti-mouse IgG antiserum (Southern Biotech, Birmingham, AB), diluted 1:2000 in 3% SUPERBLOCK™ solution in phosphate buffered saline containing 0.05% Tween 20® and 0.05% sodium azide, is added to each well. The wells are incubated for two hours at room temperature. Next, each well is washed four times with deionized water. One hundred microliters (100 μl) of paranitrophenyl phosphate substrate (Kirkegaard and Perry Laboratories, Gaithersburg, Md.) then is added to each well. The wells are incubated for thirty minutes at room temperature. The absorbance at 405 nm is read of each well. Positive reactions are identified by an increase in absorbance at 40 5 nm in the test well above that absorbance given by a non-immune serum (negative control). A positive reaction is indicative of the presence of detectable anti tse-infected lymphocyte antibodies.

In addition to titers, apparent affinities [K_{d}(app)] may also be determined for some of the antisera. EIA microtiter plate assay results can be used to derive the apparent dissociation constants (K_{d}) based on an analog of the Michaelis-Menten equation (V. Van Heyningen, Methods in Enzymology, Vol. 121, p. 472 (1986) and further described in X. Qiu, et al., Journal of Immunology, Vol. 156, p. 3350 (1996)).

### Example 8

### Coating of Solid Phase Particles

A. Coating of Microparticles with Antibodies which Specifically Bind to Tse-infected Lymphocytes.

Affinity purified antibodies which specifically bind to tse-infected lymphocytes (see Example 5) are coated onto microparticles of polystyrene, carboxylated polystyrene, polymethylacrylate or similar particles having a radius in the range of about 0.1 to 20 urn. Microparticles may be either passively or actively coated. One coating method comprises coating EDAC (1-(3-dimethylaminopropyl)-3-ethylcarbodiimide hydrochloride (Aldrich Chemical Co., Milwaukee, Wis.) activated carboxylated latex microparticles with antibodies which specifically bind to tse-infected lymphocytes, as follows. Briefly, a final 0.375% solid suspension of resin washed carboxylated latex microparticles (available from Bangs Laboratories, Carmel, Ind. or Serodyn, Indianapolis, Ind.) are mixed in a solution containing 50 mM MES buffer, pH 4.0 and 150 mg/l of affinity purified anti tse-infected lymphocyte antibody (see Example 4) for 15 min in an appropriate container. EDAC coupling agent is added to a final concentration of 5.5 μg/ml to the mixture and mixed for 2.5 h at room temperature.

The microparticles then are washed with 8 volumes of a Tween 20®/sodium phosphate wash buffer (pH 7.2) by tangential flow filtration using a 0.2 μm Microgon Filtration module. Washed microparticles are stored in an appropriate buffer which usually contains a dilute surfactant and irrelevant protein as a blocking agent, until needed.

B. Coating of ¼ Inch Beads.

Antibodies which specifically bind to tse-infected lymphocyte antigen also may be coated on the surface of ¼ inch polystyrene beads by routine methods known in the art (Snitman et al, U.S. Pat. No. 5,273,882, incorporated herein by reference) and used in competitive binding or EIA sandwich assays.

Polystyrene beads first are cleaned by ultrasonicating them for about 15 seconds in 10 mM NaHCO₃ buffer at pH 8.0. The beads then are washed in deionized water until all fines are removed. Beads then are immersed in an antibody solution in 10 mM carbonate buffer, pH 8 to 9.5. The antibody solution can be as dilute as 1 μg/ml in the case of high affinity monoclonal antibodies or as concentrated as about 500 μg/ml for polyclonal antibodies which have not been affinity purified. Beads are coated for at least 12 hours at room temperature, and then they are washed with deionized water. Beads may be air dried or stored wet (in PBS, pH 7.4). They also may be overcoated with protein stabilizers (such as sucrose) or protein blocking agents used as non-specific binding blockers (such as irrelevant proteins, Carnation skim milk, SUPERBLOCK™, or the like).

### Example 9

### Microparticle Enzyme Immunoassay (MEIA)

Tse-infected lymphocyte antigens are detected in patient test samples by performing a standard antigen competition EIA or antibody sandwich EIA and utilizing a solid phase such as microparticles (MEIA). The assay can be performed on an automated analyzer such as the IMx® Analyzer (Abbott Laboratories, Abbott Park, Ill.).

A. Antibody Sandwich EIA.

Briefly, samples suspected of containing tse-infected lymphocyte antigen are incubated in the presence of anti lymphocyte antibody-coated microparticles (prepared as described in Example 7) in order to form antigen/antibody complexes. The microparticles then are washed and an indicator reagent comprising an antibody conjugated to a signal generating compound (i.e., enzymes such as alkaline phosphatase or horseradish peroxidase) is added to the antigen/antibody complexes or the microparticles and incubated. The microparticles are washed and the bound antibody/antigen/antibody complexes are detected by adding a substrate (e.g., 4-methyl umbelliferyl phosphate (MUP), or OPD/peroxide, respectively), that reacts with the signal generating compound to generate a measurable signal. An elevated signal in the test sample, compared to the signal generated by a negative control, detects the presence of tse-infected lymphocyte antigen. The presence of tse-infected lymphocyte antigen in the test sample is indicative of a diagnosis of transmissible spongiform encephalopathy (TSE).

B. Competitive Binding Assay.

The competitive binding assay uses a protein or proteins from a tse-infected lymphocyte preparation that generates a measurable signal when the labeled protein is contacted with an anti tse-infected lymphocyte antibody coated microparticle. This assay can be performed on the IMx® Analyzer (available from Abbott Laboratories, Abbott Park, Ill.). The labeled proteins from a tse-infected lymphocyte preparation are added to the tse-infected lymphocyte antibody-coated microparticles (prepared as described in Example 7) in the presence of a test sample suspected of containing tse-infected lymphocyte antigen, and incubated for a time and under conditions sufficient to form labeled tse-infectived lymphocyte protein/bound antibody complexes and/or patient tse-infected lymphocyte antigen/bound antibody complexes. The tse-infected lymphocyte antigen in the test sample competes with the labeled tse-infected lymphocyte proteins for binding sites on the microparticle. Tse-infected lymphocyte antigen in the test sample results in a lowered binding of labeled infective lymphocyte protein and antibody coated microparticles in the assay since antigen in the test sample and the tse-infected lymphocyte protein compete for antibody binding sites. A lowered signal (compared to a control) indicates the presence of tse-infected lymphocyte antigen in the test sample. The presence of tse-infected lymphocyte antigen suggests the diagnosis of TSE.

The tse-infected lymphocyte proteins discussed hereinabove are useful as markers of TSE. Tests based upon the appearance of this marker or markers in a test sample such as blood, serum, plasma, cerebral spinal fluid, and tissues can provide low cost, non-invasive, diagnostic information to aid the physician to make a diagnosis of TSE, to help select a therapy protocol, or to monitor the success of a chosen therapy. This marker or markers may appear in readily accessible body fluids such as blood, urine, CSF, or stool as antigens derived from the diseased tissue which are detectable by immunological methods. This marker may be elevated in a disease state, altered in a disease state, or be a normal protein which appears in an inappropriate body compartment, in an altered state or form indicative of disease.

### Example 10

### Experimental Design Showing Susceptibility of B- and T-Cells for Transmissible Spongiform Encephalopathy

Determination of scrapie infectivity in fractionated splenocytes of different genotypes.

In a first experiment, spleens of wild-type (129/Sv-C57BL/6) mice 34 days after i.p. inoculation with RML prions are analyzed. B and T cells are purified from the spleen by magnetic activated cell sorting (MACS) followed by complement lysis of B cells in the T cell fraction and vice versa. Finally, viable cells are isolated by density gradient centrifugation. This three-step procedure leads consistently to highly purified T and B cell preparations devoid of detectable cross-contamination, as shown by FACS analysis (FIGS. 3a-c), in 5-10% yield. In addition, a non-B, non-T cell population is obtained by depleting splenocytes of B and T cells by MACS; this fraction contains ≦2% T but no detectable B-lymphocytes. The cell preparations are analyzed for infectivity by endpoint titration (Table 7). Total, splenocytes have about 3.5 log LD₅₀ units per 10⁶ cells and both B and T cells show infectivity titers within the same order of magnitude, 3.4 and 3.5 log LD₅₀ units per 10⁶ cells, respectively.

TABLE 7
Table 1. Infectivity in spleen cell fractions and PBLs of scrapie-infected mice*

| | Cell number | tg94/IRF | | Prnp^{+/+} | | Prnp+/+[FL C-Prnp^{0/0}] | |
|---|---|---|---|---|---|---|---|
| Fraction | or dilution | Inc. time | n/no | Inc. time | n/no | Inc. time | n/no |
| splenocytes | 10⁶ | nd | | nd | | >200 | (0/4) |
| | 10⁵ | 88, 90 | (2/2) | 81, 86 | (2/2) | nd | |
| | 10⁴ | 106.5 ± 18 | (4/4) | 89 ± 5 | (4/4) | | |
| | 10³ | 108, 125 | (2/4) | 106 ± 7 | (4/4) | | |
| | 10² | >200 | (0/4) | >200 | (0/4) | | |
| B cells | 2 × 10⁵ | nd | | nd | | >200 | (0/4) |
| | 2 × 10⁴ | 106 ± 10 | (4/4) | 88 ± 2 | (4/4) | nd | |
| | 2 × 10³ | 98, 114 | (2/4) | 102 ± 14 | (4/4) | | |
| | 2 × 10² | >200 | (0/4) | 91 | (1/4) | | |
| T cells | 10⁵ | nd | | nd | | >200 | (0/4) |
| | 10⁴ | 109, 110 | (2/4) | 94 ± 8 | (4/4 | nd | |
| | 10³ | >200 | (0/4) | 95, 121 | (2/2) | | |
| | 10² | >200 | (0/4) | >200 | (0/4) | | |
| Non-B/T cells | 5 × 10⁵ | 108 | (1/4) | 112 ± 14 | (3/4) | >200 | (0/4) |
| | 5 × 10⁴ | nd | | >200 | (0/4) | nd | |

*Wild-type mice (Prnp^{+/+}), transgenic Prnp^{0/0} mice overexpressing PrP in spleen (lg94/IRF) and wild-type mice irradiated and reconstituted with FLCs from Prnp^{0/0} mice (Prmp^{+/+}[FLC-Prnp^{0/0}]) were inoculaled i.p. with RML prions, spleens were recovered after 34 days and processed as described in example 10

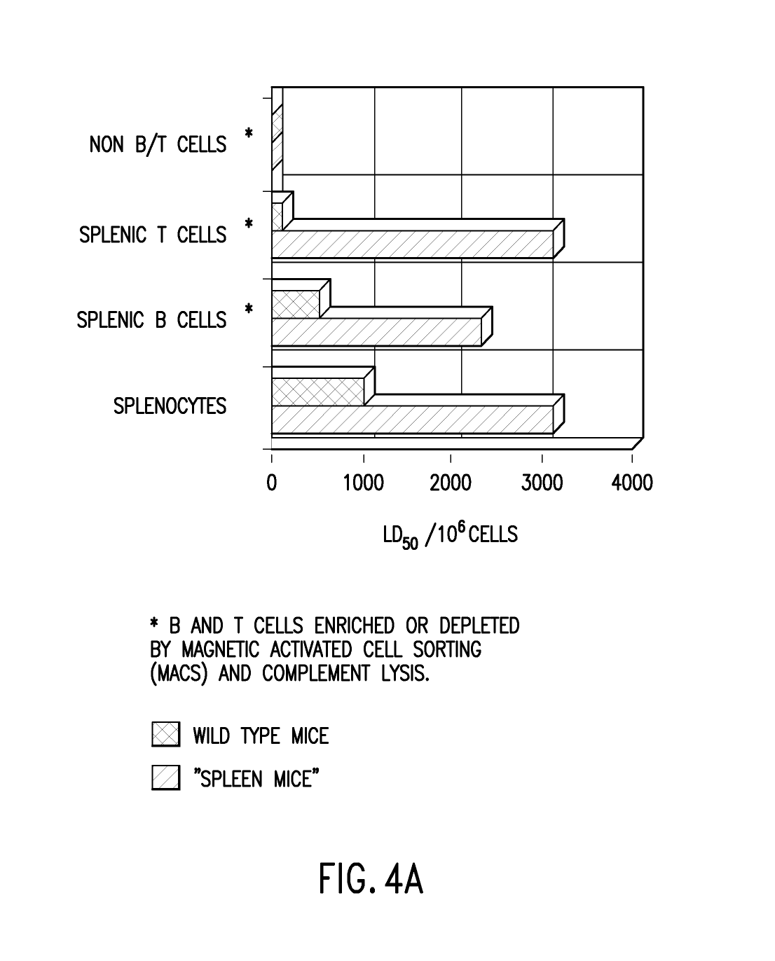

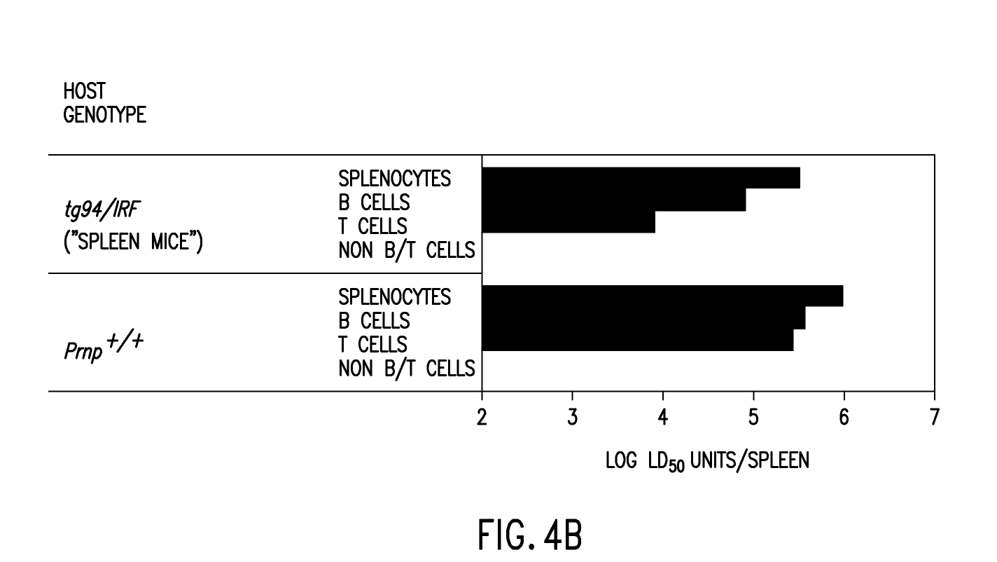

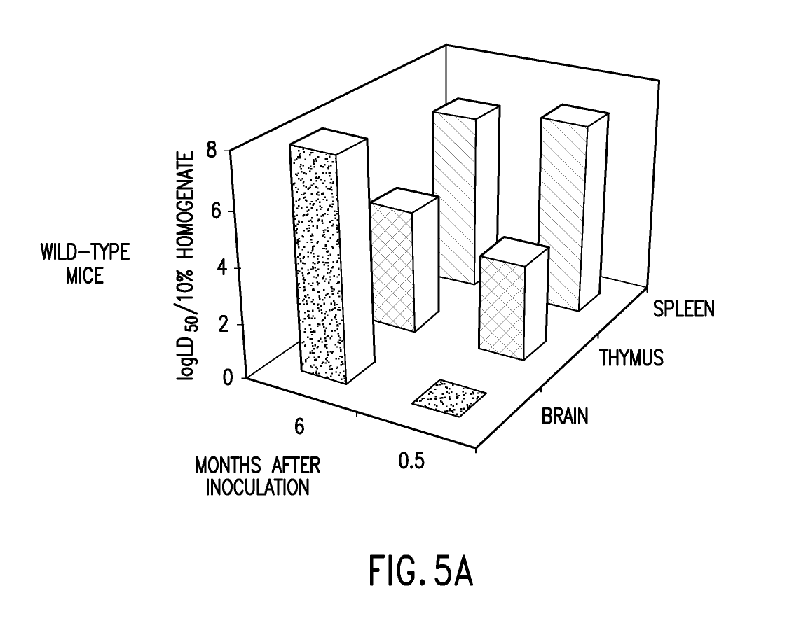

Strikingly, the non-B, non-T cell populations contain only about 1 log LD₅₀ unit per 10⁶ cells (which could be attributed to the ≦2% contamination by T lymphocytes), arguing that prion infectivity in the non B-/T-cell fraction is not due to unspecific contamination with infectivity released from elsewhere. Inasmuch as the purified B and T cells are representative of their class as regards infectivity, about 300×3.5 log LD₅₀ units=6 log LD₅₀ units of infectivity are associated with one spleen (FIG. 4b). That is to say, essentially all infectivity detected in total spleen extracts is accounted for by the fractions.

In a second similar experiment, the results of the infectivity measurements are verified using transgenic mice designated tg94/IRF. These mice contain a transgene cluster consisting of the PrP coding region under the control of a hybrid, immunoglobulin heavy-chain enhancer/IRF-1 promoter which leads to overexpression of PrP in the spleen (“spleen mice”, see also example 11). Thirty-four days after i.p. infection, tg94/IRF mice have similar levels of infectivity as wild-type mice in both, the non B-/T-cell fraction and the purified B and T cells. (Tables 6 and 7 and FIG. 4b).

Above two experiments show that in the spleen of intraperitoneally scrapie-infected wild-type mice as well as in “spleen mice”, prions are associated with B- and T-cells. In order to assess whether the association of infectivity with B-cells and T-cells is specific or adventitious, PrnP^{+/+} mice are lethally irradiated and reconstituted with FLCs derived from PrnP^{o/o} mice. PCR analysis of splenocytes confirms that these mice have undergone successful reconstitution and FACS analysis of lymphocytes demonstrates the PrnP^{o/o} origin of these cells (data not shown). Spleens from these mice, 34 days after i.p. inoculation with RML prions are fractionated and analyzed: No infectivity is found in either total splenocytes (<1 LD₅₀ unit per 10⁶ cells), or in purified B or T cells (<1 LD₅₀ unit per 10⁵ cells). This last experiment shows that splenic B and T cells devoid of PrP fail to produce or take up infectivity.

### 10.1. Scrapie Infection

RML is a mouse-adapted scrapie isolate (Chandler, R. L., Encephalopathy in mice produced by inoculation with scrapie brain material. Lancet 1, 1378-1379 (1961). It was passaged in Swiss CD-1 mice obtained from Charles River Laboratories. Inocula are 10% (w/v) homogenates of RML-infected CD-1 mouse brains in 0.32 M sucrose. Mice were infected i.p. with 100 μl of a 10-fold dilution of the inoculum in phosphate-buffered saline (PBS) containing 5% bovine serum albumin (BSA).

### 10.2. Bone Marrow Reconstitution

8 week old Prnp^{+/+} mice (129/Sv×C57BL6) were lethally irradiated and reconstituted with fetal liver cells (FLCs) from E14.5-15.5 Prnp^{o/o} (129/Sv×C57BL/6) embryos as described (Blättler, T. et al. PrP-expressing tissue required for transfer of scrapie infectivity from spleen to brain. Nature 389, 69-73 (1997). The extent of reconstitution was assessed by FACS and PCR 6-8 weeks after grafting. Inoculation with mouse scrapie prions was carried out 12 weeks after reconstitution.

### 10.3 Preparation of Splenocytes

Spleens were collected from mice 34 days after i.p. inoculation with the RML strain of prions. Splenocyte suspensions were prepared in phosphate-buffered saline with 1% BSA, 5 mM EDTA and 0.01% sodium azide (MACS buffer).

### 10.4. B and T Cell Purification

Splenocytes were incubated with anti-mouse B220 or Thy1.2 antibodies conjugated with super-paramagnetic microbeads (Milteny Biotec GmbH, Germany) for 15 min at 4° C. and applied to a prefilled and washed A2 column fixed onto the VARIO MACS (Milteny Biotec GmbH). Unlabeled cells were eluted with MACS buffer using a 23-gauge syringe attached to the column outlet as flow resistor. The column was removed from the magnet and cells were backflushed using a syringe attached to the column outlet. The column was fixed to the magnet and the cell suspension was allowed to enter the column. Unlabeled cells were again rinsed out with MACS buffer. Finally the column was removed from the magnet and labeled cells were eluted by rinsing the column with MACS buffer.

### 10.4. B and T-Cell Depletion

Splenocytes were incubated with anti-mouse B220 and anti-mouse Thy1.2 antibodies conjugated with super-paramagnetic microbeads for 15 min at 4° C. and applied to a CS column fixed onto the VARIO MACS. Unlabeled cells were eluted with MACS buffer as described above. The flow-through fraction was once again loaded onto a CS column and unlabeled cells eluted in MACS buffer.

### 10.5. Complement Lysis

MACS-purified B and T-cell fractions were further purified by complement lysis of B cells in the T cell fraction and vice versa. Cells were pelleted and resuspended in RPMI-1640 with 25 mM HEPES (pH 7.4) and 0.3% BSA (cytotoxicity medium (CM)) to give 1−3×10⁷ cells/ml. For B cell depletion, cells were incubated with a 1:200 dilution of rat anti-mouse LR1 antibody (clone LR6.2B6D6.C9, Serotec). For T cell depletion, cells were incubated with a 1:400 dilution of mouse anti-mouse Thy1.2 antibody (clone 57D5, Serotec) at 4° C. for 60 min. The cells were resuspended to the original density in CM containing 20% Low-tox-M rabbit complement (Cedarlane, Ontario) and incubated for 60 min at 37° C. Viable lymphocytes were separated from dead cells and debris by centrifugation over Lympholyte-M as recommended by the manufacturer (Cedarlane, Ontario).

10.6. FACS Analysis.

Single-cell suspensions were prepared in PBS, 2% fetal calf serum, 20 mM EDTA, 0.01% sodium azide (FACS buffer). For flow cytometry, cells were stained with saturating concentrations of fluorescein-conjugated antibodies (1 μg/10⁶ cells) for 30 min at 4° C. and washed in FACS buffer. Data acquisition and analysis were performed with an EPICS XL (Coulter) flow cytometer. Dead cells were gated out by forward and side scatter properties. Monoclonal antibodies used were fluorescein (FITC)-conjugated RA3-6B2 (B220) (GIBCO) and fluorescein (FITC)-conjugated KT3 (CD3) (Serotec).

Discussion of the Results of Example 10

Above experiments show that in the spleen of intraperitoneally tse-infected wild-type mice, prions are associated with B and T-cells. These findings confirm that B-cell and T-cell depletion are urgently required steps in the provision of safe blood and tissue derived products devoid from tse-infectivity.

### Example 11

### Experimental Design Showing B-Cell Mediated Secondary Infection

As shown above, expression under the control of a human IRF1-promoter/Eμ-enhancer (“spleen mice”) results in high levels of PrP in the spleen, in particular in B and in T-cells (see example 10), but low levels in brain. In “spleen mice”, both at two weeks and at six months after i.p. inoculation with scrapie prions, high prion titers are found in spleen and thymus but not in brain, suggesting that the B and/or T-cells alone can sustain prion replication (see FIG. 5c). In order to study the interaction between the B-cells and the T-cells, PrP expression is targeted to a further cell type in PrP^{o/o} mice, namely to T-cells alone. Therefore, mice expressing PrP exclusively on T-cells (“T-cell mice”) are generated. Further, as a control experiment, in order assess whether (enhanced) PrP expression alone suffices to enable prion replication, PrP knock out mice expressing PrP exclusively in liver (“liver mice”) are created.

### Results

### Generation of Prnp^{o/o} Mice Transgenic for PrP Genes Controlled by Alien Promoters

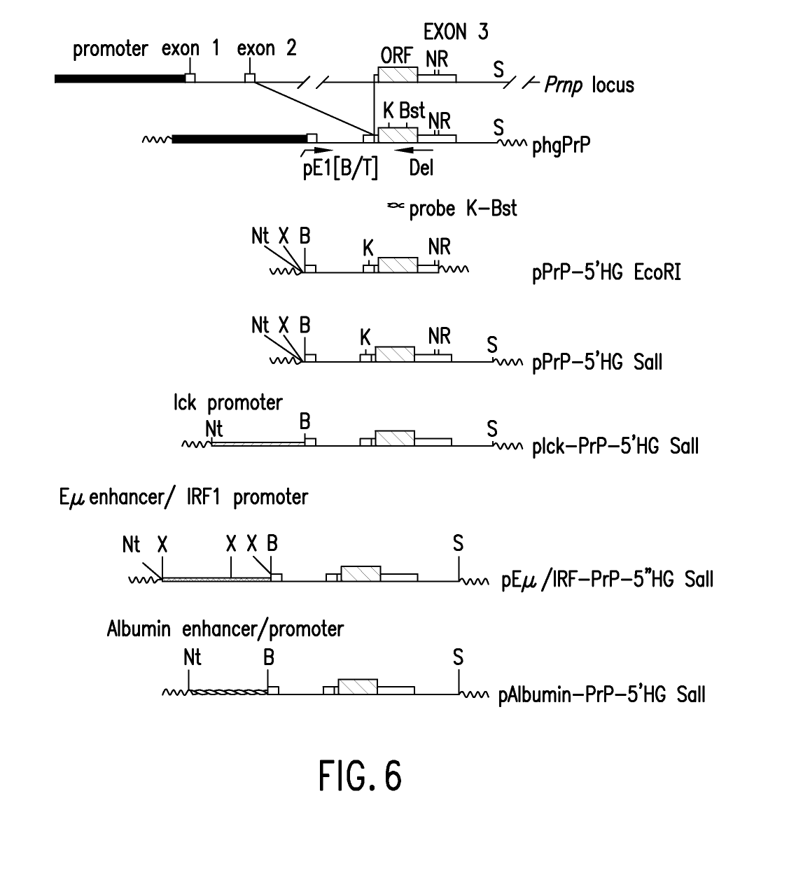

Introduction into Prnp^{o/o} mice of a half-genomic’ PrP transgene, which lacks the 12-kb intron, restored susceptibility to scrapie and the ability to replicate prions (Fischer, M., Rülicke, T., Raeber, A., Saller, A., Moser, M., Oesch, B., Brandner, S., Aguzzi, A. and Weissmann, C. (1996), Prion protein (PrP) with amino-proximal deletions restoring susceptibility of PrP knockout mice to scrapie. EMBO J. 15, 1255-1264). The inventors generated a promoterless PrP vector based on the ‘half-genomic’ PrP construct by introducing a BamHI site at the 5′ end of exon 1 into which cell and tissue-specific regulatory elements controlling the transcription of PrP were inserted (FIG. 6). Constructs were introduced into Prnp^{o/o} zygotes by pronuclear injection.

Mice Overexpressing PrP Under the Control of the IRF1-Promoter/Immunoglobulin Heavy Chain Enhancer (“Spleen Mice”).

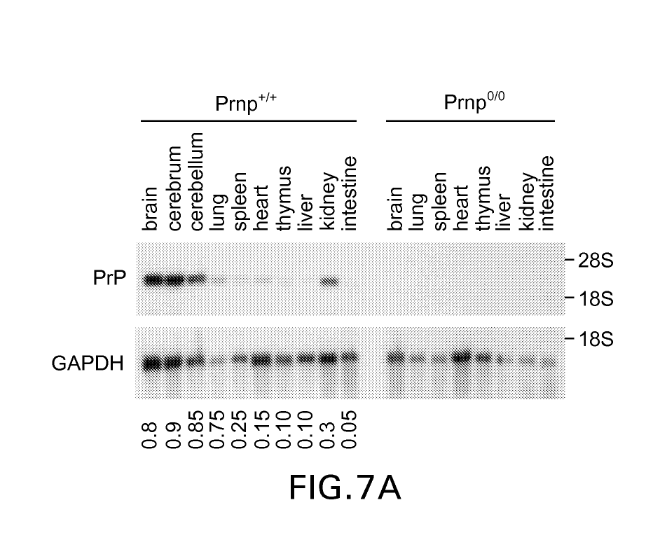

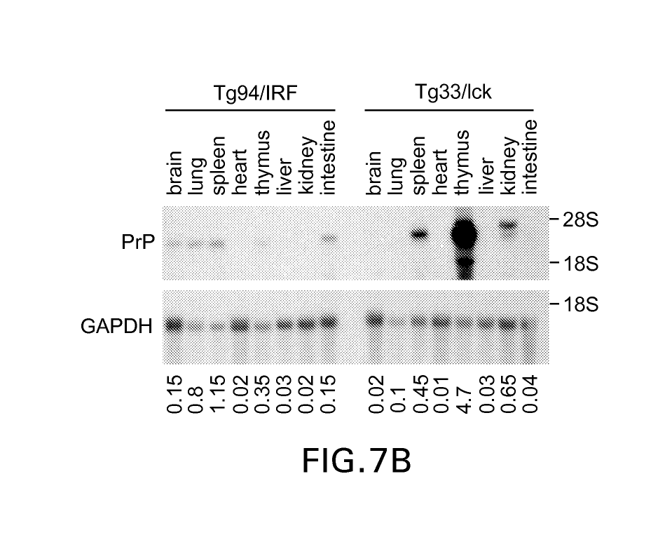

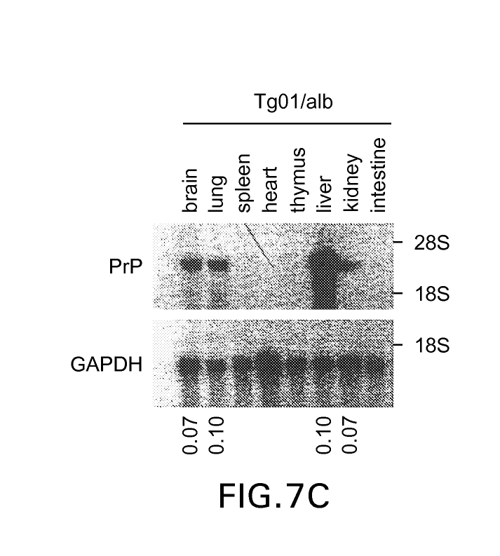

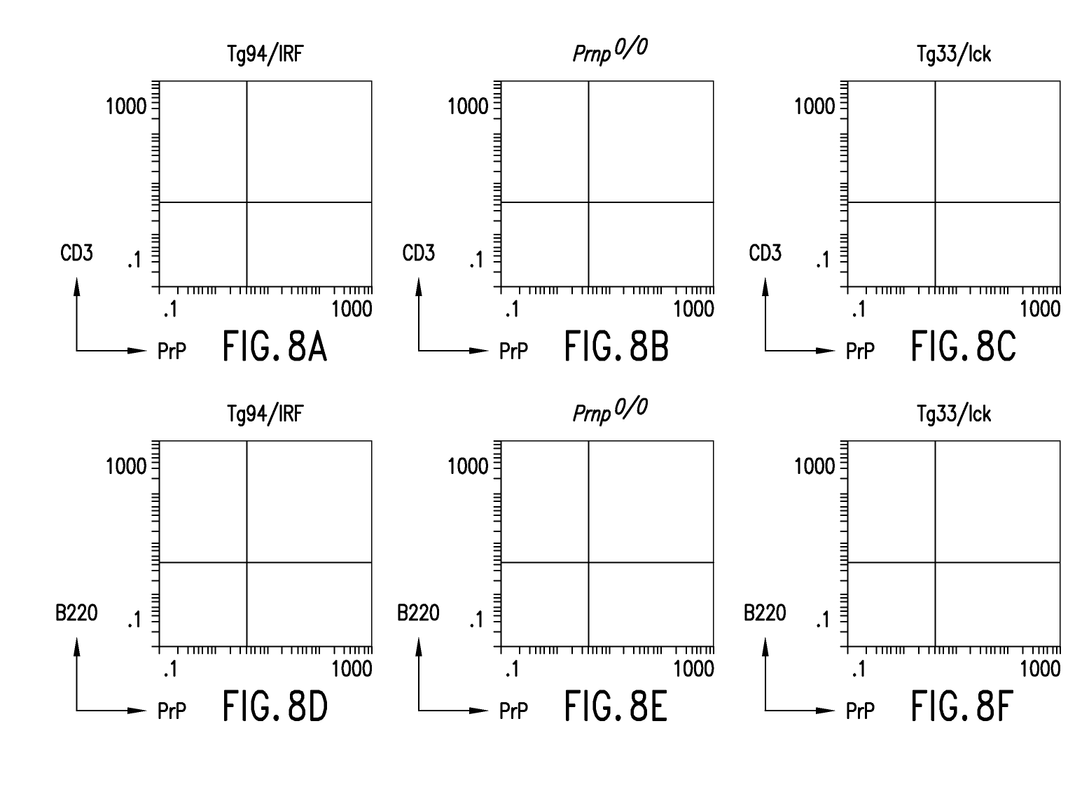

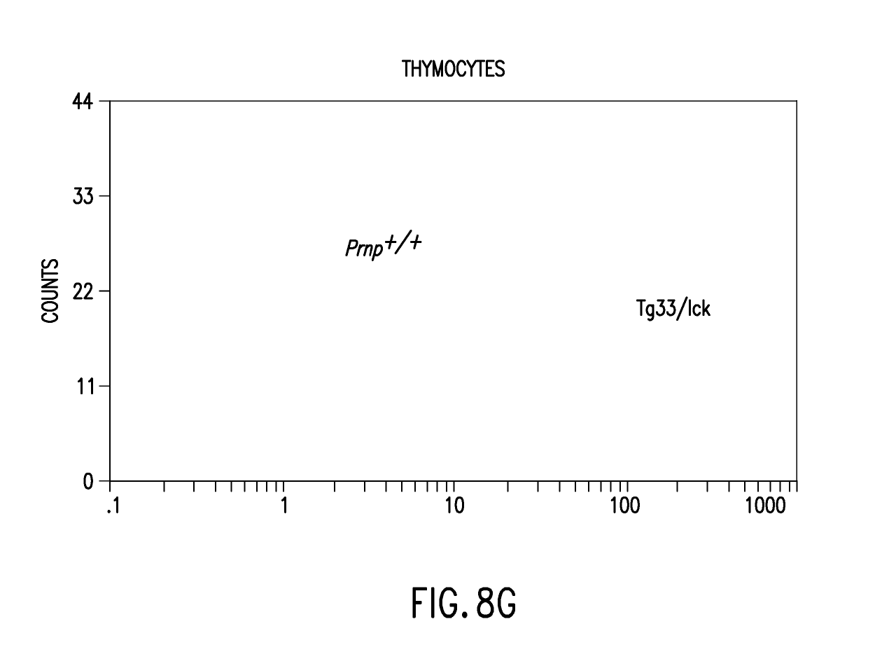

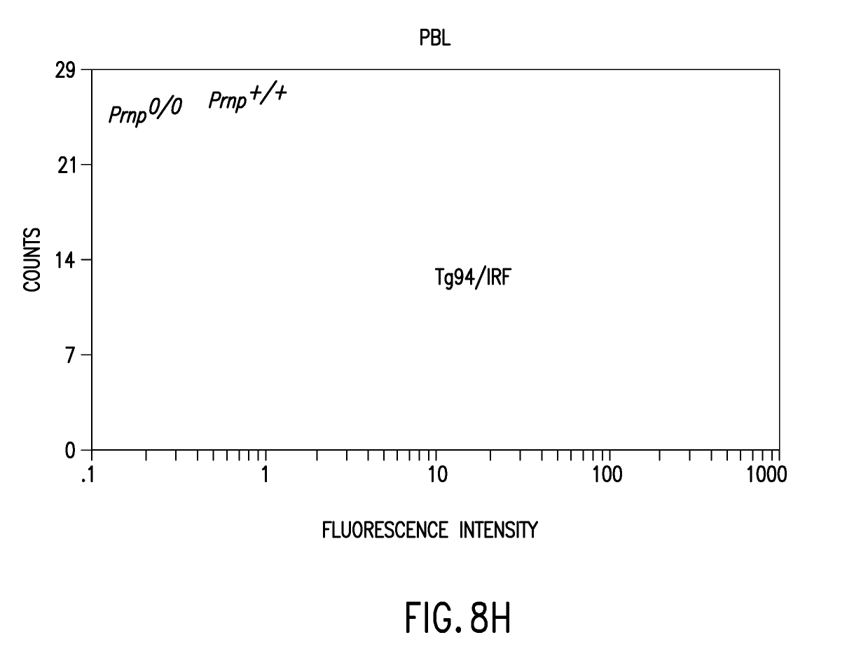

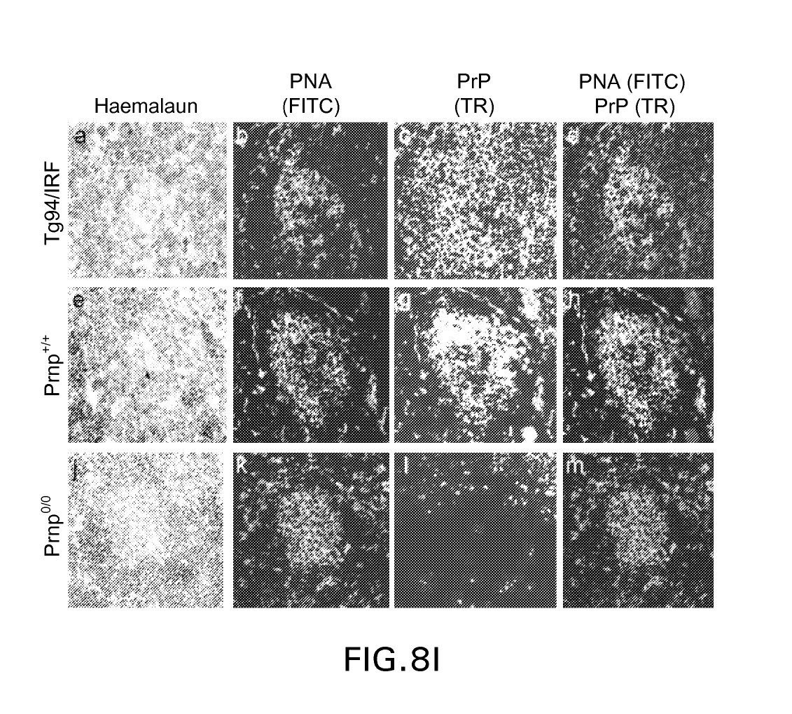

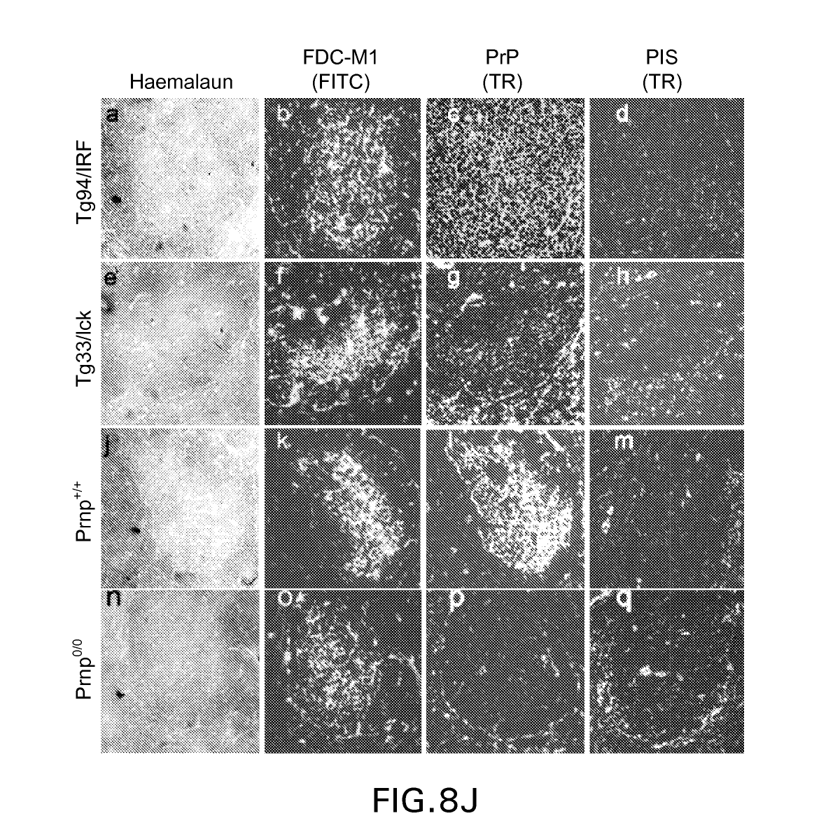

Two transgenic Prnp^{o/o} mouse lines carrying this construct, Tg94/IRF and Tg90/IRF, were established, with transgene copy numbers of 6 and 4, respectively. PrP mRNA levels in Tg94/IRF spleen and thymus were about 5 and 3 times higher, respectively, than in their wild-type counterparts (FIG. 7) but surprisingly PrP in spleen was >1000 times higher and in thymus >100 times higher than in wild-type (Table 8). PrP on the surface of peripheral blood leukocytes, as determined by cytofluorometry (FACS), was about 10-fold higher in Tg94/IRF than in wild-type mice (FIG. 8C). High levels of PrP were also observed on B and T lymphocytes of Tg94/IRF splenocytes (FIG. 8A). PrP in brain was 0.05 of that in wild-type (Table 8).

Cryosections of spleen from non-infected wild-type, Prnp^{o/o} and Tg94 mice were doubly stained for germinal center B cells (with peanut agglutinin (Kraal, G., Weissmann, I. L. and Butcher, e. C. (1982). Germinal centre B cells:’ antigen specificity and changes in heavy chain class expression. Nature 298, 377-9), green) and PrP (with PrP antiserum 340, red). In wild-type spleens, PrP was mainly present in germinal centers while in Tg94/IRF spleens it was uniformly distributed over white and red pulp (FIG. 8D). In FIG. 8E consecutive spleen sections were labeled with the FDC-specific antibody M1 (green) and PrP antiserum (red; simultaneous staining did not succeed), again revealing a striking overlap of FDC and PrP staining within germinal centers in wild-type spleens. In Tg94/IRF spleens, FDCs were stained in the germinal centers while PrP-specific fluorescence was uniform over the whole section, compatible with the FACS analysis which showed that B and T lymphocytes expressed PrP and with the assumption that also FDCs expressed PrP. However, the inventors did not ascertain coexpression of PrP and the FDC marker Ml.

Transgenic, wild-type (129/Sv-C57BL/6) and Prnp^{o/o} mice were inoculated intraperitoneally (i.p.) with 10⁶ LD₅₀ units of the RML isolate of mouse prions. As shown in Table 8, all wild-type mice developed scrapie after 194±5 days and died after 205±9 days, whereas all Prnp^{o/o} mice remained healthy for more than 500 days. All of 7 Tg94/IRF mice hemizygous for the transgene cluster developed scrapie symptoms after 452±15 days and died after 507±27 days, presumably because they expressed PrP in the brain, albeit at low levels (data not shown). When rendered homozygous for the transgene cluster, Tg94/IRF mice became ill at 268±24 days after inoculation and died of scrapie after 281±26 days (Table 8).

TABLE 8
Characteristics of transgenic mouse lines

| | PrP-encoding | Gene | PrP RNA | | PrP protein | | | Days to | Days to | |
|---|---|---|---|---|---|---|---|---|---|---|
| Line | gene | copy No.^{a} | (organ)^{b} | | (organ)^{c} | | Inoculum^{d} | symptoms | death | Animals^{e} |
| Prnp^{+/+} | Prnp | 2 | 1 | (brain) | 1 | (brain) | RML | 194 ± 5 | 205 ± 9 | 14/14 |
| | | | 1 | (spleen) | 1 | (spleen) | | | | |
| | | | 1 | (thymus) | 1 | (thymus) | | | | |
| Prnp^{0/0} | — | — | 0 | | 0 | | RML | >500 | | 0/10 |
| Tg94/IRF | Eμ/IRF1-PrP | 6 | 5 | (spleen) | >1000 | (spleen) | RML | 268 ± 24 | 281 ± 26 | 18/18 |
| | | | 0.2 | (brain) | <0.05 | (brain) | | | | |
| | | | 3 | (thymus) | >100 | (thymus) | | | | |
| Tg90/IRF | Eμ/IRF1-PrP | 4 | nd | | nd | | | | | |
| Tg33/Ick | Ick-PrP | 20 | 2 | (spleen) | 40 | (spleen) | RML | >500 | | 0/6 |
| | | | 0.025 | (brain) | <0.001 | (brain) | | | | |
| | | | 40 | (thymus) | >100 | (thymus) | | | | |
| Tg71/Ick | Ick-PrP | 10 | nd | | nd | | | | | |
| Tg01/alb | albumin-PrP | 20 | 11 | (liver) | 5 | (liver) | RML | >400 | | 0/6 |
| | | | 0.09 | (brain) | 0.1 | (brain) | | | | |

| | | |
|---|---|---|
| 0 | (spleen) | nd |
| 0 | (thymus) | nd |

| | | | | |
|---|---|---|---|---|
| Tg19/alb | albumin-PrP | 2 | nd | nd |

^{a}Relative to wild-type; determined by Southern blot analysis. All transgenic animals were homozygous for the transgene and all mice had a mixed 129Sv/C57BI background.
^{b}Relative to the corresponding wild-type organ; determined by quantitative Northern blot analysis.
^{c}Relative to the corresponding wild-type organ; determined by densitometric analysis of Western blots.
^{d}All animals were inoculated intraperitoneally with_100 μl of a 1% (w/v) brain homogenate (RML isolate)
^{e}The number of mice that developed clinical signs of scrapie divided by the total number of mice inoculated
nd: not determined

Wild-type and Tg94/IRF mice hemizygous for the transgene cluster were inoculated i.p. As shown in Table 9, two weeks after inoculation spleen extracts from Tg94/IRF mice and wild-type animals had the same titer, about 7 logLD₅₀ units/ml 10% homogenate and no infectivity was detected in brain. Six months after inoculation the titers of Tg94/IRF spleen extracts were essentially unchanged, somewhat higher than the value of 6.5 for wild-type spleen and no infectivity was detected in. Tg94/IRF brains, as compared to 8 logLD₅₀ units/ml 10% homogenate for wild-type. However, one year after inoculation, extracts from hemizygous_Tg94/IRF thymus, spleen and brain showed prion titers of about 5.5, 5, and 7 log LD₅₀ units/ml 10% homogenate, respectively (FIG. 5c). The late appearance of prions in brain can be attributed to low levels of PrP expression in TG94/IRF brains as compared to wild-type mice (Büeler, H., Raeber, A., Saller, A., Fischer, M., Aguzzi, A. and Weissmann, C. (1994). High prion and PrPSc levels but delayed onset of disease in scrapie-inoculated mice heterozygous for a disrupted PrP gene. Molecular Medicine 1, 19-30).
Mice Overexpressing PrP on T Lymphocytes Under the Control of the Lck Promoter (“T-Cell Mice”).

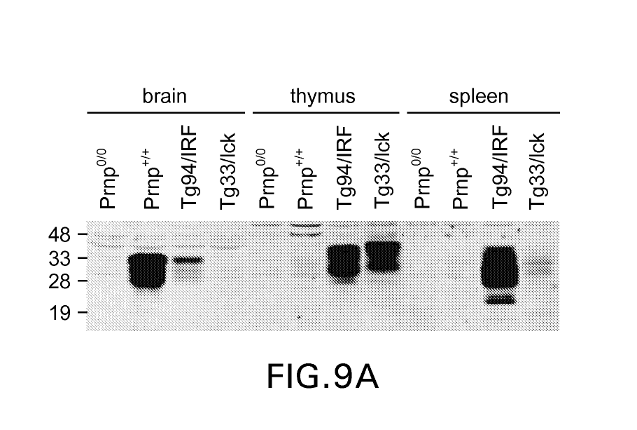

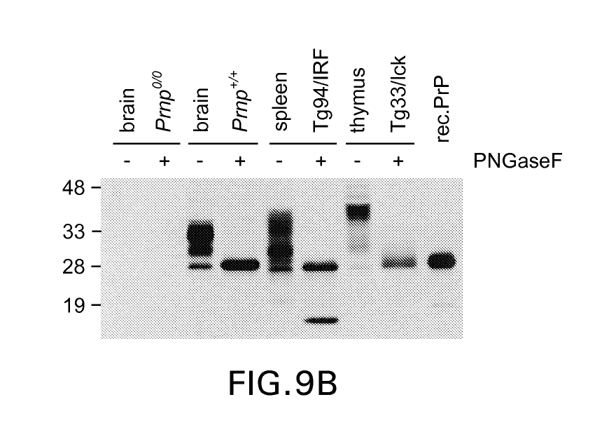

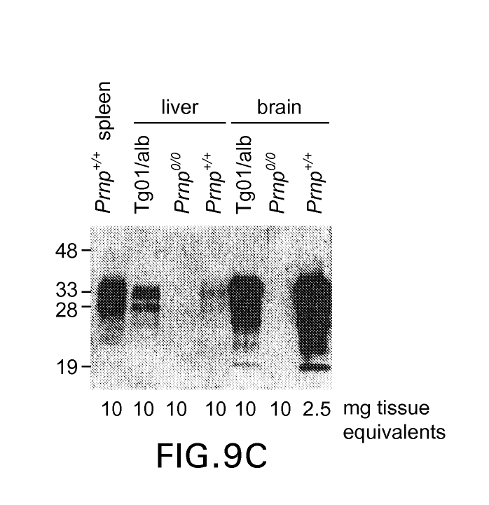

Transgenic mouse lines with ectopic PrP expression were generated with the T-lymphocyte-specific Lck promotor (Chaffin, K. E., Beals, C. R., Wilkie, T. M., Forbush, K. A., Simon, M. I. and Perlmutter, R. M. (1990). Dissection of thymocyte signaling pathways by in vivo expression of pertussis toxin ADP-ribosyltransferase. EMBO J. 9, 3821-3829). Two lines, Tg33/Ick and Tg71/Ick, which harbored 20 and 10 copies of the transgene, respectively, were studied. Northern blot analysis (FIG. 7) revealed PrP transcript levels in the thymus at least 50-fold higher than in wild-type. Significant levels of PrP mRNA were also found in spleen and kidney. A PrP RNA species longer than the major transcript seen in thymus and spleen was observed in Tg33/Ick kidney, reflecting perhaps a splicing variant or the use of a conjectural further-downstream polyadenylation site. Low levels of PrP transcripts were detected in brain, lung and intestine only upon longer exposure of the Northern blot (not shown). Tg33/Ick thymus and spleen had PrP levels that were at least 100-fold and 40-fold higher, respectively, than in wild-type. PrP was undetectable in Tg33/Ick brain (FIG. 9A). The high level of PrP expression on T lymphocytes was confirmed by FACS analysis of Tg33/Ick thymocytes (FIG. 8B) and estimated to be 50-fold higher than in wild-type. No PrP expression was detected in Tg33/Ick splenic B lymphocytes whereas splenic T lymphocytes were strongly positive for PrP (FIG. 8A). Immunohistochemical analysis of Tg33/Ick spleens (FIG. 8E) showed that PrP expression (red) was predominantly in the perifollicular T cell area while, the germinal centers, where the FDCs (green) were located, showed little red fluorescence over background.

PrP from Tg33/Ick thymus had a distinctly lower electrophoretic mobility on SDS-polyacrylamide gels than that of Prnp^{+/+} brain (FIG. 9A). Much of the heterogeneity of PrP molecules is attributed to various degrees of N-linked glycosylation on asparagine 181 and 197 (DeArmond, S. J., Sanchez, H., Yehiely, F., Qiu, Y., Ninchak-Casey, A., Daggett, V., Camerino, A. P., Cayetano, J., Rogers, M., Groth, D., Torchia, M., Tremblay, P., Scott, M. F., Cohen, F. E. and Prusiner, S. B. (1997). Selective neuronal targeting in prion disease. Neuron 19, 1337-48). After deglycosylation with PNGase, F, PrP from both spleen and thymus of Tg33/Ick mice was reduced to a single PrP species with about the same mobility as recombinant PrP from E. coli, i.e. an apparent molecular weight of about 27 kDa (FIG. 9B). This confirmed that PrP undergoes organ and/or cell-specific glycosylation.

To determine whether PrP^{c} expression in T lymphocytes of Tg33/Ick mice enabled prion replication in thymus and spleen, the inventors assayed tissue extracts pooled from two animals sacrificed at 2 weeks, 6 months and 12 months after i.p. inoculation.

In the case of scrapie-infected Tg33/Ick mice, homogenates prepared from spleen two weeks after inoculation led to disease in two out of four indicator CD-1 mice after 192+39 days while samples from thymus extracts produced disease in one out of four CD-1 mice after 181 days. No infectivity was detected in Tg33/Ick spleen, thymus or brain or 6 or 12 months after inoculation, except for a spleen extract collected 1 year after inoculation which led to scrapie in one of four CD-1 mice (Table 9). Thymus and liver homogenates from Prnp^{o/o} mice also occasionally led to disease in one or two of four indicator mice. Most’ likely, these borderline infectivities are due to prions persisting from the inoculum and thus do not appear in FIG. 5b, which displays the overall results of the Tg33/Ick mice study (Sailer, A. Büeler, H., Fischer, M., Aguzzi, A. and Weissmann, C. (1994). No propagation of prions in mice devoid of PrP. Cell 77, 967-968). Six months after i.p. inoculation wild-type mice had titers of about 6.5, 4.5 and 8 logLD₅₀ units/ml 10% homogenate in spleen, thymus and brain, respectively (see FIG. 5a).

Thus, it has been shown that even vast overexpression of PrP^{c} on T-cells, comparable to levels found in wild-type brain, is not sufficient to allow prion replication in thymus or spleen of Prnp^{o/o} mice, if PrP-expressing B-cells are absent. Thus, it appears that tse-infected B-cells are mandatorily required for prion replication in T-cells.

TABLE 9
Infectivity bioassay of organs from RML-inoculated mice

| | Time p.l. | | | Illness | | Death | | Titer ^{c} |
|---|---|---|---|---|---|---|---|---|
| Donor | (days) | Organ ^{a} | Recipient | (days) | (n/nt)^{b} | (days) | (n/nt)^{b} | log LD50/ml 10% |
| Prnp ^{+/+} | 14 | brain | CD-1 | >300 | 0/4 | | | |
| | 14 | spleen | CD-1 | 138 ± 3 | 4/4 | 163 ± 4 | 4/4 | 7 |
| | 14 | thymus | CD-1 | 177 ± 19 | 4/4 | 200 ± 24 | 4/4 | 35 |
| | 14 | liver | CD-1 | 247 | 1/4 | 250 | 1/4 | |
| | 180 | brain | CD-1 | 132 ± 4 | 4/4 | 150 ± 4 | 4/4 | 8 |
| | 180 | spleen | CD-1 | 159 ± 3 | 4/4 | 168 ± 5 | 4/4 | 65 |
| | 180 | thymus | CD-1 | 183 ± 22 | 4/4 | 189 ± 23 | 4/4 | 45 |
| | 180 | liver | CD-1 | >300 | 0/4 | | | |
| Prnp ^{0/0} | 14 | brain | CD-1 | >300 | 0/4 | | | |
| | 14 | spleen | CD-1 | >300 | 0/3^{d} | | | |
| | 14 | thymus | CD-1 | 289 | 1/4 | 295 | 1/4 | −1 |
| | 14 | liver | CD-1 | 244 ± 54 | 2/4 | 247 ± 52 | 2/4 | −15 |
| | 180 | brain | CD-1 | >300 | 0/4 | | | |
| | 180 | spleen | CD-1 | >300 | 0/4 | | | |
| | 180 | thymus | CD-1 | >300 | 0/4 | | | |
| | 180 | liver | CD-1 | 210 | 1/4 | 240 | 1/4 | −1 |
| | 365 | brain | CD-1 | >300 | 0/4 | | | |
| | 365 | spleen | CD-1 | >300 | 0/4 | | | |
| | 365 | thymus | CD-1 | >300 | 0/3^{d} | | | |
| | 365 | liver | CD-1 | >300 | 0/3^{d} | | | |
| Tg33/Ick (hemi) | 14 | brain | CD-1 | >300 | 0/4 | | | |
| | 14 | spleen | CD-1 | 192 ± 39 | 2/4 | 195 ± 41 | 2/4 | −15 |
| | 14 | thymus | CD-1 | 181 | 1/4 | 189 | 1/4 | −1 |
| | 180 | brain | CD-1 | >300 | 0/4 | | | |
| | 180 | spleen | CD-1 | >300 | 0/4 | | | |
| | 180 | thymus | CD-1 | >300 | 0/3 | | | |
| | 365 | brain | CD-1 | >300 | 0/4 | | | |
| | 365 | spleen | CD-1 | 222 | 1/4 | 222 | 1/4 | −1 |
| | 365 | thymus | CD-1 | >300 | 0/4 | | | |
| Tg01/alb (hemi) | 14 | brain | CD-1 | >300 | 0/4 | | | |
| | 14 | spleen | CD-1 | >300 | 0/4 | | | |
| | 14 | liver | CD-1 | >300 | 0/4 | | | |
| | 180 | brain | CD-1 | >300 | 0/4 | | | |
| | 180 | spleen | CD-1 | >300 | 0/4 | | | |
| | 180 | liver | CD-1 | >300 | 0/4 | | | |
| | 365 | brain | CD-1 | >300 | 0/4 | | | |
| | 365 | spleen | CD-1 | >300 | 0/4 | | | |
| | 365 | liver | CD-1 | >300 | 0/4 | | | |
| Tg94/IRF (hemi) | 14 | brain | CD-1 | >300 | 0/4 | | | |
| | 14 | spleen | CD-1 | 152 ± 10 | 4/4 | 163 ± 12 | 4/4 | 7 |
| | 180 | brain | CD-1 | >300 | 0/4 | | | |
| | 180 | spleen | CD-1 | 148 ± 13 | 4/4 | 160 ± 6 | 4/4 | 7 |
| | 365 | brain | Tg20 | 64 ± 2 | 4/4 | 66 ± 2 | 4/4 | 7 |
| | 365 | spleen | Tg20 | 82 ± 5 | 4/4 | 86 ± 5 | 4/4 | 5 |
| | 365 | thymus | Tg20 | 78 ± 7 | 4/4 | 81 ± 8 | 4/4 | 55 |

^{a} Organs were prepared as 10% homogenates in 0.32 M sucrose. Thirty μl of a 10-fold dilution (1% homogenate) in PBS-5% BSA were inoculated intracerebrally into recipient mice as indicated.
^{b}The number of mice with scrapie divided by the total number of mice inoculated
^{c} Titers were determined by the incubation time method using standard curves for CD-1 mice (Büeler et al., 1903) or Tg20 mice (Brandner el al, 1996), as indicated. Limit of detection, about 1 log LD50/ml 10% homogenate
^{d}Mouse died of intercurrent disease.
(hemi) = hemizygous for the transgene

Mice Overexpressing PrP Under the Control of the Albumin Promoter

Transgenic mice with ectopic expression of PrP in the liver (“liver mice”) were generated with use of the albumin enhancer/promoter which was reported to direct efficient, liver specific expression in transgenic mice (Pinkert, C, Ornitz, D. M., Brinster, R. L. and Palmiter, R. D. (1987). An Albumin enhancer located 10 kb upstream functions along with its promoter to direct efficient, liver-specific expression in transgenic mice. Genes Dev. 1, 268-276). two lines of transgenic mice, Tg01/alb and Tg19/alb, harbored 20 and 2 copies, respectively, of the hybrid transgene. Northern blot analysis of Tg01/alb tissues revealed highest levels of Prp mRNA in the liver and low levels in lung, brain and kidney (FIG. 7). To determine PrP expression, the inventors immunoprecipitated PrP from extracts of 10 mg liver and brain and displayed it by immunoblot analysis (FIG. 9C). PrP levels in Tg 01/alb liver were at least 5-fold higher than those in wild-type liver, but still about 2-3 times lower than in wild-type spleen. PrP levels in Tg01/alb brain were unexpectedly high, about 10% of those in PrnP^{+/+} brain. None of the Tg01/alb mice developed scrapie disease within 400d of i.p. inoculation (table 8) or within 300 days of i.c. inoculation. Tissues from i.p. inoculated Tg01/alb mice were bio-assayed for infectivity (table 9). No infectivity was, detected in liver, brain and spleen of Tg01/alb mice at any time after inoculation.

Thus, overexpression of PrP in the liver of Prnp^{o/o} mice, under the control of the albumin promoter, failed to sustain prion replication in liver, spleen or brain. These results show that PrP^{c} overexpression alone is not sufficient to allow prion replication in any tissue.

The Fate of the Inoculum.

Although high prion titers are found in spleen within few days after i.c. or i.p. inoculation, it is in principle not immediately clear whether this reflects de novo synthesis in the LRS or scavenging of infectious agent generated in brain or derived from the inoculum. Inoculation with very low prion doses had shown that net increase of infectious agent resulted in the spleen (Clarke and Haig, 1971), however, for the sake of absolute scrutiny, it could not be excluded that the agent was being synthesized in the brain and transported to the LRS. To resolve this question, the inventors inoculated Tg94/IRF mice i.p. with a very low dose of RML prions (3.5 log LD₅₀ i.c. units) and analyzed spleen homogenates at various times after injection by endpoint titration. As shown in table 10, prion titers in the spleen (in logLD₅₀ i.c. units/ml 10% homogenate) rose from 2 at two weeks after inoculation to about 6 after 4 weeks and remained at this level up to 12 weeks. Because a spleen weighs about 100 mg, this represents an increase of at least 2.5 logs over input, showing that prions are replicated in the spleen of i.p. inoculated Tg94/IRF mice and are not due to residual inoculum or import from the brain, which even at 6 months contains no detectable infectivity.

TABLE 10
Titration of scrapie infectivity in spleens of
Tg94/IRF mice inoculated intraperitoneally^{a}

| | Log Dilution^{b} | | | | | | |
|---|---|---|---|---|---|---|---|
| Time after i.p. | 0 | −1 | −2 | −3 | −4 | −5 | |
| inoculation (weeks) | days to disease in indicator mice (n/nₒ) | | | | | | Titer^{c} |
| 2 | 73 +/− 1 | — | nd | nd | nd | nd | 2 |
| | (4/4) | (0/4) | | | | | |
| 4 | nd | 79 ± 5 | 84 ± 6 | 104 ± 2 | 121 ± 4 | — | 6 |
| | | (4/4) | (2/2) | (3/4) | (3/4) | (0/4) | |
| 8 | nd | 78 ± 4 | nd | 103 ± 13 | 110 | 118 | 5 |
| | | (4/4) | | (4/4) | (1/4) | (1/4) | |
| 12 | nd | 71 ± 0 | nd | 94 ± 7 | 111 ± 11 | — | 6 |
| | | (4/4) | | (4/4) | (4/4) | (0/4) | |

^{a}Homozygous Tg94/IRF mice were inoculated i.p. with 3.5 logLD₅₀ i.c. units of RML prions. At the times indicated, mice were killed and the titer in the spleen was determined by endpoint titration in Tg20 mice. The numbers in the Table indicate the time elapsed (in days) to appearance of symptoms and the fraction (n/nₒ) of mice falling sick, n.d., not done.
^{b}Serial 10-fold dilutions of 10% spleen homogenates were prepared in PBS-5% BSA and 30 μl were inoculated intracerebrally into Tg20 indicator mice.
^{c}LogLD₅₀ units/ml 10% homogenate. Calculated by multiplying the LD₅₀ units at the endpoint dilution with (33 x dilution factor). Endpoints were calculated according to Reed and Muench (1938). The American Journal of Hygiene, Vol. 27. No. 3.

### 11.1. DNA Constructions

DNA constructions (FIG. 6) were carried out according to standard cloning protocols (Ausubel, F. M., Brent, R., Kingston, R. E., Moore, D. D., Seldman, J. G., Smith, J. A. and Struhl, K. (1987) Current protocols in molecular biology. John Wiley & Sons, New York; Sambrook, J., Fritsch, E. F. and Maniatis, T. (1989). Molecular Cloning: A Laboratory Manual. Cold Spring Harbor Laboratory Press, Cold Spring Harbor, N.Y.). The ‘half-genomic’ PrP vector (phgPrP), pPrPcDNA and pPrPEl HE23R1 have been described (Fischer, M., Rülicke, T., Raeber, A., Saller, A., Moser, M., Oesch, B., Brandner, S., Aguzzi, A. and Weissmann, C. (1996). Prion protein (PrP.) with amino-proximal deletions restoring susceptibility of PrP knockout mice to scrapie. EMBO J. 15, 1255-1264). The latter two constructs, but not phgPrP, have a G->A point mutation in the 5′ non-coding region, at position 25 of exon 1 (underlined) in the pPrPcDNA: GTC-GGA-TCC-GCA-GAC-CGA-TTC-TGG-ACG (SEQ ID NO:1). Plasmids encoding a promoterless ‘half-genomic’ PrP vector and the tissue-specific expression constructs were generated as follows. pPrP-5′HG Sall: A 2.7-kb PCR product was prepared using phgPrP as the template, the 5′ terminal primer pEl [B/T] (5′)tgtcggatccagcagaccgattctgg(3′) (SEQ ID NO:2) to introduce a unique BamHI site (underlined) 5′ of exon 1 and the 3′ terminal primer (Del) 5′tccccagcatgtagccaccaagg(3′) (SEQ ID NO:3). The 2.3-kb BamHI-Kpnl fragment of this PCR product and the 1.3-kb fragment obtained from pPrPE1i1E23R1 by partial digestion with Kpnl and EcoRI (comprising exon 2 and part of exon 3 with the entire coding region) were joined to BamHI and EcoRI-restricted and dephosphorylated pBluescript (Stratagene) in a three-way ligation. The resulting plasmid pPrP-5′HG EcoRI contained the half-genomic promoterless PrP gene extending up to the EcoRI site in the 3′ untranslated region of Prnp. Plasmid pPrP-5′HG EcoRI was digested with Sall (within the pBluescript polylinker) and Narl and joined to the 3-kb Narl-Sall fragment from phgPrP (comprising the 3′ end of Prnp). plck-PrP-5 ′HG Sall: The Lck proximal promoter expression cassette in plasmid P1017 (Chaffin, K. E., Beals, C. R., Wilkie, T. M., Forbush, K. A., Simon, M. I. and Perlmutter, R. M. (1990). Dissection of thymocyte signaling pathways by in vivo expression of pertussis toxin ADP-ribosyltransferase. EMBO J. 9, 3821-3829) was excised as a 3.1-kb BamHI-Notl fragment and cloned into the Notl and BamHI-cleaved pPrP-5′HG Sall. pEμ/IRFl-PrP-5′HG Sall: The human interferon regulatory factor 1 (IRF1) promoter sequence was amplified by PCR using plasmid p-4921IRF1cat (Harada, H., Takahashi, E., Itoh, S., Harada, K., Hori, T. A. and Taniguchi, T. (1994). Structure and regulation of the human interferon regulatory factor 1 (IRF-1) and IRF-2 genes: implications for a gene network in the interferon system. Mol. Cell. Biol. 14, 1500-9) as the template, the 5′ terminal primer (IRFtop: 5′-tttctagaggagccaggctgc-3′; SEQ ID NO:4) containing an artificial Xbal site (underlined) and the 3′ terminal primer (IRFbottom: 5′-agggatcctcgactaaggagtgg-3′; SEQ ID NO:5) containing an artificial BamHI site (underlined). The 560-bp Xbal-BamHl fragment of this PCR product and the 6-kb BamHI-Sall fragment from pPrP-5′HG Sall were joined to the 3-kb XbaI-Sall fragment of pPrP-5′HG Sall in a three-way ligation. The resulting plasmid pIRF1-PrP-5′HG Sall was linearized by partial digestion with Xbal and joined to a 2.1-kb Xbal vector fragment, containing the Eμ immunoglobulin heavy chain enhancer from pEμ-myc (Hayday, A. C, Gillies, S. D., Saito, H., Wood, C, Wiman, K., Hayward, W. S, and Tonegawa, S. (1984). Activation of a translocated human c-myc gene by an enhancer in the immunoglobulin heavy-chain locus. Nature 307 334-340). pAlbumin-PrP-5′HG Sall: The albumin promoter/enhancer was excised from plasmid 2335A-1 (equivalent to the construct NB (Pinkert, C. A., Ornitz, D. M., Brinster, R. L. and Palmiter, R. D. (1987). An albumin enhancer located 10 kb upstream functions along with ist promoter to direct efficient, liver-specific expression in transgenic mice. Genes Dev. 1, 268-276) as a 2.0-kb BamHI-Notl fragment and joined to the Notl and BamHI-restricted and dephosphorylated pPrP-5′HG Sall.

### 11.2. Generation of Transgenic Mice

Plasmid DNA was digested with Notl and Sall and prepared for microinjection as described previously (Fischer, M., Rülicke, T., Raeber, A., Saller, A., Moser, M., Oesch, B., Brandner, S., Aguzzi, A. and Weissmann, C. (1996). Prion protein (PrP) with amino-proximal deletions restoring susceptibility of PrP knockout mice to scrapie. EMBO J. 15, 1255-1264). Microinjection into the male pronucleus of homozygous Prnp^{o/o} zygotes and re-implantation were as described (Brinster, R. L., Chen, H. Y., Trumbauer, M. E., Yagle, M. K. and Palmiter, A. D. (1985). Factors affecting the efficiency of introducing foreign DNA into mice by microinjecting eggs. Proc. Natl. Acad. Sci. USA 82, 4438-4442; Hogan, B., Beddington, R., Costantini, F. and Lacy, E. (1994) Manipulating the mouse embryo. A laboratory manual, CSHL Press, New York). Founders were identified by Southern analysis of Pstl-digested tail DNA using a mouse PrP GRF probe (probe A in (Büeler, H., Fischer, M., Lang, Y., Bluethmann, H., Lipp, H.-P., DeArmond, S. J., Prusiner, S. B., Auet, M. and Weissmann, C. (1992). Normal development and behaviour of mice lacking the neuronal cell-surface PrP protein. Nature 356, 577-582)). Transgene-positive founders were mated to Prnp^{o/o} mice and lines were established from F1 progeny. Transgene copy numbers were estimated relative to Prnp^{o} alleles on Southern blots using the phosphorimager and IMAGEQUANT™ software (Molecular Dynamics, USA). Alternatively, Prnp^{o} alleles and Prnp-transgenes were detected by PCR as detailed earlier (Fischer/M., Rülicke, T., Raeber, A., Saller, A., Moser, M., Oesch, B., Brandner, S., Aguzzi, A. and Weissmann, C. (1996). Prion protein (PrP) with amino-proximal deletions restoring susceptibility of PrP knockout mice to scrapie. EMBO J. 15, 1255-1264). Seven transgenic mouse lines were established with the pAlbumin-PrP-5′HG Sall construct and two lines with the highest expression of PrP mRNA in liver, designated Prnp^{o/o}TgN(albPrnp)181Zbz (Tg01/alb) and Prnp^{o/o}TgN(albPrnp)185Zbz(Tg19/alb), were chosen for further studies. Five transgenic lines were generated with the plck-PrP-5′HG Sall construct. The two lines Prnp^{o/o}TgN(IckPrnp)192Zbz (Tg33/Ick) and Prnp^{o/o}TgN(IckPrnp)193Zbz (Tg71/Ick) with highest PrP expression in the thymus were further analyzed. Two of 3 transgenic lines containing the pEμ/IRF1-PrP-5′HG Sall construct with high PrP expression levels in the spleen, Prnp^{o/o}TgN(IRFlPrnp)196Zbz (Tg9 4/IRF) and Prnp^{o/o}TgN(IRFlPrnp)198Zbz (Tg90/IRF) were maintained.

### 11.3. Northern Analysis

Total RNA from organs was prepared using the RNeasy RNA extraction kit (Qiagen). Aliquots (10 μg) of total RNA were run on 1% formaldehyde-agarose gels and blotted onto HYBOND™-N+ (Amersham) membranes in 20×SSC. Prehybridization and hybridization were performed with Quickhyb (Stratagene) according to the manufacturer's instructions. Probes, ³²P-labeled by the random primer method (Prime-It, Stratagene), were the 256-bp Kpnl-BstEII fragment of the mouse PrP ORF (probe A, which corresponds to the PrP segment deleted in the Prnp^{o/o} mice (Büeler, H., Fischer, M., Lang, Y., Bluethmahn, H., Lipp, H.-P., De Armond, S. J., Prusiner, S. B., Aguet, M. and Weissmann, C. (1992). Normal development and behaviour of mice lacking the neuronal cell surface PrP protein. Nature 356, 577-582)) and the 490-bp Xholl fragment of rat glyceraldehyde-3-phosphate dehydrogenase (GAPDH) subcloned into pSP64 (Fort, P., Marty, L., Piechaczyk, M., el Sabrouty, S., Dani, C, Jeanteur, P. and Blanchard, J. M. (1985). Various rat adult tissues express only one major mRNA species from the glyceraldehyde-3-phosphate-dehydrogenase multigenic family. Nucleic Acids Res. 13, 1431-1442). Quantification was carried out with a phosphorimager and IMAGEQUANT™ software (Molecular Dynamics, USA).

### 11.4. Immunoprecipitation

Tissue homogenates (10% w/v) were prepared in Tris-buffered saline (TBS) (10 mM Tris-HCl (pH 8.0), 140 mM NaCl) containing 2% Sarkosyl and 1 mM phenylmethylsulfonyl fluoride. Insoluble material was removed by centrifugation at 2000×g for 15 min. For immunoprecipitation, aliquots of the supernatant were diluted 5-fold in TBS, precleared by centrifugation at 13,000×g for 15 min and’ incubated with excess Sepharose 4B-linked monoclonal antibody 6H4 (Korth, C, Stierli, B., Streit, P., Moser, M., Schaller, O., Fischer, R., Schulz-Schaeffer, W., Kretzschmar, H., Raeber, A., Braun, U., Ehrensperger, F., Hornemann, S., Glockshuber, R., Riek, R., Billeter, M., Wuthrich, K. and Oesch, B. (1997). Prion (PrPSc)-specific epitope defined by a monoclonal antibody. Nature 390, 74-7) for 2 h at 4° C. Sepharose beads were centrifuged at 13,000×g for 3 min and the pellet washed successively in TBS-0.2% Sarkosyl, in TBS-0.5 M NaCl-0.2% NP-40, TBS-0.5% NP-40 and finally in TBS for 5 min, all at room temperature. Pellets were boiled in SDS-sample buffer and analyzed by immunoblotting.

### 11.5. Immunoblot Analysis

Tissue homogenates were prepared and analyzed as described previously (Raeber, A. J., Race, R. E., Br'andner, S., Priola, S. A., Saller, A., Bessen, R. A., Mucke, L., Manson, J., Aguzzi, A., Oldstone, M. B. A., Weissmann, C. and Chesebro, B. (1997). Astrocyte-specific expression of hamster prion protein (PrP) renders PrP knockout mice susceptible to hamster scrapie. EMBO J. 16, 6057-65). PNGaseF digestion of tissue homogenate samples (40 μg protein) was with 500 units of PNGaseF (NewEngland Biolabs, USA) for 2 h at 37° C. according to the manufacturer's instructions. PrP was detected with the polyclonal PrP antibody 1B3 (Farquhar, C. F., Somerville, R. A. and Ritchie, L. A. (1989). Postmortem immunodiagnosis of scrapie and bovine spongiform encephalopathy. J. Virol Methods 24, 215-221) diluted 1:10,000 and horseradish peroxidase-conjugated swine anti-rabbit immunoglobulins, diluted 1:5000 (DAKO, Glostrup, DK), developed using the enhanced ‘chemiluminescence kit (Amersham) and exposed to Kodak X-ray film. An appropriate exposure was scanned with a laser densitometer (Molecular Dynamics, USA) and quantified with IMAGEQUANT™ software.

### 11.6. Immunocytochemistry

Frozen sections (5-μm) from spleen were stained with acidic haemalaun. Immunofluorescence staining on consecutive cryosections and double-color immunofluorescence were performed with polyclonal_anti-PrP antiserum R340 (raised in rabbits using murine rPrP; 1:800 dilution) and biotinylated peanut agglutinin (1:400 dilution, Vector Laboratories, Burlingame, USA) or follicular dendritic cell marker FDC-M1 (clone 4C1 1, 1:300 dilution) on frozen acetone-fixed spleen sections. PrP and FDC were visualized by immunofluorescence using the Tyramide Signal Amplification kit (NEN Life Science Products, Brussels, Belgium) with TEXAS REDO-conjugated:avidin (1:100 dilution, Rockland, Gilbertsville, USA) and fluorescein isothiocyanate-conjugated streptavidin (1:100 dilution, Serotec, Oxford, UK). For controls, primary antibodies were omitted or pre-immune serum was used.

### 11.7. FACS Analysis

Single-cell suspensions from thymus and spleen were prepared in PBS with 2% fetal calf serum, 20 mM EDTA and 0.1% sodium azide (FACS buffer). Peripheral blood lymphocytes from heparinized blood were enriched by lysis of erythrocytes. For flow cytometric analysis (EPICS XL, Coulter), cells were incubated with saturating concentrations of primary antibodies for 30 min at 4° C., washed in FACS buffer, stained with secondary antibodies for 30 min at 4° C. and washed. Dead cells were gated out by forward and side scatter properties. Single or double parameter profiles are shown in log scale (FIG. 8A-C). Monoclonal antibodies used were fluorescein. (FITC)-conjugated RA3-6B2 (B220) (GIBCO), fluorescein (FITC) conjugated KT3 (CD3) (Serotec). PrP was detected with the polyclonal antiserum R340 and a phycoerythrin-conjugated sheep anti-rabbit IgG (Serotec).

### 11.8. Scrapie Infection and Diagnosis

Scrapie infection was carried out as specified in example 10.1. Mice were checked for the development of scrapie symptoms every other day and, once they developed the disease, every day (Büeler, H., Aguzzi, A., Saller, A., Greiner, R. A., Autenried, P., Aguet, M. and Weissmann, C. (1993). Mice devoid of PrP are resistant to scrapie. Cell 73, 1339-1347).

### 11.9. Titration of Infectivity

Prion titers were estimated by determining incubation times to appearance of disease (Prusiner, S. B. (1982). Novel proteinaceous infectious particles cause scrapie. Science 216, 136-144). Tissue homogenates (10%, w/v) in 0.32 M sucrose were prepared as described (Büeler, H., Aguzzi, A., Saller, A., Greiner, R. A., Autenried, P., Aguet, M. and Weissmann, C. (1993). Mice devoid of PrP are resistant to scrapie. Cell 73, 1339-1347). Aliquots of tissue homogenates from two mice sacrificed at the same time after inoculation were pooled and ‘diluted serially in PBS-5% BSA. Swiss CD-1 mice were inoculated i.c. into the right parietal lobe with 30-μl samples using a 26-gauge hypodermic needle. In some cases titration of infectivity was carried out in homozygous Tg20 mice (Fischer, M., Rülicke, T., Raeber, A., Saller, A., Moser, M., Oesch, B., Brandner, S., Aguzzi, A. and Weissmann, C. (1996). Prion protein (PrP) with amino-proximal deletions restoring susceptibility of PrP knockout mice to scrapie. EMBO J. 15, 1255-1264).

Discussion of the Results of Example 11

Prnp^{o/o} mice under the control of a human IRF1-promoter/Em-enhancer expressing high levels of PrP in the spleen underwent prion replication in B-cells and T-cells.

However, Prnp^{o/o} mice expressing PrP (driven by the Lck promoter) at high levels on T lymphocytes but not on B lymphocytes (“T-cell mice”) failed to replicate prions in spleen or thymus and developed no clinical symptoms.

Thus, since overexpression of PrP in the liver of PrnP^{o/o} mice failed to sustain prion replication in liver, spleen or brain (thus showing that PrP^{c} (over)expression alone is not sufficient to allow disease spread), these three experiments markedly point to the B-cells as only species having all the prerequisites required for sustaining prion replication.

Therefore, in conjunction with the findings of examples 1, 2 and 10 (see in particular tables 1 and 2), these results strikingly confirm the crucial role of the B-cells as rate limiting carrier's of prions in that T-cells cannot take up prions on their own but can only acquire them by way of secondary, B-cell-mediated infection.

### Example 12

### Direct Western Blot Analysis of Splenocytes, B Cell, T Cell, and Non-B/Non-T Cell Fractions

Cell fractions were isolated from the spleens of intraperitoneally infected wild-type mice. Cell aliquots (2−5×10⁶ cells) were electrophoresed through SDS polyacrylamide gels either directly (−) or after treatment with 20 mg/ml proteinase K (PK) (+) for 30 min at 37 C. Following electrophoresis, gels were blotted onto nitrocellulose membranes and PrP was visualized with the anti-PrP monoclonal antibody 6H4 and chemiluminescence detection (see FIG. 10). FIG. 10 shows that fractionation of the spleen cells into the carriers of infectivity as identified by the present invention (namely into the B and possibly the T-cells) and into the remaining cells (non B-/T-cells) significantly improves sensitivity of the Western blot.

### Example 13

### B- and T-Cell Depletion of Different Fractions from Human Blood

### Materials and Methods

### 13.1 Separation of blood into its Components

E.g., a scaled-down version of the “three bag” protocol used by the American Red Cross may be used for component separation. Anticoagulated whole blood is centrifuged (SORVALL® SS-34 rotor, Dupont Medical Products Clinical Diagnostics, Wilmington, Del.) at 4300 rpm (2280×g) for 4 minutes at ambient temperature. The supernatant (“crude”) plasma is carefully withdrawn by pipette down to the edge of the buffy coat overlying the red cell sediment, transferred to a new 50 ml tube, and centrifuged at 5800 rpm (4200×g) for 8 minutes at ambient temperature. The supernatant plasma is pipetted into a new tube, leaving behind a very small sedimented pellet. Such pellet is combined with the pellet from the plasma centrifugation step to yield a single white cell and platelet specimen (“human buffy coat fractions”) for purification.

### 13.2. Cohn Fractionation of the Plasma Components

The “crude” plasma fractions obtained at different rotational speeds as above may be pooled.

Approximately 10 ml plasma are then transferred from −70° C. (storage) to −20° C. for overnight “tempering”, then exposed to a final 30-minute thaw inside a 50-ml jacketed reaction beaker connected to a refrigerated circulating bath set at 1° C. to 2° C. The thawed plasma is transferred to a weighed, cold, 15-ml centrifuge tube and centrifuged at 6800 rpm (5600×g) for 15 minutes at 1° C. to 2° C. The pellet is weighed and then frozen at −70° C. (cryoprecipitate).

The supernatant is again placed into the reaction beaker-circulating bath apparatus set at 1° C. to 2° C., and the pH is adjusted to 6.65 to 6.70 with acetate buffer, pH 4.0. (10.9 g sodium acetate, 24 g glacial acetic acid, 71 ml water). Slowly, over a period of 1 hour, repeated small amounts of cold 95-percent ethanol are added to achieve a final ethanol concentration of 20 percent. After addition of one half of the ethanol, the pH is verified to be in the range of 6.80 to 7.00, and circulating bath temperature is lowered from 1° C. to 2° C. to-5° C. The plasma-ethanol mixture is transferred to a weighed, cold centrifuge tube and centrifuged at 6800 rpm (5600×g) for 15 minutes at −5° C. The pellet is weighed and frozen at −70° C. (fraction I+II+III).

The supernatant is again placed into the reaction beaker-circulating bath apparatus set at −5° C. The pH is adjusted to 5.16 to 5.22 with acetate buffer in 20-percent ethanol, pH 4.0, and then further adjusted to a final pH of 5.75 with 1M NaHCO₃. Slowly, over a period of 1 hour, small quantities of cold 95-percent ethanol are added to achieve a final ethanol concentration of 40 percent and a final pH of 5.92 to 5.98. The plasma-ethanol mixture is transferred to a weighed, cold centrifuge tube and centrifuged at 6800 rpm (5600×g) for 15 minutes at −5° C. The pellet is weighed and frozen at −70° C. (fraction IV₁/IV₄).

The supernatant is placed into a tube containing 2 mg of filter aid per ml of supernatant, mixed, and filtered through a 20-ml syringe containing a filter (CPX70, Cuno, Meriden, Conn.). The filtrate is placed into the reaction beaker-circulating bath apparatus set at −5° C. The pH is adjusted to 4.78 to 4.82 by slowly adding acetate buffer in 40-percent ethanol, pH 4.0. The plasma mixture is placed into a weighed, cold centrifuge tube and centrifuged at 6800 rpm for 15 minutes at −5° C. The pellet is weighed and frozen at −70° C. (fraction V). The supernatant is also frozen at −70° C. (fraction V supernatant).

### 13.3. B- and T-Cell Depletion of Human Buffy Coat Fractions

Human buffy coat fractions are depleted of B and T-lymphocytes by using anti-CD19 (B-cells) or anti-CD3 (T-cells) antibodies coupled to a solid support. Antibodies will be linked covalently to a solid support consisting of a plastic or metal filter or membrane devices. Covalent attachment methods are well known to those skilled in the art. Following attachment of antibodies to the solid support, unspecific binding sites will be blocked with serum, proteins or other blocking agents to minimize non-specific binding. Buffy coat fractions are then passed over this support to deplete B or T lymphocytes.

### 13.4. B- and T-Cell Depletion of Plasma Fractions

Since the high speed centrifugation steps of the “crude” plasma fractions may lead to the formation of cellular debris, B and T-cell depletion as described above, is carried out preferably before Cohn fractionation. However, still more preferably, B- and T-cell depletion is already carried out with the first “crude” plasma fraction obtained at 2280×g. That is to say, preferably already the first “crude” plasma fraction (2280×g), but also the second “crude” plasma fraction (4200×g) (or, in the alternative, the pooled fraction arising therefrom) are plasma precursors suitable for the B and T-cell depletion step(s) as contemplated by the invention.

### 13.5. B- and T-Cell Depletion of the Cryoprecipitate Fraction

Optionally, also so the cryoprecipitate fraction may be treated as described above for the human buffy coat fractions.

### Example 14

### Therapeutic B- and T-Cell Depletion by Combination Therapy with Cyclophosphamide & Dexamethasone

### Materials & methods

### 14.1. Mice & Inoculation

For infection studies 8-10 week old C57/B16 mice were inoculated intraperitoneally with 100 ml of different dilutions of a 1% brain homogenate from mice infected with the Rocky Mountain Laboratory scrapie strain.

### 14.2. B and T-Cell Depletion

To deplete B- and T-cell populations and prevent recovery, mice were initially treated intraperitoneally with an dose of 250 mg/kg Cyclophosphamide and 10 mg/kg Dexamethasone.

Depletion was repeated every 5-6 days. 2^{nd} and 3^{rd} injections were performed with 200 mg/kg Cyclophosphamide and 10 mg/kg Dexamethasone. Starting from injection No. 4, animals were treated weekly with 160 mg/kg Cyclophosphamide and 10 mg/kg Dexamethasone for 10 more weeks. Depletion of B- and T-cell population was monitored by FACS-analysis prior to first injection and inoculation and after animals were sacrificed. Depletion of immunoglobulins was detected by determination of IgG and IgM levels in serum of experimental animals by ELISA-assay.

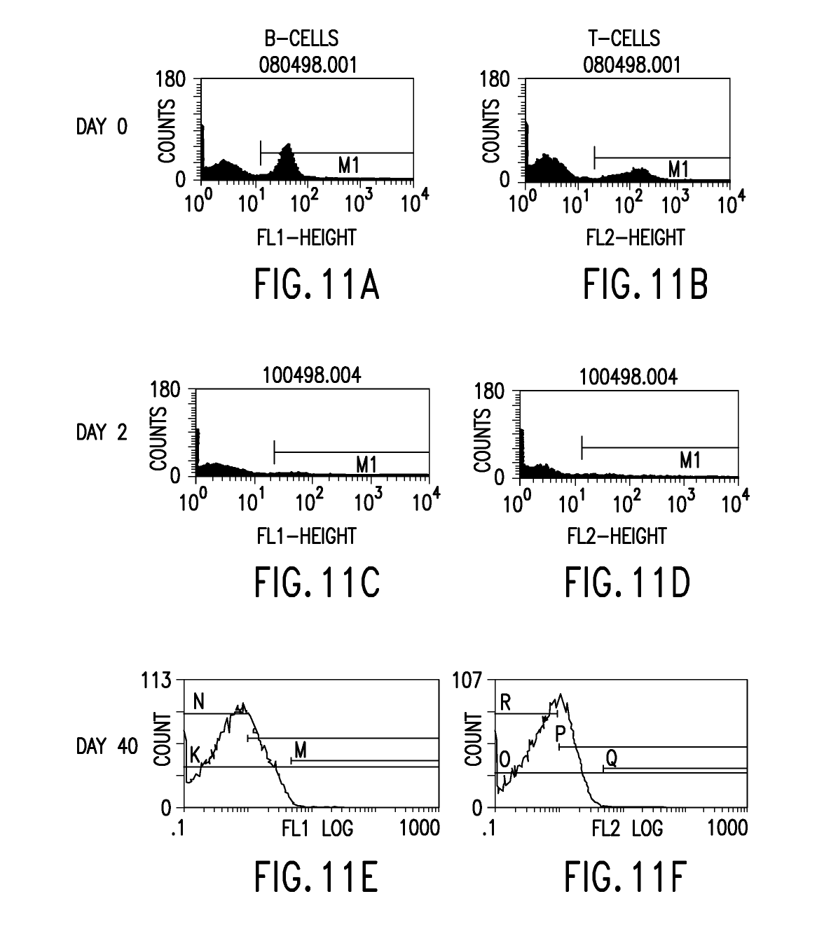

### 14.3. FACS-Analysis (FIG. 11)

Thirty ml of peripheral blood were pelleted, washed and incubated with fluorescence-labeled antibodies recognizing B or T-cell marker proteins (CD19 and CD3) after erythrocyte lysis and fixation. Probes were analyzed with a Becton-Dickinson FACScan instrument. All data acquisition and analysis was performed with CellQuest software (Becton-Dickinson).

### 14.4. Western Blot Analysis

Spleen homogenates were prepared and digested with 20 mg/ml Proteinase K for 30 min at 37° C. One-hundred-twenty of protein were then separated on a 12% SDS-PAGE, transferred onto nitrocellulose membranes, probed with monoclonal antibody 1B3 and developed by enhanced chemoluminescence (ECL).

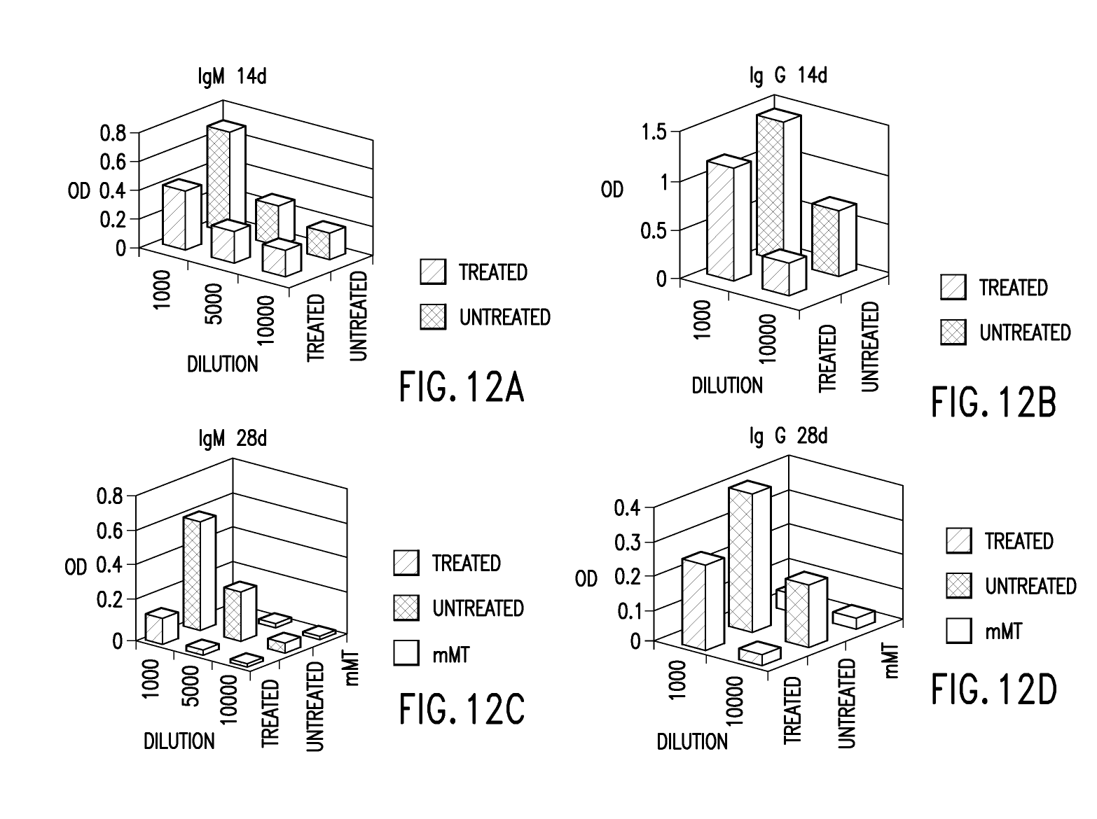

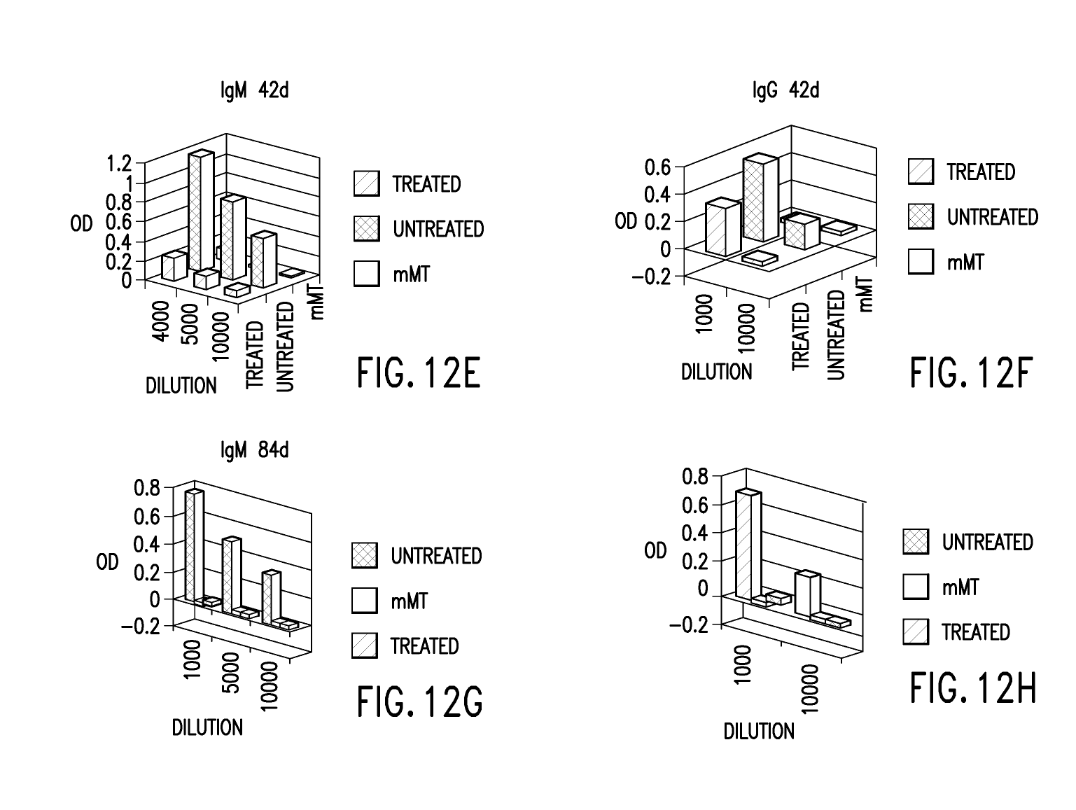

14.5. ELISA (FIG. 12)

Plates were coated overnight with unlabeled anti IgG or IgM antibodies diluted 1:1000 in 50 mM NaH₂PO₄. After blocking unspecific binding with 3% BSA in PBS containing 0.1% Tween 20 plates were incubated with serum of experimental animals at different dilutions (1:100, 1:500, 1:1000, 1:5000). After washing, serum-IgGs and IgMs were detected with HRP-conjugated antibody detecting IgA, IgE, IgG and IgM. Plates were developed for 50 min. with ABTS (5 mg 2,2′-azino-di-aethyl-benzthiazolinsulfonate in 0.1 M NaH₂PO₄, +1, 8 ml H₂O₂/ml).

### 14.6. Infectivity Bioassays

Spleen homogenates (10% in 0.32M sucrose) were prepared from infected mice after 42 days, and 30 ml (diluted 1:10 in PBS containing 1% BSA) were administered intracerebrally into groups of 4 tga20 mice for each sample. The incubation time until development of terminal scrapie sickness was determined.

### Results

C57/B16 mice were treated with dexamethasone and cyclophosphamide (Table 11). Experimental groups were inoculated with different dilutions (10⁻¹-10⁻⁴) of RML4.1 as described. Animals were treated with Dexamethasone and Cyclophosphamide as described above in “materials and methods” at different time points. In Groups I-IV treatment was started 2 days prior to inoculation with RML. Groups V-VIII were first injected with Dexamethasone and Cyclophosphamide at the day of prion administration while groups IX and X were first treated with lymphocyte depleting drugs 10 days after inoculation (for groups IX and X, see also FIG. 14). Control groups XI and XII were not treated.

TABLE 11
Animals were treated with Dexamethasone and Cyclophosphamide
as described in Material & Methods at different time points.
In Groups I-IV treatment was started 2 days prior to inoculation
with RML. Groups V-VIII were first injected with Dexamethasone
and Cyclophosphamide at the day of prion administration while
groups IX and X were first treated with lymphocyte depleting
drugs 10 days after inoculation. Control groups XI and XII
were not treated.

| | | | | |
|---|---|---|---|---|
| | | | 1st | |
| Group | inoculation | Depletion | depletion | Analysis |

| | | | | | |
|---|---|---|---|---|---|
| I | RML 10⁻¹ | + | −2 | d | 4 |
| II | RML 10⁻² | + | −2 | d | 4 |
| III | RML 10⁻³ | + | −2 | d | 4 |
| IV | RML 10⁻⁴ | + | −2 | d | 4 |
| V | RML 10⁻¹ | + | 0 | d | 4 |
| VI | RML 10⁻² | + | 0 | d | 4 |
| VII | RML 10⁻³ | + | 0 | d | 4 |
| VIII | RML 10⁻⁴ | + | 0 | d | 4 |
| IX | RML 10⁻¹ | + | +10 | d | 2 |
| X | RML 10⁻⁴ | + | +10 | d | 2 |

| | | | | |
|---|---|---|---|---|
| XI | RML 10⁻¹ | − | —^{a} | 2 |
| XII | RML 10⁻⁴ | − | — | 2 |

Prior to drug treatment PBL samples of all animals were tested by FACS-analysis (FIG. 11, day 0) to show presence and detectability of B and T-Lymphocytes. Depletion was monitored in groups I-IV 2 days after first injection of Dexamethasone & Cyclophosphamide directly before inoculation (FIG. 11, day 2). A further analysis was performed 40 days after inoculation (FIG. 11, day 40). FACS-analysis shows efficient depletion of B- and T-cell population in PBLs. Since FACS-analysis does only apply to cells floating in the bloodstream, the inventors decided to determine IgM and IgG levels in the serum to monitor overall presence of immunoglobulin-secreting cells. ELISA assays of serum-probes from animals taken every 2 weeks showed a slow decrease of IgG and IgM levels (FIG. 12).

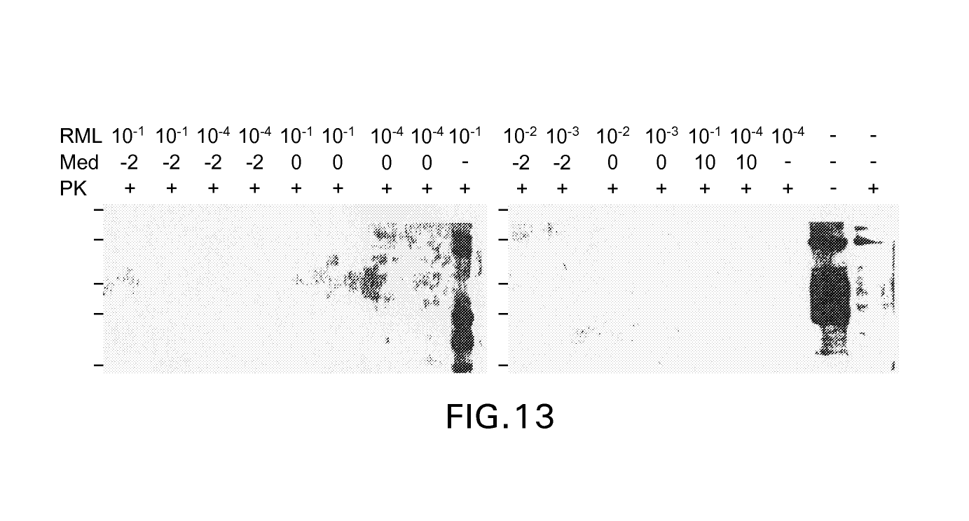

42 days after inoculation with RML prions 2-4 mice of each experimental group were killed to assay spleens for accumulation of PrP^{Sc} and infectivity using western blots and the infectivity bio-assay. Western blot analysis of the small and atropic spleens of infected animals showed no detectable accumulation of proteinase K resistant PrP^{Sc} (FIG. 13), while accumulation is easily detectable at such a late stage in unaffected, spleens of infected animals not treated with: B- and T-cell depleting drugs (FIG. 13). To monitor for accumulation of infectivity in spleens of infected animals with and without drug, treatment tga20 indicator mice were also inoculated intracerebrally with spleen homogenates of experimental animals.

Indicator animals inoculated with spleen homogenates from untreated mice succumbed at 85, and 87d after inoculation, respectively.

Tga20 mice inoculated with spleen homogenates from depleted animals stayed healthy until 153d (13/12/98) after transmission. Up to this time point this reflects at least a significant decrease if not a complete absence in the amount of infectivity accumulated in the spleens of B and T-cell depleted animals. Therefore, the therapeutic results obtained, especially if considered together with the remaining disclosure of the present application, are a further piece of evidence pointing to the involvement of a B-cell mediated spread mechanism with secondary T-cell infection.

### Example 15

### Effect of B-Cell Depletion of Mice with Ciamexone and/or Imexon on Susceptibility to Peripherally Administered Prions

For infection studies 8-10 week old C57/B16 mice are inoculated intraperitoneally with 100 ml of different dilutions of a 1% brain homogenate from mice infected with the Rocky Mountain Laboratory scrapie strain. To deplete B-cell population and prevent recovery, groups of at least 4 mice are exposed to various amounts of ciamexone (1-100 mg/kg) or imexon (50-150 mg/kg) delivered either intraperitoneally or in the drinking water. Depletion of B-cell population is monitored by FACS-analysis of PBLs. Depletion of immunoglobulins can be detected by determination of IgG and IgM levels in serum of experimental animals by ELISA-assays.

To determine accumulation of PrP^{Sc} in the spleens of experimental animals, western blots are performed. Spleen homogenates of infected animals are prepared 34 days after inoculation and digested with 20 mg/ml Proteinase K for 30 min at 37° C. to detect protease resistant protein. Furthermore, infectivity bioassays are performed to detect small amounts of infectious agent accumulated in spleens and brains of experimental animals. Spleen, splenic fractions (B-cells, if detectable, T-cells and non B-/T-fraction) and brain homogenates (10% in 0.32M sucrose) are prepared from infected mice and 30 ml (diluted 1:10 in PBS containing 1% BSA) are administered intracerebrally into groups of 4 tga20 mice per sample. The incubation time until development of terminal scrapie sickness is determined and infectivity titers are calculated.

Since FACS-analysis does only apply to cells floating in the bloodstream, IgM and IgG levels in the serum have to be determined to monitor overall presence of immunoglobulin-secreting cells. ELISA assays of serum-probes from animals should be taken every 2 weeks to verify the decrease of IgG and IgM levels.

### Example 16

### Effect of B-Cell Depletion of Mice with Rituxin in Susceptibility to Peripherally Administered Prions

For the administration, of rituxin, an experimental protocol as the one set out in example 15 is followed. The doses of administration are varied according to the manufacturer's instructions.

### Example 17

### Effect of T-Cell Depletion of Mice with Cyclosporin A on Susceptibility to Peripherally Administered Prions

For the administration of cyclosporin A, an experimental protocol as the one set out in example 15 is followed. The doses of administration are varied according to the manufacturer's instructions.

### Example 18

### Effect of Immunodepletion of Mice with Combined or Sequential B- and/or T-Cell Depletants on Susceptibility to Peripherally Administered Prions

For the combined or sequential administration of various B and/or T-cell depletants as the ones of the foregoing examples or of any others as contemplated by the present invention, an experimental protocol as the one set out in examples 14-17 is followed, according to the specific combination or sequence chosen. The doses of administration are varied according to the manufacturer's instructions together with the indications on mutual compatibility.

Still other variations and modifications of the specific embodiments or the pure illustrative-examples of the invention as set forth herein will be readily apparent to those skilled in the art. Accordingly, the invention is intended to be limited solely in accordance with the appended claims.

### FURTHER REFERENCES CITED IN THE PRESENT APPLICATION

1. Hill, A. F. et al. The same priori strain causes vCJD and BSE. Nature 389, 448-450 (1997).
2. Bruce, M. E. et al. Transmissions to mice indicate that ‘new variant’ CID is caused by the BSE agent. Nature 389, 498-501 (1997).
3. Kitamoto, T., Muramoto, T., Mohri, S., Dohura, K. & Tateishi, J. Abnormal isoform of prion protein accumulates in follicular dendritic dells in mice with Creutzfeldt-Jakob disease. J. Virol. 65, 6292-6295 (1991).
4. Lasmezas, C. I. et al. Immune system-dependent and -independent replicaiton of the scrapie agent: J. Virol. 70, 1292-1295 (1996).
5. Shinkai, Y. et al. RAG-2-deficient mice lack mature lymphocytes owing to inability to initiate V(D)J rearrangement. Cell 68, 855-867 (1992).
6. Mombaerts, P. et al. RAG-1-deficient mice have no mature B and T lymphocytes. Cell 68, 869-877 (1992).
7. Huang, S. et al. Immune response in mice that lack the interferon-γ receptor. Science 259, 1742-1745 (1993).
8. Müller, U. et al. Functional role of type I and type II interferons in antiviral defense. Science 264, 1918-1921 (1994).
9. Rahemtulla, A. et al. Normal development and function of CD8⁺ cells but markedly decreased helpercell activity in mice lacking CD4. Nature 353, 180-184 (1991).
10. Fung Leung, W. P. et al. CD8 is needed for development of cytotoxic T cells but not helper T cells. Cell 65, 143-449 (1991).
11. Ziilstra, M. et al. B 2-Microgiobuin-deficient mice lack CD4⁺8⁺ cytolytic T cells. Nature 344, 742-746 (1990).
12. Kagi, D. et al. Cytotoxicity mediated by T cells and natural killer cells is greatly impaired in perforindeficient mice, Nature 369, 31-37 (1994).
13. Kitamura, D., Roes, J., Kuhn, R. & Rajewsky, K. A B-cell-deficient mouse by targeted disruption of the membrane exon of the immunoglobulin Mu-chain gene. Nature 3 50, 423-426 (1991).
14. Fischer, M. et al. Prion protein.(PrP) with amino-proximal deletions rstoring susceptibility of PrP-knockout mice to scrapie. EMBO J. 15, 1255-1264 (1996).
15. Büeler, H. R. et al. Normal development and behaviour of mice lacking the neuronal cell-surface PrP protein. Nature 356, 577-582 (1992).
16. Büeler, H. R. et al. Mice devoid of PrP are resistant to scrapie. Cell 73, 1339-1347 (1993).
17. Fraser, H. et al. Replication of scrapie in spleens of scid mice follows reconstitution with wild-type mouse bone marrow. J. Gen. Virol. 11, 1935-1940 (1996).
18. Nonoyama, S., Smith, F. O., Bernstein, I. D. & Ochs, H. D. Strain-dependent leakiness of mice with severe combined immune deficiency. J. Immunol. 150, 3817-3824 (1993).
19. Bosma, M. J. & Carroll, A. M. The SCID mouse mutant: definition, characterization, and potential uses. Annu. Rev. Immunol. 9, 323-350 (1991).
20. Eigen, M. Prionics or the kinetic basis of prion diseases. Biophys. Chem. 63, A1-18 (1996).
21. Hill, A. F., Zeidler, M., Ironside, J. & Collinge, J. Diagnosis of new variant Creutzfeldt-Jakob disease by tonsil biopsy. Lancet 349, 99 (1997).
22. Rothe, J. et al. Mice lacking the tumour-necrosis factor receptor 1 are resistant to TNF-mediated toxicity but highly susceptible to infection by Listeria monocytogenes. Nature 364, 798-802 (1993).
23. Le Hir, M. et al. Differentiation of follicular dendritic cells and full antibody responses require tumor necrosis factor receptor-1 signaling. J. Exp. Med. 183, 2367-2372 (1996).
24. Humphrey, J. H., Grennan, D. & Sundaram; V. The origin of follicular dendritic cells in the mouse and the mechanism of trapping of immune complexes on them. Eur. J. Immunol. 14, 859-864 (1984).
25. Blattler, T. et al. Transfer of scrapie infectivity from spleen to brain depends on interposed PrP-expressing tissue. Nature 389, 69-73 (1997).
26. Farquhar, C. F., Somerville, R. a. & Ritchie, L. A. Postmortem immunodiagnosis of scrapie and bovine spongiform encephalopathy. J. Virol. Meth. 24, 215-221 (1989).
27. Kalinke, U. et al. The role of somatic mutation in the generation of the protective humoral immune response against vesicular stomatitis virus. Immunity 5, 639-652 (1996).
28. Prusiner, S. B. et al. Measurement of the scrapie agent using an incubation time interval assay. Neurol. 11, 353-358 (1982).
29. Brandner, S. et al. Normal host prion protein (PrPc) required for scrapie spread within the central nervous system. Proc. Natl Acad. Sci. USA 93, 13148-13151 (1996).

Above listed references as well as further references (articles or Patents) cited in the specification as above are hereby included by reference to the disclosure of the present application.

1. Use of depletants selected from the group containing B-cell depletants, T-cell depletants and B and T-cell depletants for the manufacture of a medicament for the treatment or prevention of transmissible spongiform encephalopathy in infected humans or animals.

2. Use according to claim 1, characterized in that said B-cell depletants comprise anti B-cell antibodies.

3. Use according to claim 2, characterized in that said anti B-cell antibodies comprise anti-umM antibodies.

4. Use according to claim 2, characterized in that said anti B-cell antibodies comprise LR1 antibodies.

5. Use according to claim 2, characterized in that said anti B-cell antibodies comprise B220 antibodies.

6. Use according to claim 2, characterized in that said anti B-cell antibodies comprise rituximab.

7. Use according to claim 1, characterized in that said anti B-cell depletants comprise chemical compounds.

8. Use according to claim 7, characterized in that said chemical compounds comprise imexon.

9. Use according to claim 7, characterized in that said chemical compounds comprise ciamexone.

10. Use according to claim 1, characterized in that said T-cell depletants comprise anti T-cell antibodies.

11. Use according to claim 10, characterized in that said anti T-cell antibodies comprise Thy1.2 antibodies.

12. Use according to claim 11, characterized in that said T-cell depletants comprise chemical compounds.

13. Use according to claim 12, characterized in that said T-cell depletants comprise cyclosporin A.

14. Use according to claim 1, characterized in that said B- and T-cell depletants comprise a combination of cyclophosphamide and dexamethasone either in a combined dosage form or in separate dosage forms.

15. A product comprising cyclophosphamide and dexamethasone as a combined preparation for the simultaneous, separate or sequential use in the treatment or prevention of transmissible spongiform encephalopathy in infected humans or animals.

16. Use of body fluid or tissue derived cell or cell debris containing products for the prevention of transmissible encephalopathy spread in human or animal populations characterized in that said body fluid or tissue derived products are selected from the group containing B-cell depleted, T-cell depleted and B and T-cell depleted body fluid or tissue derived products.

17. Buffy coat, characterized in that it has been depleted in vitro of the cells selected from the group containing B-cells, T-cells and B and T-cells.

18. Method for the provision of a buffy coat as claimed in claim 17 characterized in that said buffy coat is contacted with antibodies selected from the group containing anti B-cell, anti T-cell and anti B and T-cell antibodies that are linked to a solid support.

19. Method for the purification of plasma characterized in that such plasma or a precursor used in the preparation thereof is contacted with antibodies selected from the group containing anti B-cell, anti T-cell and anti B and T-cell antibodies that are linked to a solid support.

20. Method for the manufacture of plasma or buffy coat, characterized in that said plasma or buffy coat are isolated from B-cell deficient animals.

21. Method according to claim 20, characterized in that said B-cell deficient animals are produced by removing or inhibiting expression of B-cell” related genes contained therein.

22. Assay method for the determination of the presence of tse-infected cells selected from the group containing B-cells, T-cells and B and T-cells in humans or animals or in body fluid or tissue derived products isolated therefrom, characterized in that said method comprises the steps of: extracting the cells selected from the group comprising B-cells, T-cells and B and T-cells from body fluids or from tissue or from products derived therefrom and inoculating said cells into the cerebrum of a test animal, development of transmissible spongiform encephalopathy in said test animal indicating presence of said tse-infected cells.

23. Assay method for the ‘determination of the presence of tse-infected cells selected from the group containing B-cells, T-cells and B and T-cells in humans or animals or in body fluid or tissue derived products isolated therefrom, characterized in that the cells are subjected to a Western blot analysis with anti-PrP antibody either directly and after having been digested with proteinase K.

24. Assay method for the monitoring of the progress of transmissible spongiform encephalopathy or of the therapy against such disease in humans or animals characterized in that is comprises the steps of claims 22 or 23.

25. An antibody directed against tse-infected cells selected from the group containing B-cells and T-cells, characterized in that said antibody shows specificity to a tse-infected marker of each of the cells selected from the group containing B-cells and T-cells and is obtainable by immunization of host animals with tse-cells infected cells each selected from the group containing B-cells and T-cells.

26. Use of the antibody according to claim 25 in a diagnostic assay.

27. A medicament comprising the antibody of claim 25.

28. A ligand capable of identification of tse-infected cells selected from the group containing B-cells and T-cells, characterized in that specific interaction between said ligand said tse-infected-cell is based on the infectivity of said cell.

29. Use of a ligand according to claim 28 in a method of analysis of said cells.

30. Use according to claim 29 characterized in that said cells are intact.

31. Use according to claim 30 in histochemical analysis of the whole cells selected from the group containing B-cells and T-cells mounted on microscope slides.


SEQUENCE LISTING (5 sequences)

SEQ ID NO: 1 - DNA, 24 bp, Mus musculus
ggatccgcagaccgattctggacg

SEQ ID NO: 2 - DNA, 26 bp, Artificial sequence
  5′ terminal primer
tgtcggatccagcagaccgattctgg

SEQ ID NO: 3 - DNA, 23 bp, Artificial sequence
  3′ terminal primer
tccccagcatgtagccaccaagg

SEQ ID NO: 4 - DNA, 21 bp, Artificial sequence
  5′ terminal primer
tttctagaggagccaggctgc

SEQ ID NO: 5 - DNA, 23 bp, Artificial sequence
  3′ terminal primer
agggatcctcgactaaggagtgg